(19) United States
(12) Patent Application Publication
(10) Pub. No.: US 2025/0297184 A1
(43) Pub. Date: Sep. 25, 2025

(54) FLUORINE-CONTAINING ETHER COMPOUND, LUBRICANT FOR MAGNETIC RECORDING MEDIA, AND MAGNETIC RECORDING MEDIUM

(71) Applicant: Resonac Corporation, Tokyo (JP)

(72) Inventors: Yutaka TANJI, Tokyo (JP); Takuya UNO, Tokyo (JP)

(73) Assignee: Resonac Corporation, Tokyo (JP)

(21) Appl. No.: 18/860,799

(22) PCT Filed: Sep. 29, 2023
(86) PCT No.: PCT/JP2023/035678

§ 371 Date: Oct. 28, 2024

(30) Foreign Application Priority Data
Sep. 30, 2022 (JP) 2022-158414

(51) Int. Cl. C10M 107/38; C08G 65/331; C10N 40/18; G11B 5/725

(52) U.S. Cl. CPC C10M 107/38; C08G 65/3312; G11B 5/7257; C10M 2213/0606; C10N 2040/18

(57) ABSTRACT

A fluorine-containing ether compound represented by the following formula.

R¹—CH₂—R²[—CH₂—R³—CH₂—R²]ₓ—CH₂—R⁴

(x is an integer of 1 or 2, R² is a perfluoropolyether chain, R³ is represented by Formula (2), and R¹ and R⁴ are each a terminal group having 1 to 50 carbon atoms, which has 1 to 4 polar groups)

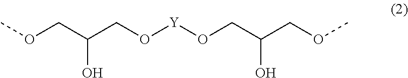

### TECHNICAL FIELD

The present invention relates to a fluorine-containing ether compound, a lubricant for a magnetic recording medium, and a magnetic recording medium. Priority is claimed on Japanese Patent Application No. 2022-158414, filed on Sep. 30, 2022, the content of which is incorporated herein by reference.

### BACKGROUND ART

Development of a magnetic recording medium suitable for a high recording density has been advanced in order to increase a recording density in a magnetic recording and reproducing device.

In the related art, as the magnetic recording medium, there is a magnetic recording medium in which a recording layer is formed on a substrate and a protective layer made of carbon and the like is formed on the recording layer. The protective layer protects the information recorded in the recording layer and also improves the slidability of a magnetic head. However, the durability of the magnetic recording medium is not sufficiently obtained simply by providing the protective layer on the recording layer. Therefore, a lubricant is generally applied to a surface of the protective layer to form a lubricating layer.

As a lubricant used in a case of forming a lubricating layer of a magnetic recording medium, for example, a lubricant that contains a compound having a polar group such as a hydroxy group and an amino group at a terminal of a fluorine-based polymer having a repeating structure including —CF₂— has been proposed.

For example, Patent Document 1 and Patent Document 2 disclose a fluorine-containing ether compound having a skeleton where two perfluoropolyether chains are bonded through divalent linking groups having methylene groups (—CH₂—) bonded to both ends of a glycerin structure (—O—CH₂—CH(OH)—CH₂—O—), in which terminal groups that are organic groups having a polar group are bonded to both terminals of the skeleton through the methylene groups.

Patent Document 3 and Patent Document 4 disclose a fluorine-containing ether compound having a skeleton where two perfluoropolyether chains are bonded through divalent linking groups including a methylene group (—CH₂—) and a group (—CH(OH)—) obtained by substituting one of hydrogen atoms of a methylene group with a hydroxy group, and having two hydroxy groups, in which terminal groups that are organic groups having a polar group are bonded to both terminals of the skeleton through the methylene groups.

Patent Document 5 discloses a fluorine-containing ether compound having a skeleton where two or three perfluoropolyether chains are bonded to each other through divalent linking groups including a methylene group (—CH₂—) and a group (—CH(OH)—) obtained by substituting one of hydrogen atoms of a methylene group with a hydroxy group, and having two hydroxy groups, in which terminal groups that are organic groups having a polar group are bonded to both terminals of the skeleton through the methylene groups.

Patent Document 6 and Patent Document 7 disclose a fluorine-containing ether compound having a skeleton where two perfluoropolyether chains are bonded to each other through divalent linking groups including a benzene ring or an alicyclic structure, in which terminal groups that are organic groups having a polar group and are bonded to both terminals of the skeleton through methylene groups (—CH₂—).

### CITATION LIST

### Patent Documents

Patent Document 1: U.S. patent Ser. No. 10/540,997
Patent Document 2: PCT International Publication No. WO2021/251335
Patent Document 3: PCT International Publication No. WO2021/020066
Patent Document 4: Japanese Patent No. 6804981
Patent Document 5: U.S. patent Ser. No. 10/262,685
Patent Document 6: PCT International Publication No. WO2021/065380
Patent Document 7: PCT International Publication No. WO2022/113854

### SUMMARY OF INVENTION

### Technical Problem

In recent years, it is required to further reduce a magnetic spacing (a distance between a magnetic head and a magnetic layer of a magnetic recording medium) in order to increase the capacity of the magnetic recording medium. Therefore, it is required to further reduce a thickness of a lubricating layer in the magnetic recording medium.

However, in general, in a case where the thickness of the lubricating layer is reduced, the chemical substance resistance of the magnetic recording medium tends to be decreased. In addition, in a case where the thickness of the lubricating layer is reduced, a sufficient film thickness cannot be maintained in order to satisfy the function of the lubricating layer in a case of occurrence of spin-off. The spin-off is a phenomenon in which the lubricant is scattered or evaporated due to centrifugal force and heat generated by the rotation of the magnetic recording medium.

The present invention has been made in view of the circumstances, and an object of the present invention is to provide a fluorine-containing ether compound, which can form a lubricating layer having excellent chemical substance resistance and having spin-off suppressed, and can thus be suitably used as a material for a lubricant for a magnetic recording medium.

Another object of the present invention is to provide a lubricant for a magnetic recording medium capable of forming a lubricating layer which includes the fluorine-containing ether compound of the present invention and has good chemical substance resistance and a high spin-off suppressing effect.

Still another object of the present invention is to provide a magnetic recording medium having a lubricating layer which includes the fluorine-containing ether compound of the present invention and has good chemical substance resistance and a high spin-off suppressing effect.

### Solution to Problem

The present invention includes the following aspects.

A first aspect of the present invention provides the following fluorine-containing ether compound.
[1]A fluorine-containing ether compound represented by Formula (1).

R¹—CH₂—R²[—CH₂—R³—CH₂—R²]ₓ—CH₂—R⁴ (1)
(in Formula (1), x represents an integer of 1 or 2; R² is a perfluoropolyether chain; (x+1) pieces of R²'s may be partially or entirely the same as or different from each other; R³ is a divalent linking group represented by Formula (2); in a case where x is 2, two R³'s may be the same as or different from each other; R¹ and R⁴ are each a terminal group having 1 to 50 carbon atoms, which has 1 to 4 polar groups; and R¹ and R⁴ may be the same as or different from each other)

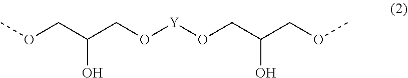

(in Formula (2), Y represents an acyclic divalent saturated hydrocarbon group having 2 to 8 carbon atoms, which may have an ether oxygen atom between carbon atoms)

It is preferable that the fluorine-containing ether compound of the first aspect of the present invention has the following features described in [2] to [10]. The features described in [2] to [10] are also preferably any of combinations of two or more thereof.
[2] The fluorine-containing ether compound according to [1],
in which Formula (2) is a linking group represented by Formula (2-1) or (2-2).

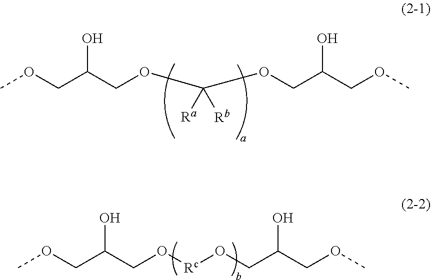

(in Formula (2-1), a represents an integer of 2 to 8; a pieces of R^{a}'s and R^{b}'s each independently represent a hydrogen atom or a methyl group; a total number of carbon atoms included in a pieces of (—CR^{a}R^{b}—)'s is 2 to 8; an oxygen atom at a left terminal of Formula (2-1) is bonded to a methylene group on an R¹ side in Formula (1), and an oxygen atom at a right terminal is bonded to a methylene group on an R⁴ side in Formula (1))
(in Formula (2-2), b represents an integer of 2 to 4; b pieces of R^{c}s each independently represent —CH₂CH₂—, —CH₂CH₂CH₂—, —CH(CH₃)CH₂—, or —CH₂CH(CH₃)—; a total number of carbon atoms included in b pieces of R's is 4 to 8; an oxygen atom at a left terminal of Formula (2-2) is bonded to the methylene group on the R¹ side in Formula (1), and an oxygen atom at a right terminal is bonded to the methylene group on the R⁴ side in Formula (1)).
[3] The fluorine-containing ether compound according to [1] or [2],
in which R¹ and R⁴ in Formula (1) are each independently a terminal group represented by Formula (3).

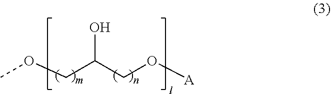

(in Formula (3), 1 represents an integer of 1 to 3; 1 pieces of m's each independently represent an integer of 1 to 6; 1 pieces of n's each independently represent an integer of 1 to 6; at least one of m and n is 1 in one repeating unit; and A represents an alkyl group which may have a polar group, an organic group including a carbon-carbon unsaturated bond, which may have a polar group, or a hydrogen atom)
[4] The fluorine-containing ether compound according to any one of [1] to [3],
in which R¹ and R⁴ in Formula (1) are each independently a terminal group represented by Formula (3-1) or (3-2).

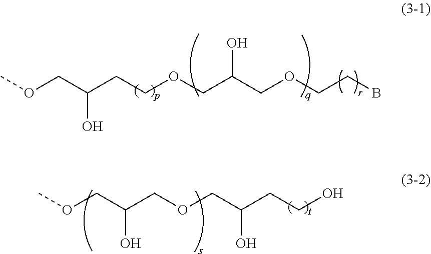

(in Formula (3-1), p represents an integer of 0 to 3, q represents an integer of 0 to 2, and r represents an integer of 1 to 5; a total value of p and r is 1 to 5; and B represents a polar group)
(in Formula (3-2), s represents an integer of 0 to 2, and t represents an integer of 1 to 5).
[5] The fluorine-containing ether compound according to any one of [1] to [4],
in which R¹ and R⁴ in Formula (1) are the same as each other.
[6] The fluorine-containing ether compound according to any one of [1] to [5],
in which a total number of the polar groups contained in R¹ and the polar groups contained in R⁴ in Formula (1) is 2 to 6.
[7] The fluorine-containing ether compound according to any one of [1] to [6],
in which all of the polar groups contained in R¹ and the polar groups contained in R⁴ in Formula (1) are hydroxy groups.
[8] The fluorine-containing ether compound according to any one of [1] to [7],
in which the (x+1) pieces of R²'s in Formula (1) are each independently a perfluoropolyether chain represented by Formula (4).

—(CF₂)_{w1}—O—(CF₂O)_{w2}—(CF₂CF₂O)_{w3}—(CF₂CF₂CF₂O)_{w4}—(CF₂CF₂CF₂CF₂O)_{w5}—(CF₂)_{w6}— (4)
(in Formula (4), w2, w3, w4, and w5 each indicate an average degree of polymerization and each independently represent 0 to 20, provided that all of w2, w3, w4, and w5 are not 0 at the same time; w1 and w6 each indicate an average value of the number of CF₂'s and each independently represent 1 to 3; and (CF₂O), (CF₂CF₂O), (CF₂CF₂CF₂O), and (CF₂CF₂CF₂CF₂O), which are repeating units in Formula (4) are not particularly limited in the sequence order)
[9] The fluorine-containing ether compound according to any one of [1] to [7],
in which the (x+1) pieces of R²'s in Formula (1) are each independently any one selected from perfluoropolyether chains represented by Formulae (4-1) to (4-4).

—CF₂—(OCF₂CF₂)ₕ—(OCF₂)ᵢ—OCF₂— (4-1)
(in Formula (4-1), h and i each indicate an average degree of polymerization, h represents 1 to 20, and i represents 0 to 20)

—CF₂CF₂—(OCF₂CF₂CF₂)ⱼ—OCF₂CF₂— (4-2)
(in Formula (4-2), j indicates an average degree of polymerization and represents 1 to 15)

—CF₂CF₂CF₂—(OCF₂CF₂CF₂CF₂)ₗ—OCF₂CF₂CF₂— (4-3)
(in Formula (4-3), k indicates an average degree of polymerization and represents 1 to 10)

—(CF₂)_{w7}—O—(CF₂CF₂CF₂O)_{w8}—(CF₂CF₂O)_{w9}—(CF₂)_{w10}— (4-4)
(in Formula (4-4), w8 and w9 each indicate an average degree of polymerization and each independently represent 1 to 20; and w7 and w10 are each an average value indicating the number of CF₂'s and each independently represent 1 or 2)
[10] The fluorine-containing ether compound according to any one of [1] to [9],
in which the fluorine-containing ether compound has a number-average molecular weight in a range of 500 to 10,000.

A second aspect of the present invention provides the following lubricant for a magnetic recording medium.
[11]A lubricant for a magnetic recording medium, including:
   the fluorine-containing ether compound according to any one of [1] to [10].

A third aspect of the present invention provides the following magnetic recording medium.
[12]A magnetic recording medium including, in the following order, on a substrate, at least:
   a magnetic layer;
   a protective layer; and
   a lubricating layer,
   in which the lubricating layer includes the fluorine-containing ether compound according to any one of [1] to [10].

It is preferable that the magnetic recording medium according to the third aspect of the present invention has the following features described in [13].
[13] The magnetic recording medium according to [12],
   in which an average film thickness of the lubricating layer is 0.5 nm to 2.0 nm.

### Advantageous Effects of Invention

The fluorine-containing ether compound of the present invention is a compound represented by Formula (1) and is suitable as a material for a lubricant for a magnetic recording medium.

Since the lubricant for a magnetic recording medium of the present invention includes the fluorine-containing ether compound of the present invention, it is possible to form a lubricating layer having good chemical substance resistance and a high spin-off suppressing effect.

The magnetic recording medium of the present invention includes a lubricating layer including the fluorine-containing ether compound of the present invention. Therefore, the magnetic recording medium of the present invention has good chemical substance resistance and a high spin-off suppressing effect, and excellent reliability and durability. In addition, since the magnetic recording medium of the present invention includes the lubricating layer having good chemical substance resistance and capable of suppressing spin-off, the thickness of the lubricating layer can be reduced, and thus, the magnetic spacing can be further reduced.

### BRIEF DESCRIPTION OF DRAWINGS

FIG. 1 is a schematic cross-sectional view showing an embodiment of the magnetic recording medium of the present invention.

### DESCRIPTION OF EMBODIMENTS

The present inventors have conducted extensive research as shown below in order to accomplish the objects.

In the related art, a fluorine-containing ether compound having polar groups such as a hydroxy group at a terminal and a central part of a chain-like structure has been preferably used as a material for a lubricant for a magnetic recording medium (which may hereinafter be simply referred to as a “lubricant”) which is applied onto a surface of a protective layer. The polar groups in the fluorine-containing ether compound are bonded to active points on the protective layer to improve the adhesion of the lubricating layer to the protective layer.

However, in a case where a lubricating layer of thin thickness is formed on the protective layer, using a lubricant including the fluorine-containing ether compound in the related art, it is difficult to realize a lubricating layer having good chemical substance resistance and a high spin-off suppressing effect, as shown below.

For example, in a fluorine-containing ether compound in which divalent linking groups having only one hydroxy group are arranged among a plurality of perfluoropolyether chains, a hydroxy group contained in the divalent linking group may be hindered from being adsorbed to the protective layer and may not be able to interact with an active point on the protective layer by the bulky perfluoropolyether chains arranged on both neighboring sides.

In addition, in a fluorine-containing ether compound in which divalent linking groups having a rigid structure are arranged among a plurality of perfluoropolyether chains, even in a case where the divalent linking group has a plurality of hydroxy groups, the divalent linking group may not be able to interact with an active point on the protective layer. This is because since the divalent linking group having a rigid structure cannot move freely, it may move by the linkage with the perfluoropolyether chains arranged on both neighboring sides of the divalent linking group, and may be detached from the protective layer in a case where the perfluoropolyether chains move molecularly.

In addition, in the fluorine-containing ether compound in which divalent linking groups having a plurality of hydroxy groups, in which the hydroxy groups are in close proximity to each other, are arranged among a plurality of perfluoropolyether chains, even in a case where the fluorine-containing ether compound has a flexible structure in which two glycerin structures are directly bonded (—O—CH₂—CH(OH)—CH₂—O—CH₂—CH(OH)—CH₂—O—), the hydroxy groups included in the divalent linking group are likely to interact with each other in the molecule. Therefore, the hydroxy groups contained in the divalent linking group are not likely to be involved in the interaction with polar groups included in other fluorine-containing ether compound molecules present in the lubricating layer.

In this way, in a case where the fluorine-containing ether compound included in the lubricant includes a polar group which is not involved in the interaction with <1>an active point on the protective layer or <2>a polar group included in another fluorine-containing ether compound molecule present in the lubricating layer, a contaminant are likely to be incorporated in the vicinity of the polar group. In addition, in a case where the adhesion to the protective layer is insufficient, the lubricant is more likely to be scattered or evaporated due to centrifugal force and heat generation associated with the rotation of the magnetic recording medium. Therefore, in a case where the polar group in the fluorine-containing ether compound included in the lubricant is not likely to be involved in the interaction with <1> or <2>, the chemical substance resistance and the spin-off suppressing effect of the lubricating layer are poor.

Therefore, the present inventors have focused on the interactions between the polar group in the fluorine-containing ether compound molecule included in the lubricating layer and <1> and <2>, and have conducted extensive studies. As a result, the present inventors have found that the fluorine-containing ether compound may be a fluorine-containing ether compound in which divalent linking groups linking a plurality of perfluoropolyether chains through a methylene group (—CH₂—) have a structure represented by Formula (2) including of two glycerin structures and an acyclic saturated hydrocarbon group having an appropriate length, arranged between the two glycerin structures, in which terminal groups having a polar group are contained at both terminals of the molecule.

The divalent linking group represented by Formula (2) in such a fluorine-containing ether compound has a highly flexible structure with two glycerin structures having a hydroxy group, and the distance between the two glycerin structures is appropriate due to the acyclic saturated hydrocarbon group included in the divalent linking group. Moreover, the acyclic saturated hydrocarbon group has, for example, higher fluidity and an extremely small effect of hindering the motion of the two glycerin structures, as compared with a cyclic organic group. Therefore, it is presumed that the hydroxy groups included in the divalent linking group represented by Formula (2) can each move independently and freely, the interaction between the same fluorine-containing ether compound molecules is suppressed, and the hydroxy groups are easily involved in the interactions with <1> and <2>. The interaction between the hydroxy group included in the divalent linking group represented by Formula (2) and <1> contributes to the improvement of adhesion between the lubricating layer and the protective layer. In addition, the interaction between the hydroxy group included in the divalent linking group represented by Formula (2) and <2> is an intermolecular interaction with other fluorine-containing ether compounds, and contributes to the formation of a dense film which is not likely to separate from the protective layer. Therefore, the lubricating layer including the fluorine-containing ether compound is dense and not likely to be separated, and has high adhesion to the protective layer, and thus, spin-off is not likely to occur. In addition, the hydroxy group which is not involved in the interactions with <1> and <2> makes it not likely for a contaminant to be incorporated and the chemical substance resistance is good.

Furthermore, in the fluorine-containing ether compound, the perfluoropolyether chain is arranged between the terminal group having a polar group and the divalent linking group represented by Formula (2). Therefore, the interaction between the polar group in the terminal group and the hydroxy group in the divalent linking group in the molecule is not likely to occur the perfluoropolyether chain, and the terminal group and the linking group can interact with the protective layer each independently. That is, two or three perfluoropolyether chains have a terminal group or a linking group at both ends thereof, which interact with the protective layer. Therefore, the perfluoropolyether chain is suppressed from floating up and becoming bulky. Thus, the thickness of the lubricating layer including the fluorine-containing ether compound can be further reduced. In other words, the lubricating layer including the fluorine-containing ether compound can maintain the function as a lubricating layer even in a case where the thickness is small.

Furthermore, the present inventors have found that by using the lubricant including the fluorine-containing ether compound, it is possible to form a lubricating layer having good chemical substance resistance and a high spin-off suppressing effect, thereby leading to the present invention.

Hereinafter, preferred examples of the fluorine-containing ether compound, the lubricant for a magnetic recording medium, and the magnetic recording medium of the present invention will be described in detail. Furthermore, the present invention is not limited to embodiments shown below. In the present invention, numbers, amounts, positions, ratios, materials, configurations, the types, the orders, and the like may be modified, added, omitted, or substituted to an extent that the gist of the present invention is maintained.

### [Fluorine-Containing Ether Compound]

The fluorine-containing ether compound of the present embodiment is represented by Formula (1).

R¹—CH₂—R²[—CH₂—R³—CH₂—R²]ₓ—CH₂—R⁴ (1)
(in Formula (1), x represents an integer of 1 or 2; R² is a perfluoropolyether chain; (x+1) pieces of R²'s may be partially or entirely the same as or different from each other; R³ is a divalent linking group represented by Formula (2); in a case where x is 2, two R³'s may be the same as or different from each other; R¹ and R⁴ are each a terminal group having 1 to 50 carbon atoms, which has 1 to 4 polar groups; and R¹ and R⁴ may be the same as or different from each other)

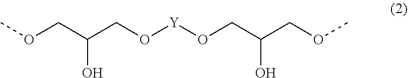

(in Formula (2), Y represents an acyclic divalent saturated hydrocarbon group 2 to 8 carbon atoms, which may have an ether oxygen atom between carbon atoms)

As shown in Formula (1), the fluorine-containing ether compound of the present embodiment has a skeleton where the divalent linking group represented by R³ and the perfluoropolyether chain (which may hereinafter be referred to as the “PFPE chain”) represented by R² are linked through a methylene group. The terminal group represented by R¹ is bonded to one end of the skeleton through a methylene group, and the terminal group represented by R⁴ is bonded to the other end of the skeleton through a methylene group.

In the fluorine-containing ether compound represented by Formula (1), x represents an integer of 1 or 2. Since x is an integer of 1 or 2 in the fluorine-containing ether compound represented by Formula (1), the number (x+1) of PFPE chains represented by R² is 2 or 3, and unlike a compound having one PFPE chain represented by R², a divalent linking group having a hydroxy group represented by R³ is arranged between adjacent R²'s. Therefore, a lubricating layer having more excellent adhesion to the protective layer and not likely to cause spin-off is obtained, as compared with a compound in which the number of the PFPE chains represented by R² is 1. In addition, the molecule is not too large and can move freely, as compared with a compound in which the number of the PFPE chains represented by R² is 4 or more. Therefore, a lubricating layer which is likely to wet-spread on the protective layer and has a thin and even film thickness is easily obtained. In addition, since x is an integer of 1 or 2, the number of R³'s arranged between R²'s is 1 or 2. Thus, the number of the hydroxy groups contained in —R²[—CH₂—R³—CH₂—R²]ₓ— in Formula (1) is likely to be appropriate, and a lubricating layer having good adhesion to the protective layer is likely to be obtained. In addition, in the fluorine-containing ether compound represented by Formula (1), as compared with a case where x is 3 or more, for example, a polar group which is not involved in the interaction with an active point on the protective layer or with a polar group included in another fluorine-containing ether compound present in the lubricating layer is not likely to be generated, incorporation of a contaminant due to the hydroxy group which is not involved in the interactions with <1> and <2>is not likely to occur, and thus, a lubricating layer having good chemical substance resistance can be formed.

### (Divalent Linking Group Represented by R³)

In the fluorine-containing ether compound represented by Formula (1), x pieces of R³'s each represent a divalent linking group represented by Formula (2). The divalent linking group represented by Formula (2) has oxygen atoms at both terminals of a chain-like structure, and the oxygen atom is bonded to the methylene group bonded to the divalent linking group represented by R³ through an ether bond. The oxygen atom included in the chain-like structure of the divalent linking group represented by Formula (2) forms an ether bond to impart moderate flexibility to the fluorine-containing ether compound represented by Formula (1), and to increase the affinity between the hydroxy group contained in the divalent linking group represented by Formula (2) and the protective layer.

The divalent linking group represented by Formula (2) includes a glycerin structure (—O—CH₂—CH(OH)—CH₂—O—) at both ends. Since the glycerin structure is flexible, the structure represented by Formula (2) in which these two glycerin structures are linked by an acyclic saturated hydrocarbon group which will be described later has extremely high flexibility. Therefore, even in a case where the perfluoropolyether chains arranged on both sides move molecularly, the two hydroxy groups in the divalent linking group represented by Formula (2) can each move independently and freely, and the interaction of the hydroxy group in the divalent linking group with both the active point on the protective layer and the polar group included in the other fluorine-containing ether compound in the lubricating layer is easily maintained. Thus, the number of the hydroxy groups in the divalent linking group, which are not involved in the interaction and which may have a contaminant incorporated, is reduced. Therefore, the lubricating layer including the fluorine-containing ether compound has excellent chemical substance resistance. In addition, since the hydroxy groups in the two glycerin structures are likely to be involved in the interaction with the protective layer or the intermolecular interaction with other fluorine-containing ether compounds, the lubricating layer including the fluorine-containing ether compound is dense, and spin-off is suppressed.

The divalent linking group represented by Formula (2) has two hydroxy groups. Since the divalent linking group represented by Formula (2) has two hydroxy groups, the lubricating layer including the fluorine-containing ether compound has good adhesiveness (adhesion) with the protective layer. That is, even in a case where one hydroxy group in Formula (2) is inhibited from being adsorbed to the protective layer due to the bulkiness of the perfluoropolyether chains on both neighboring sides, the other hydroxy group can be adsorbed to the protective layer. In addition, since the number of the hydroxy groups contained in the divalent linking group is 2, in the magnetic recording medium including the lubricating layer including the fluorine-containing ether compound, the polarity of the fluorine-containing ether compound is increased, thus causing the molecules to aggregate. Thus, the adhesion to the protective layer is decreased and the occurrence of spin-off can be prevented.

Y in the divalent linking group represented by Formula (2) is an acyclic divalent saturated hydrocarbon group having 2 to 8 carbon atoms. Since the number of carbon atoms included in Y is 2 or more, the distance between the hydroxy groups in the two glycerin structures bonded to both sides of Y is appropriately maintained. Therefore, two hydroxy groups in the divalent linking group represented by Formula (2) can each move independently. In addition, since the number of carbon atoms included in Y is 8 or less, the effect of Y becoming bulky and hindering the motion of the two glycerin structures bonded to both sides of Y is reduced. The number of carbon atoms included in Y is preferably 3 to 6. In a case where the number of carbon atoms included in Y is 3 or more, the distance between the two hydroxy groups included in the divalent linking group represented by Formula (2) is more appropriate. As a result, the hydroxy group in the divalent linking group is likely to be involved in the interaction with an active point on the protective layer and with a polar group included in other fluorine-containing ether compounds present in the lubricating layer. In addition, in a case where the number of carbon atoms included in Y is 3 or more, the affinity of the saturated hydrocarbon group with the contaminant is decreased, and thus, the lubricating layer can suppress the incorporation of the contaminant. Thus, the chemical substance resistance is better.

Y in the divalent linking group represented by Formula (2) is a saturated hydrocarbon group. Therefore, for example, the flexibility is higher and the motion of two glycerin structures bonded to both sides of Y is not likely to be hindered, as compared with a case where Y has an unsaturated bond. In addition, Y in the divalent linking group represented by Formula (2) is acyclic. Therefore, for example, the fluidity is higher and the motion of two glycerin structures bonded to both sides of Y is not likely to be hindered, as compared with a case where Y has a cyclic structure.

Y in the divalent linking group represented by Formula (2) may include an ether oxygen atom (—O—) between carbon atoms. In a case where Y includes an ether oxygen atom between carbon atoms, the flexibility is more excellent. Y may be linear or branched.

It is preferable that x pieces of R³'s each independently represent a divalent linking group represented by Formula (2-1) or (2-2).

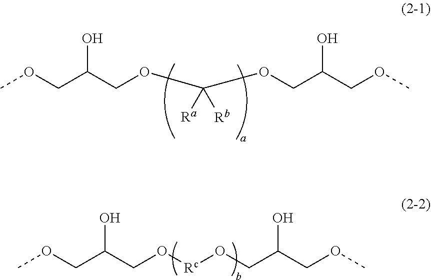

(in Formula (2-1), a represents an integer of 2 to 8; a pieces of R^{a}'s and R^{b}'s each independently represent a hydrogen atom or a methyl group; a total number of carbon atoms included in a pieces of (—CR^{a}R^{b}—)'s is 2 to 8; an oxygen atom at a left terminal of Formula (2-1) is bonded to a methylene group on an R¹ side in Formula (1), and an oxygen atom at a right terminal is bonded to a methylene group on an R⁴ side in Formula (1))
(in Formula (2-2), b represents an integer of 2 to 4; b pieces of R^{c'}s each independently represent —CH₂CH₂—, —CH₂CH₂CH₂—, —CH(CH₃)CH₂—, or —CH₂CH(CH₃)—; a total number of carbon atoms included in b pieces of R^{c}'s is 4 to 8; an oxygen atom at a left terminal of Formula (2-2) is bonded to the methylene group on the R¹ side in Formula (1), and an oxygen atom at a right terminal is bonded to the methylene group on the R⁴ side in Formula (1)).

The divalent linking group represented by Formula (2-1) is the divalent linking group represented by Formula (2), in which —O—Y—O— is replaced with —O—(—CR^{a}R^{b}—)ₐ—O—. a represents an integer of 2 to 8. a pieces of R^{a}'s and R^{b}'s each independently represent a hydrogen atom or a methyl group. In the divalent linking group represented by Formula (2-1), two glycerin structures are linked by a saturated hydrocarbon group having a total number of carbon atoms of 2 to 8, which does not include an ether oxygen atom.

Since a pieces of (—CR^{a}R^{b}—)'s do not include an ether oxygen atom, and a pieces of R^{a}'s and R^{b}'s are each independently a hydrogen atom or a methyl group, the divalent linking group represented by Formula (2-1) has low affinity with a contaminant. In a case where the number of carbon atoms included in a pieces of (—CR^{a}R^{b}—)'s is 3 or more, the fluorine-containing ether compound has further lower affinity with a contaminant and can form a lubricating layer which is not likely to incorporate the contaminant.

Since a in Formula (2-1) is 2 or more, at least 8 atoms not including an ether oxygen atom are present between the hydroxy groups included in the glycerin structures at both ends. Therefore, the distance between the hydroxy groups included in the glycerin structures at both ends is appropriate. As a result, the hydroxy groups included in the glycerin structures at both ends are suppressed from being aggregated with each other in the molecule and can each move independently. In addition, since a in Formula (2-1) is 8 or less and the total number of carbon atoms included in a pieces of (—CR^{a}R^{b}—)'s is 8 or less, the effect of the rigidity of the saturated hydrocarbon group to hinder the motion of the glycerin structures at both ends is small.

In the divalent linking group represented by Formula (2-1), the polarity in the vicinity of the glycerin structure at both ends is decreased by a saturated hydrocarbon group not including an ether oxygen atom, and thus, the affinity with a contaminant is reduced. Therefore, the lubricating layer including the fluorine-containing ether compound, in which R³ is the divalent linking group represented by Formula (2-1), has better chemical substance resistance, which is thus preferable.

The divalent linking group represented by Formula (2-2) is the divalent linking group represented by Formula (2), in which —O—Y—O— is replaced with —O—(R^{c}—O)_{b}—. b represents an integer of 2 to 4. b pieces of R's each independently represent —CH₂CH₂—, —CH₂CH₂CH₂—, —CH(CH₃)CH₂—, or —CH₂CH(CH₃)—. In the divalent linking group represented by Formula (2-2), two glycerin structures are linked by a saturated hydrocarbon group having a total number of carbon atoms of 4 to 8, which includes an ether oxygen atom.

Since b in Formula (2-2) is 2 or more, at least 11 atoms including an ether oxygen atom are present between the hydroxy groups included in the glycerin structures at both ends. Therefore, the distance between the hydroxy groups included in the glycerin structures at both ends is appropriate. As a result, the hydroxy groups included in the glycerin structures at both ends are suppressed from being aggregated with each other in the molecule and can each move independently. In addition, since b in Formula (2-2) is 4 or less, the ether oxygen atom is present between the two glycerin structures and the total number of carbon atoms included in b pieces of R's is 8 or less, the effect of the rigidity of the saturated hydrocarbon group to hinder the motion of the glycerin structures at both ends is small.

In the divalent linking group represented by Formula (2-2), since the saturated hydrocarbon group arranged between the two glycerin structures has a regular arrangement having a repeating structure consisting of an ether oxygen atom and R^{c}, the intermolecular interaction of the fluorine-containing ether compounds is improved. Therefore, the lubricating layer including the fluorine-containing ether compound, in which R³ is the divalent linking group represented by Formula (2-2), the lubricating layer is denser and spin-off is further suppressed, which is thus preferable.

In a case where x in Formula (1) is 2, two R³'s may be the same as or different from each other. In a case where the two R³'s are the same as each other, a coating state of the fluorine-containing ether compound with respect to the protective layer is more even, and a lubricating layer having better adhesion can be formed. The “two R³'s are the same as each other” means that the atoms included in the two R³'s are symmetrically arranged with respect to R² arranged at the center of the chain-like structure of the molecule.

(Terminal Groups Represented by R¹ and R⁴)

In the fluorine-containing ether compound represented by Formula (1), R¹ and R⁴ are each a terminal group having 1 to 50 carbon atoms, which has 1 to 4 polar groups.

In the present embodiment, since R¹ and R⁴ are each the terminal group, a lubricating layer including the fluorine-containing ether compound represented by Formula (1) has good chemical substance resistance and a high spin-off suppressing effect.

More specifically, since the number of the polar groups included in each of R¹ and R⁴ is 1 or more, in a case where a lubricating layer is formed on the protective layer, using a lubricant including the fluorine-containing ether compound, a suitable interaction occurs between the lubricating layer and the protective layer. As a result, the lubricating layer has excellent adhesion to the protective layer and a high spin-off suppressing effect. In addition, since the number of the polar groups included in each of R¹ and R⁴ is 4 or less, the polar groups included in R¹ and R⁴ are less likely not to be involved in the interaction with active points on the protective layer or the polar groups included in other fluorine-containing ether compounds present in the lubricating layer, and a lubricating layer having high chemical substance resistance and a high spin-off suppressing effect can be obtained. In addition, since the number of the polar groups included in each of R¹ and R⁴ is 4 or less, it is possible to suppress the fluorine-containing ether compound from being aggregated into an agglomerate due to excessively high polarity of the fluorine-containing ether compound, leading to a loss of the smoothness of the lubricating layer in the lubricating layer including the fluorine-containing ether compound. The number of the polar groups included in R¹ and R⁴ is preferably 3 or less, and most preferably 2 since the fluorine-containing ether compound can form a lubricating layer having higher chemical substance resistance and a higher spin-off suppressing effect. In a case where the number of the polar groups included in R¹ and R⁴ is 2, the polar groups contained in the terminal groups represented by R¹ and R⁴ can be effectively suppressed from being aggregated with each other in a magnetic recording medium having a lubricating layer including the fluorine-containing ether compound. In addition, the polar groups included in R¹ and R⁴ are even less likely not to be involved in the interaction with the active point on the protective layer or the polar groups included in other fluorine-containing ether compounds present in the lubricating layer. As a result, the fluorine-containing ether compound can prevent the contaminant from being incorporated into the magnetic recording medium, and can form a lubricating layer having higher chemical substance resistance and a higher spin-off suppressing effect.

R¹ and R⁴ may be the same as or different from each other. Therefore, the number of the polar groups contained in R¹ and the number of the polar groups contained in R⁴ may be the same as or different from each other. The number of the polar groups contained in R¹ and the number of the polar groups contained in R⁴ are preferably the same as each other since the coating state of the fluorine-containing ether compound with respect to the protective layer is more even and a lubricating layer having better adhesion can be formed.

The total number of the polar groups contained in R¹ and the polar groups contained in R⁴ in Formula (1) is preferably 2 to 6, more preferably 3 to 6, and still more preferably 4 to 6. In a case where the total number of the polar groups is 2 or more, the interaction between the polar group contained in R¹ and R⁴ in the fluorine-containing ether compound and the protective layer is stronger. Thus, the fluorine-containing ether compound can form a lubricating layer having high adhesion to the protective layer. As a result, the lubricating layer formed of the fluorine-containing ether compound has excellent chemical substance resistance. In a case where the total number of the polar groups is 6 or less, the polarity of the fluorine-containing ether compound is not excessively increased and the affinity with the contaminant can be maintained low. Therefore, the fluorine-containing ether compound can form a lubricating layer having more excellent chemical substance resistance.

R¹ and R⁴ each have 1 to 50 carbon atoms, and preferably have 3 to 20 carbon atoms, and more preferably have 4 to 15 carbon atoms. In a case where the number of carbon atoms is 1 or more, the affinity with a contaminant is decreased, and therefore, the lubricating layer has good chemical substance resistance. In a case where the number of carbon atoms is 50 or less, the lubricating layer has flexible structures at terminal group portions, and thus, the adhesion of the fluorine-containing ether compound is improved and the lubricating layer can suppress spin-off.

R¹ and R⁴ can be appropriately selected according to the performance required for the lubricant including the fluorine-containing ether compound. The one to four polar groups contained in each of R¹ and R⁴ may be partially or entirely the same as or different from each other.

The polar groups contained in R¹ and R⁴ are each preferably at least one selected from the group consisting of a hydroxy group (—OH), an amino group (—NH₂), a carboxy group (—COOH), a formyl group (—(C═O)H), a carbonyl group (—CO—), a sulfo group (—SO₃H), a cyano group (—CN), and a group having an amide bond (—NR⁷COR⁸ or —CONR⁹R¹⁰; R⁷, R⁸, R⁹, and R¹⁰ are each independently a hydrogen atom or an organic group). As the group having an amide bond, as shown in the formula, both a group (for example, a carboxamide group (—C(═O)NH₂)) bonded to a carbon atom constituting the amide bond and a group (for example, an acetamide group (—NHC(═O)CH₃)) bonded to a nitrogen atom constituting the amide bond are included. In the group having an amide bond, R⁷ and R⁸ may be bonded to each other to form a ring, and R⁹ and R¹⁰ may be bonded to each other to form a ring. R⁷, R⁸, R⁹, and R¹⁰ in the group having an amide bond are each independently preferably selected from the group consisting of a hydrogen atom, a methyl group, an ethyl group, a propyl group, and a butyl group.

It is preferable that the polar groups contained in R¹ and R⁴ are each independently at least one selected from the group consisting of a hydroxy group, a cyano group, and a group having an amide bond. A reason therefor is that the hydroxy group, the cyano group, and the group having an amide bond are chemically stable, and a lubricating layer including a fluorine-containing ether compound having the polar group is not deteriorated for a long period of time. In addition, another reason is that the hydroxy group, the cyano group, and the group having an amide bond have an acidity which is not excessively high and have almost no effect on the corrosion of a substrate.

It is more preferable that R¹ and R⁴ each include at least one hydroxy group as the polar group. By configuring R¹ and R⁴ to include a hydroxy group, in a case where a lubricant including a fluorine-containing ether compound is used to form a lubricating layer on the protective layer, a suitable interaction occurs between the lubricating layer and the protective layer. As a result, the lubricating layer has excellent adhesion to the protective layer and a high spin-off suppressing effect.

It is more preferable that all of the polar groups contained in R¹ and R⁴ are hydroxy groups. In a case where all the polar groups contained in R¹ and R⁴ are hydroxy groups, a coating state of the fluorine-containing ether compound with respect to the protective layer can be made more even.

In the fluorine-containing ether compound represented by Formula (1), it is preferable that R¹ and R⁴ are each independently a terminal group represented by Formula (3).

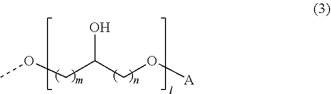

(in Formula (3), 1 represents an integer of 1 to 3; 1 pieces of m's each independently represent an integer of 1 to 6; 1 pieces of n's each independently represent an integer of 1 to 6; at least one of m and n is 1 in one repeating unit; and A represents an alkyl group which may have a polar group, an organic group including a carbon-carbon unsaturated bond, which may have a polar group, or a hydrogen atom)

In a case where R¹ and R⁴ are each the terminal group represented by Formula (3), an oxygen atom is bonded to the methylene group (—CH₂—) bonded to R². That is, R¹ and R⁴ have an oxygen atom at an end part on the side bonded to CH₂ each adjacent to R¹ and R⁴. Oxygen atoms arranged at the end parts of R¹ and R⁴ form an ether bond (—O—) with atoms bonded to both sides thereof. This ether bond imparts moderate flexibility to the fluorine-containing ether compound represented by Formula (1), and increases the affinity between the polar group contained in the terminal group represented by R¹ and R⁴ and the protective layer. Thus, the fluorine-containing ether compound represented by Formula (1) can form a lubricating layer having excellent adhesion to the protective layer.

1 in Formula (3) is an integer of 1 to 3, preferably an integer of 1 or 2, and most preferably 1. In a case where 1 in Formula (3) is 3 or less, there are too many hydroxy groups in the terminal group represented by Formula (3). Thus, the affinity with a contaminant can be prevented from being increased and a lubricating layer having good chemical substance resistance can be obtained.

In a case where 1 in Formula (3) is 2 or 3, combinations of m's and n's in the two or three repeating units (—(CH₂)ₘ—CH(OH)—(CH₂)ₙ—O—) may be different from each other, or may be partially or entirely the same as each other.

1 pieces of m's in Formula (3) each independently represent an integer of 1 to 6 and I pieces of n's each independently represent an integer of 1 to 6.

In one repeating unit (—(CH₂)ₘ—CH(OH)—(CH₂)ₙ—O—) in Formula (3), at least one of m and n is 1. This is because the motion of the hydroxy group in the repeating unit is not decreased since the number of carbon atoms in the alkylene group between the carbon atom to which a hydroxy group is bonded and the ether oxygen atom is too large.

A in Formula (3) represents an alkyl group which may have a polar group, an organic group which may have a polar group, or a hydrogen atom, the organic group including a carbon-carbon unsaturated bond.

In a case where A in Formula (3) is an alkyl group having no polar group, examples of A include a methyl group, an ethyl group, a propyl group, a butyl group, a pentyl group, and a hexyl group.

In a case where A in Formula (3) is an alkyl group having a polar group, the polar groups mentioned as preferred examples of the polar group included in R¹ and R⁴ can be used as the polar group. Among these polar groups, at least one selected from the group consisting of the hydroxy group, the cyano group and the group having an amide bond is more preferable. The hydroxy group, the cyano group or the group having an amide bond is preferable since the lubricating layer including the fluorine-containing ether compound having these polar groups is chemically stable and is not deteriorated for a long period of time. In addition, another reason is that the hydroxy group, the cyano group, and the group having an amide bond have an acidity which is not excessively high and have almost no effect on the corrosion of a substrate.

In a case where A in Formula (3) is an alkyl group having a polar group, examples of A include a 2-hydroxyethyl group, a 3-hydroxypropyl group, a 4-hydroxybutyl group, a 5-hydroxypentyl group, a 6-hydroxyhexyl group, a 2-aminoethyl group, a 3-aminopropyl group, a 2-carboxyethyl group, a 3-carboxypropyl group, a 2-carbonyl ethyl group, a 3-carbonyl propyl group, a 2-acetylethyl group, a 3-acetylpropyl group, a 2-sulfoethyl group, a 3-sulfopropyl group, a 2-cyanoethyl group, a 3-cyanopropyl group, a 4-cyanobutyl group, a 2-acetamidoethyl group, a 3-acetamidopropyl group, a 4-acetamidobutyl group, a 2-carboxamidoethyl group, a 3-carboxamidopropyl group, and a 4-carboxamidobutyl group.

Among those, any of the 2-hydroxyethyl group, the 3-hydroxypropyl group, the 4-hydroxybutyl group, the 5-hydroxypentyl group, the 6-hydroxyhexyl group, the 2-cyanoethyl group, the 3-cyanopropyl group, the 2-acetamidoethyl group, the 2-carboxamidoethyl group, and the 3-carboxamidopropyl group is preferable, and any of the 2-hydroxyethyl group, the 3-hydroxypropyl group, the 2-cyanoethyl group, the 3-cyanopropyl group, and the 2-acetamidoethyl group is more preferable.

In a case where A in Formula (3) is an organic group including a carbon-carbon unsaturated bond, examples of A include an organic group including at least one selected from the group consisting of an aromatic hydrocarbon, an unsaturated heterocyclic ring, an alkenyl group, and an alkynyl group. In the functional groups (active points) present in a large number on the protective layer, a locally charged site and a site having charges distributed widely are each present. R³ represented by Formula (2) and the hydroxy group included in Formula (3) exhibit an adsorption ability by allowing the hydrogen atom to interact with a locally charged site on the protective layer through a hydrogen bond. On the other hand, in a case where the organic group represented by A included in Formula (3) is an organic group including an aromatic hydrocarbon, an unsaturated heterocyclic ring, an alkenyl group, or an alkynyl group, these organic groups have a delocalized charge, and thus, exhibit an adsorption ability by interacting with a site having charges distributed widely on the protective layer. Therefore, R³ represented by Formula (2) and the hydroxy group included in Formula (3), and an organic group including a carbon-carbon unsaturated bond, represented by A, included in Formula (3) are each adsorbed to different sites on the protective layer. That is, R³ represented by Formula (2) and the hydroxy group included in Formula (3), and the organic group including a carbon-carbon unsaturated bond, represented by A, included in Formula (3) can each independently interact with a functional group (active point) on the protective layer. As a result, in a case where R¹ and/or R⁴ is the terminal group represented by Formula (3) and A in Formula (3) is the organic group including a carbon-carbon unsaturated bond, the lubricating layer including the fluorine-containing ether compound has excellent adhesion to the protective layer.

In a case where A in Formula (3) is an organic group including a carbon-carbon unsaturated bond having no polar group, examples of A include a phenyl group, a methoxyphenyl group, a fluorinated phenyl group, a naphthyl group, a phenethyl group, a methoxyphenethyl group, a fluorinated phenethyl group, a benzyl group, a methoxybenzyl group, a naphthylmethyl group, a methoxynaphthyl group, a pyrrolyl group, a pyrazolyl group, a methylpyrazolylmethyl group, an imidazolyl group, a furyl group, a furfuryl group, an oxazolyl group, an isoxazolyl group, a thienyl group, a thienylethyl group, a thiazolyl group, a methylthiazolylethyl group, an isothiazolyl group, a pyridyl group, a pyrimidinyl group, a pyridazinyl group, a pyrazinyl group, an indolinyl group, a benzofuranyl group, a benzothienyl group, a benzimidazolyl group, a benzoxazolyl group, a benzothiazolyl group, a benzopyrazolyl group, a benzoisoxazolyl group, a benzoisothiazolyl group, a quinolyl group, an isoquinolyl group, a quinazolinyl group, a quinoxalinyl group, a phthalazinyl group, a cinnolinyl group, a vinyl group, an allyl group, a butenyl group, a propynyl group, a propargyl group, a butynyl group, a methylbutynyl group, a pentynyl group, a methylpentynyl group, and a hexynyl group.

Among those, any of the phenyl group, the methoxyphenyl group, the naphthyl group, the phenethyl group, the methoxyphenethyl group, the fluorinated phenethyl group, the thienylethyl group, the allyl group, the butenyl group, and the propargyl group is preferable, and any one of the phenyl group, the methoxyphenyl group, the allyl group, and the butenyl group is more preferable. In a case where A is any of the phenyl group, the methoxyphenyl group, the allyl group, and the butenyl group, the interaction with the site having charges distributed widely on the protective layer can be stronger, and the adhesion to the protective layer can be improved.

In a case where A in Formula (3) is an organic group including a carbon-carbon unsaturated bond having a polar group, as the polar group, the polar group mentioned as preferred examples of the polar group included in R¹ and R⁴ can be used. Among these polar groups, at least one selected from the group consisting of the cyano group and the group having an amide bond is more preferable. The cyano group or the group having an amide bond is preferable since the lubricating layer including the fluorine-containing ether compound having these polar groups is chemically stable and is not deteriorated for a long period of time. In addition, another reason is that the cyano group and the group having an amide bond have an acidity which is not excessively high and have almost no effect on the corrosion of a substrate.

In a case where A in Formula (3) is an organic group including a carbon-carbon unsaturated bond having a polar group, examples of A include a cyanophenyl group, a carboxamidophenyl group, an acetamidophenyl group, a cyanonaphthyl group, a carboxamidonaphthyl group, an acetamidonaphthyl group, a cyanophenethyl group, a carboxamidophenethyl group, an acetamidophenethyl group, a cyanobenzyl group, a carboxamidobenzyl group, and an acetamidobenzyl group.

Among those, any of the cyanophenyl group, the carboxamidophenyl group, the acetamidophenyl group, the cyanonaphthyl group, the carboxamidonaphthyl group, the acetamidonaphthyl group, the cyanobenzyl group, the carboxamidobenzyl group, and the acetamidobenzyl group is preferable, and any of the cyanophenyl group, the carboxamidophenyl group, and the acetamidophenyl group is more preferable.

In a case where A in Formula (3) is a hydrogen atom, A forms a hydroxy group with the oxygen atom in Formula (3). In a case where A is a hydrogen atom, it is preferable that n is 2 or more in the repeating unit (—(CH₂)ₘ—CH(OH)—(CH₂)ₙ—O—) in Formula (3), to which A is bonded. In a case where n is 2 or more in the repeating unit to which A is bonded, the distance between the terminal hydroxy group and a hydroxy group adjacent to the terminal hydroxy group is more appropriate, and the adhesion to the protective layer can be improved.

In the fluorine-containing ether compound represented by Formula (1), it is more preferable that R¹ and R⁴ are each independently a terminal group represented by Formula (3-1) or (3-2).

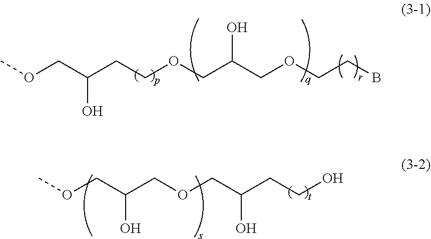

(in Formula (3-1), p represents an integer of 0 to 3, q represents an integer of 0 to 2, and r represents an integer of 1 to 5; a total value of p and r is 1 to 5; and B represents a polar group).
(in Formula (3-2), s represents an integer of 0 to 2, and t represents an integer of 1 to 5).

Each polar group included in the terminal groups represented by Formulae (3-1) and (3-2) is bonded to carbon atoms that are different from each other. In Formulae (3-1) and (3-2), carbon atoms to which the polar groups are bonded are bonded to each other through a linking group including a carbon atom to which the polar group is not bonded. Therefore, in a case where R¹ and/or R⁴ is the terminal group represented by Formula (3-1) or Formula (3-2), the linking group including a carbon atom not bonded to a polar group allows both the terminal polar group and a hydroxy group adjacent to the terminal polar group to be aligned such that the groups can adhere to the protective layer, and thus, it is presumed that the lubricating layer including the fluorine-containing ether compound represented by Formula (1) can suppress spin-off.

In the terminal group represented by Formula (3-1), a linking group between a carbon atom to which a polar group B arranged at a terminal is bonded and a carbon atom to which a hydroxy group adjacent to the polar group B arranged at the terminal is bonded includes an oxygen atom that forms an ether bond. p in Formula (3-1) represents an integer of 0 to 3, r represents an integer of 1 to 5, and a total value of p and r is 1 to 5. Therefore, the linking group has a linear chain-like structure consisting of 3 to 7 atoms including carbon atoms to which the polar group B and a hydroxy group are not bonded.

In the terminal group represented by Formula (3-1), since the linking group has a linear chain-like structure including an oxygen atom that forms an ether bond, and consisting of 3 or more atoms including carbon atoms to which the polar group B and the hydroxy group are not bonded, the distance between the polar group B and a hydroxy group adjacent to the polar group B is appropriate. Therefore, it is possible to suppress the polar group B and a hydroxy group adjacent to the polar group B from interacting with each other in the molecule, and both the polar group B and a hydroxy group adjacent to the polar group B can adhere to the protective layer. In addition, since the linking group has a linear chain-like structure consisting of 3 or more atoms, the molecular motion is appropriate, the intramolecular aggregation is not likely to occur, and the linking group has excellent adhesion to the protective layer even in a case where the linking group includes an oxygen atom that forms an ether bond.

In the terminal group represented by Formula (3-1), since the linking group having a linear chain-like structure including an oxygen atom that forms an ether bond, and consisting of 7 or less atoms including carbon atoms to which the polar group B and the hydroxy group are not bonded, the effect of the linking group becoming bulky to hinder the motion of the polar group is small. Thus, the lubricating layer including the fluorine-containing ether compound having the terminal group represented by Formula (3-1), in which the linking group has a linear chain-like structure consisting of 3 to 7 atoms including carbon atoms to which the polar group B and the hydroxy group are not bonded, has excellent adhesion to the protective layer, exhibits high chemical substance resistance, and has a high spin-off suppressing effect.

In Formula (3-1), a total value of p and r is 1 to 5, and preferably 1 to 3. In Formula (3-1), the carbon atoms included in the linking group arranged between carbon atoms to which the polar groups are bonded prevent an intramolecular interaction between the polar groups in close proximity to each other from occurring preferentially to the interaction between the polar group and the protective layer, and improves the adhesion between the polar group and the protective layer in Formula (3-1). On the other hand, in a case where the number of carbon atoms included in the linking group is excessively large, the flexibility of the terminal group represented by Formula (3-1) is decreased and it may be difficult to evenly coat the protective layer. In the terminal group represented by Formula (3-1), a total value of p and r is 5 or less, and thus, the alkylene chain of the main chain portion of Formula (3-1) is not too long. Therefore, it is prevented that due to the long rigid alkylene chain, the flexibility of the terminal portion is decreased and the interaction with the protective layer is weakened, causing the terminal portion to float up. p is preferably 0 or 1, and more preferably 0. r is preferably 1 or 2, and more preferably 1.

B in Formula (3-1) represents a polar group. As B, the polar groups mentioned as preferred examples of B included in R¹ and R⁴ can be used. Among the polar groups, B is more preferably a polar group selected from the group consisting of the hydroxy group, the cyano group, and the group having an amide bond. A reason therefor is that the hydroxy group, the cyano group, and the group having an amide bond are chemically stable, and a lubricating layer including a fluorine-containing ether compound having the polar group is not deteriorated for a long period of time. In addition, another reason is that the hydroxy group, the cyano group, and the group having an amide bond have an acidity which is not too high and have almost no effect on the corrosion of a substrate.

In Formula (3-1), q represents an integer of 0 to 2. The number of polar groups in Formula (3-1) is q+2, and as described above, the number of the polar groups included in R¹ and R⁴ is preferably 3 or less, and most preferably 2. Therefore, q in Formula (3-1) is preferably 0 or 1, and more preferably 0.

In the terminal group represented by Formula (3-2), the linking group between a carbon atom to which the terminal hydroxy group is bonded and a carbon atom to which a hydroxy group adjacent to the terminal hydroxy group is bonded does not include an oxygen atom. Therefore, the intramolecular interaction is small and the intramolecular aggregation is not likely to occur, resulting in excellent adhesion to the protective layer. t in Formula (3-2) represents an integer of 1 to 5. Therefore, the linking group has a linear chain-like structure consisting of 1 to 5 atoms including carbon atoms to which hydroxy groups are not bonded. Since the linking group included in Formula (3-2) has a linear chain-like structure consisting of one or more atoms including carbon atoms to which hydroxy groups are not bonded, the distance between the terminal hydroxy group and a hydroxy group adjacent to the terminal hydroxy group is appropriate. Therefore, it is possible to suppress the interaction between the terminal hydroxy group and a hydroxy group adjacent to the terminal hydroxy group in the molecule, and the intramolecular aggregation is not likely to occur.

In addition, in the same manner as in Formula (3-1), in Formula (3-2), in a case where the number of carbon atoms included in the linking group arranged between carbon atoms to which the polar groups are bonded is too large, the flexibility of the terminal group represented by Formula (3-2) is decreased, and thus, it may be difficult to evenly coat the entire surface of the protective layer. In the terminal group represented by Formula (3-2), t is 5 or less, and thus, the alkylene chain in the main chain portion of Formula (3-2) is not too long. Therefore, it can be prevented that due to the long rigid alkylene chain, the flexibility of the terminal portion is decreased, and the interaction between the terminal hydroxy group and the protective layer is decreased. t is preferably 1 or 2, and more preferably 1.

In addition, since the linking group having a linear chain-like structure not including an oxygen atom that forms an ether bond, and consisting of 5 or less atoms including carbon atoms to which the hydroxy groups are not bonded, the effect of the linking group becoming excessively bulky to hinder the motion of the hydroxy group is small.

Thus, the lubricating layer including the fluorine-containing ether compound, in which the linking group has a linear chain-like structure consisting of 1 to 5 atoms, not including an oxygen atom that forms an ether bond and including carbon atoms to which hydroxy groups are not bonded, has excellent adhesion to the protective layer, has excellent chemical substance resistance, and has a high spin-off suppressing effect.

In Formula (3-2), s represents an integer of 0 to 2. The number of polar groups in Formula (3-2) is s+2, and as described above, the number of the polar groups included in R¹ and R⁴ is preferably 3 or less, and most preferably 2. Therefore, s in Formula (3-2) is preferably 0 or 1, and more preferably 0.

In a case where x in Formula (1) is 2, two R³'s are present in the fluorine-containing ether compound. Since R³'s represented by Formula (2) each have two hydroxy groups, in a case where x is 2, the proportion of the number of polar groups in R¹ and R⁴ with respect to the total number of polar groups in the fluorine-containing ether compound is reduced. Since the number of active points in the protective layer is limited, in a case where the proportion of the polar groups in R¹ and R⁴ is reduced, the interaction between R¹ and R⁴ and the protective layer is weakened. Therefore, in a case where x is 2, it is preferable that R¹ and R⁴ are each Formula (3-1) or Formula (3-2) having a structure in which the interaction with the protective layer is relatively strong. In a case where R¹ and R⁴ are each Formula (3-1) or Formula (3-2), for example, in R¹ and/or R⁴, the hydroxy groups in the molecule are not likely to be aggregated and the interaction with the protective layer is likely to occur, as compared with a case where the carbon atoms to which the hydroxy groups are bonded are directly linked to each other. Thus, in a case where R¹ and R⁴ are each Formula (3-1) or Formula (3-2), even in a case where x is 2, the terminal portion of the fluorine-containing ether compound is not likely to float up and the adhesion to the protective layer is not likely to be decreased.

### (PFPE Chain Represented by R²)

In the fluorine-containing ether compound represented by Formula (1), R² is a perfluoropolyether chain. In a case where the lubricant including the fluorine-containing ether compound of the present embodiment is applied onto the protective layer to form a lubricating layer, the PFPE chain represented by R² coats a surface of the protective layer and imparts lubricity to the lubricating layer, thereby reducing a frictional force between the magnetic head and the protective layer. The PFPE chain represented by R² is appropriately selected according to the performance required for the lubricant including the fluorine-containing ether compound.

(x+1) pieces of R²'s may be partially or entirely the same as or different from each other. It is preferable that (x+1) pieces of R²'s are entirely the same. This is because a coating state of the fluorine-containing ether compound with respect to the protective layer is more even, and the lubricating layer has better adhesion. The expression, two or more R²'s among (x+1) pieces of R²'s being the same, means that (x+1) pieces of R²'s include two or more R²'s having the same structure of the repeating units of the PFPE chain. The same R²'s may also include repeating units having the same structure but different average degrees of polymerization.

Examples of the PFPE chain represented by R² include a polymer or copolymer of perfluoroalkylene oxide. Examples of the perfluoroalkylene oxide include perfluoromethylene oxide, perfluoroethylene oxide, perfluoro-n-propylene oxide, perfluoroisopropylene oxide, and perfluorobutylene oxide.

It is preferable that (x+1) pieces of R²'s in Formula (1) are each independently, for example, a PFPE chain represented by Formula (4) derived from a polymer or copolymer of perfluoroalkylene oxide.

—(CF₂)_{w1}—O—(CF₂O)_{w2}—(CF₂CF₂O)_{w3}—(CF₂CF₂CF₂O)_{w4}—(CF₂CF₂CF₂CF₂O)_{w5}—(CF₂)_{w6}— (4)
(in Formula (4), w2, w3, w4, and w5 each indicate an average degree of polymerization and each independently represent 0 to 20, provided that all of w2, w3, w4, and w5 are not 0 at the same time; w1 and w6 each indicate an average value of the number of CF₂'s and each independently represent 1 to 3; and (CF₂O), (CF₂CF₂O), (CF₂CF₂CF₂O), and (CF₂CF₂CF₂CF₂O), which are repeating units in Formula (4) are not particularly limited in the sequence order)

In Formula (4), w2, w3, w4, and w5 indicate an average degree of polymerization, and each independently represent 0 to 20, preferably 0 to 15, and more preferably 0 to 10.

In Formula (4), w1 and w6 are average values indicating the number of CF₂'s and each independently represent 1 to 3. w1 and w6 are determined depending on the structure of the repeating unit arranged at the end part of the chain-like structure in the PFPE chain represented by Formula (4) or the like.

(CF₂O), (CF₂CF₂O), (CF₂CF₂CF₂O), and (CF₂CF₂CF₂CF₂O) in Formula (4) are repeating units. The repeating units in Formula (4) is not particularly limited in the sequence order. In addition, the number of kinds of repeating units in Formula (4) is also not particularly limited.

(x+1) pieces of R²'s in Formula (1) are each independently preferably any one selected from PFPE chains represented by Formulae (4-1) to (4-4).

In a case where (x+1) pieces of R²'s are each any one selected from the PFPE chains represented by Formulae (4-1) to (4-4), the fluorine-containing ether compound makes it possible to obtain a lubricating layer having good lubricity. In addition, in a case where (x+1) pieces of R²'s are each any one selected from the PFPE chains represented by Formulae (4-1) to (4-4), a proportion of the number of oxygen atoms (number of ether bonds (—O—)) to the number of carbon atoms in the PFPE chain is appropriate. Therefore, the fluorine-containing ether compound has moderate hardness. Therefore, the fluorine-containing ether compound applied onto the protective layer is not likely to be aggregated on the protective layer, and a lubricating layer of even thinner thickness can be formed on the protective layer with a sufficient coating rate. The lubricating layer formed of the fluorine-containing ether compound in which (x+1) pieces of R²'s are any one selected from the PFPE chains represented by Formulae (4-1) to (4-4) is denser, and spin-off can be further suppressed.

—CF₂—(OCF₂CF₂)ₕ—(OCF₂)ᵢ—OCF₂— (4-1)
(in Formula (4-1), h and i each indicate an average degree of polymerization, h represents 1 to 20, and i represents 0 to 20)

—CF₂CF₂—(OCF₂CF₂CF₂)ⱼ—OCF₂CF₂— (4-2)
(in Formula (4-2), j indicates an average degree of polymerization and represents 1 to 15)

—CF₂CF₂CF₂—(OCF₂CF₂CF₂CF₂)ₖ—OCF₂CF₂CF₂— (4-3)
(in Formula (4-3), k indicates an average degree of polymerization and represents 1 to 10)

—(CF₂)_{w7}—O—(CF₂CF₂CF₂O)_{w8}—(CF₂CF₂O)_{W9}—(CF₂)_{w10}— (4-4)
(in Formula (4-4), w8 and w9 each indicate an average degree of polymerization and each independently represent 1 to 20; and w7 and w10 are each an average value indicating the number of CF₂'s and each independently represent 1 or 2)

In Formula (4-1), (OCF₂CF₂) and (OCF₂) which are the repeating units is not particularly limited in the sequence order. In Formula (4-1), the number h of (OCF₂CF₂)'s and the number i of (OCF₂)'s may be the same as or different from each other. The PFPE chain represented by Formula (4-1) may be a polymer of (OCF₂CF₂). In addition, the PFPE chain represented by Formula (4-1) may be any of a random copolymer, a block copolymer, or an alternating copolymer consisting of (OCF₂CF₂) and (OCF₂).

In Formulae (4-1) to (4-3), h indicating the average degree of polymerization is 1 to 20, i is 0 to 20, j is 1 to 15, and k is 1 to 10, and thus, the fluorine-containing ether compound makes it possible to obtain a lubricating layer having good lubricity. In addition, in Formulae (4-1) to (4-3), h and i each indicating an average degree of polymerization are 20 or less, j is 15 or less, and k is 10 or less, and thus, the viscosity of the fluorine-containing ether compound is not excessively high, and a lubricant including the fluorine-containing ether compound is easy to apply, which is thus preferable. h, i, j, and k, each indicating an average degree of polymerization, are each preferably 1 to 10, more preferably 1.5 to 8, and still more preferably 2 to 7 since the fluorine-containing ether compound is likely to wet-spread on the protective layer and makes it possible to obtain a lubricating layer having an even film thickness.

In Formula (4-4), (CF₂CF₂CF₂O) and (CF₂CF₂O), which are the repeating units, is not particularly limited in the sequence order. In Formula (4-4), the number w8 of (CF₂CF₂CF₂O)'s and the number w9 of (CF₂CF₂O)'s, each indicating an average degree of polymerization, may be the same as or different from each other. Formula (4-4) may include any of a random copolymer, a block copolymer, and an alternating copolymer, each consisting of monomer units (CF₂CF₂CF₂O) and (CF₂CF₂O).

In Formula (4-4), w8 and w9, each indicating an average degree of polymerization, are each independently 1 to 20, preferably 1 to 15, and more preferably 1 to 10. w7 and w10 in Formula (4-4) are average values indicating the number of CF₂'s and each independently represent 1 or 2. w7 and w10 are determined depending on the structure of the repeating unit arranged at an end part of the chain-like structure in the PFPE chain represented by Formula (4-4) or the like.

In the fluorine-containing ether compound represented by Formula (1), in a case where x is 1, it is preferable that two R²'s are the same as each other and R¹ and R⁴ are the same as each other. This is because it is possible to produce the fluorine-containing ether compound easily and efficiently.

In the fluorine-containing ether compound represented by Formula (1), in a case where x is 2, it is preferable that two R³'s are the same as each other, R² on the R¹ side and R² on the R⁴ side are the same as each other, and R¹ and R⁴ are the same as each other. This is because it is possible to produce the fluorine-containing ether compound easily and efficiently.

Specifically, the fluorine-containing ether compound represented by Formula (1) is preferably any of compounds represented by Formulae (AA) to (AJ), (BA) to (BR), (CA) to (CF), and (DA) to (DG).

In a case where the compound represented by Formula (1) is any of the compounds represented by Formulae (AA) to (AJ), (BA) to (BR), (CA) to (CF), and (DA) to (DG), it is possible to form a lubricating layer in which raw materials therefor are easily available and the lubricating layer has better chemical substance resistance and a high spin-off suppressing effect on a magnetic recording medium even in a case where the thickness is small.

Furthermore, the compounds represented by Formulae (AA) to (AJ), (BA) to (BR), (CA) to (CF), and (DA) to (DG), Rf₁, Rf₂, and Rf₃ representing a PFPE chain each have the following structures. That is, in the compounds represented by Formulae (AA) to (AJ), (BC) to (BR), (CA) to (CF), and (DB) to (DG), Rf₁ is the PFPE chain represented by Formula (4-1). In the compounds represented by Formulae (BA) and (DA), Rf₂ is the PFPE chain represented by Formula (4-2). In the compound represented by Formula (BB), Rf₃ is the PFPE chain represented by Formula (4-3). Furthermore, h and i in Rf₁, j in Rf₂, and k in Rf₃, representing the PFPE chain in Formulae (AA) to (AJ), (BA) to (BR), (CA) to (CF), and (DA) to (DG), are each a value indicating the average degree of polymerization, and are thus not necessarily an integer.

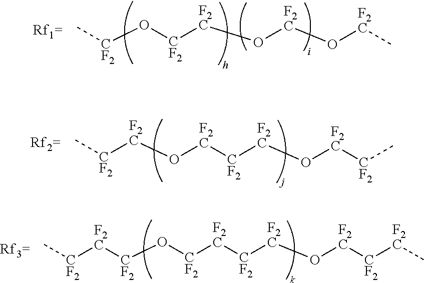

In the compounds represented by Formulae (AA) to (AJ), (BA) to (BR), (CA) to (CF), and (DA) to (DG), R³ is the linking group represented by Formula (2-1) or (2-2).

In the compounds represented by Formulae (AA) to (AJ), (BA) to (BR), (CA) to (CF), and (DA) to (DG), both R¹ and R⁴ are the terminal groups represented by Formula (3), (3-1), or (3-2).

In the compounds represented by Formulae (AA) to (AG), x in Formula (1) is 1. R³ is the linking group represented by Formula (2-1). R¹ and R⁴ are each the terminal group represented by Formula (3-1). R² is the PFPE chain represented by Formula (4-1).

In the compounds represented by Formulae (AH) to (AJ), x in Formula (1) is 1. R³ is the linking group represented by Formula (2-2). R¹ and R⁴ are each the terminal group represented by Formula (3-1). R² is the PFPE chain represented by Formula (4-1).

In the compounds represented by Formulae (BA) and (BB), x in Formula (1) is 1. R³ is the linking group represented by Formula (2-1). R¹ and R⁴ are each the terminal group represented by Formula (3-1). In Formula (BA), R² is the PFPE chain represented by Formula (4-2), and in Formula (BB), R² is the PFPE chain represented by Formula (4-3).

In the compounds represented by Formulae (BC) to (BR), x in Formula (1) is 1. R³ is the linking group represented by Formula (2-1). In Formulae (BC), (BE), (BF), (BH) to (BJ), (BL), (BQ), and (BR), R¹ and R⁴ are each a terminal group represented by Formula (3-1); in Formulae (BD), (BG), and (BK), R¹ and R⁴ are each a terminal group represented by Formula (3-2); and in Formulae (BM) to (BP), R¹ and R⁴ are each a terminal group represented by Formula (3) other than Formulae (3-1) and (3-2). R² is the PFPE chain represented by Formula (4-1).

In the compounds represented by Formulae (CA) to (CE), x in Formula (1) is 2. R³ is the linking group represented by Formula (2-1). R¹ and R⁴ are each the terminal group represented by Formula (3-1). R² is the PFPE chain represented by Formula (4-1).

In the compound represented by Formula (CF), x in Formula (1) is 2. R³ is the linking group represented by Formula (2-2). R¹ and R⁴ are each the terminal group represented by Formula (3-1). R² is the PFPE chain represented by Formula (4-1).

In the compound represented by Formula (DA), x in Formula (1) is 2. R³ is the linking group represented by Formula (2-1). R¹ and R⁴ are each the terminal group represented by Formula (3-1). R² is the PFPE chain represented by Formula (4-2).

In the compounds represented by Formulae (DB) to (DG), x in Formula (1) is 2. R³ is the linking group represented by Formula (2-1). In Formulae (DB), and (DD) to (DF), R¹ and R⁴ are each a terminal group represented by Formula (3-1); in Formula (DC), R¹ and R⁴ are each a terminal group represented by Formula (3-2); and in Formula (DG), R¹ and R⁴ are each a terminal group represented by Formula (3) other than Formulae (3-1) and (3-2). R² is the PEPE chain represented by Formula (4-1).

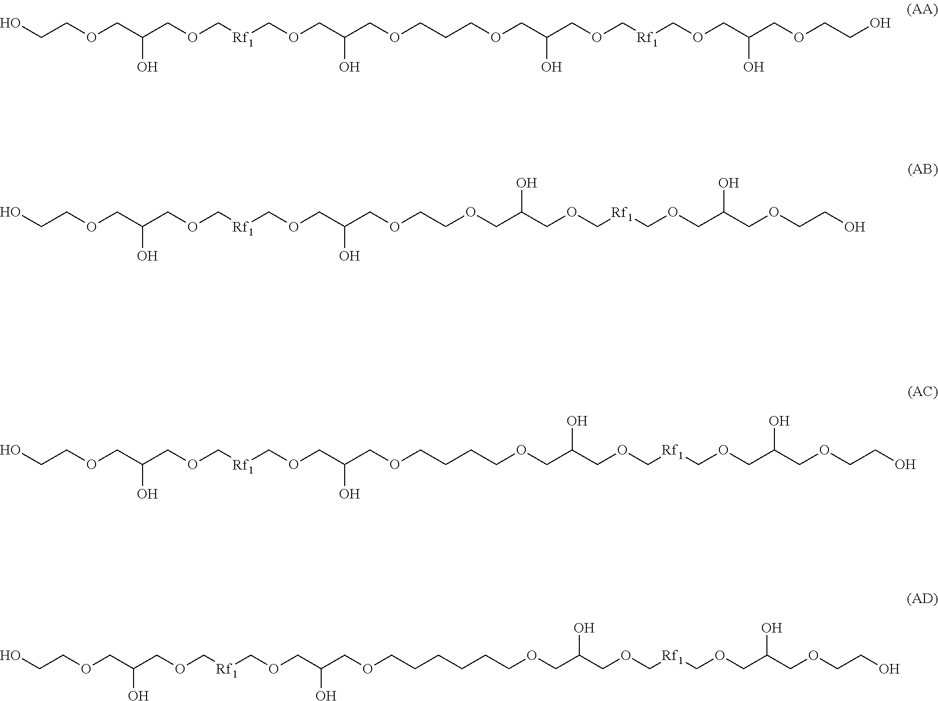

(In the two Rf₁'s in Formula (AA), h and i each indicate an average degree of polymerization, h represents 1 to 20, and i represents 0 to 20; and h and i in the two Rf₁'s may be the same as or different from each other)

in the two Rf₁'s in Formula (AB), h and i each indicate an average degree of polymerization, h represents 1 to 20, and i represents 0 to 20; and h and i in the two Rf₁'s

may be the same as or different from each other)
(n the two Rf₁'s in Formula (AC), h and i each indicate an average degree of polymerization, h represents 1 to 20, and i represents 0 to 20, where h and i in the two Rf₁'s may be the same as or different from each other)
(in the two Rf₁'s in Formula (AD), h and i each indicate an average degree of polymerization, h represents 1 to 20, and i represents 0 to 20; and h and i in the two Rf₁'s may be the same as or different from each other)

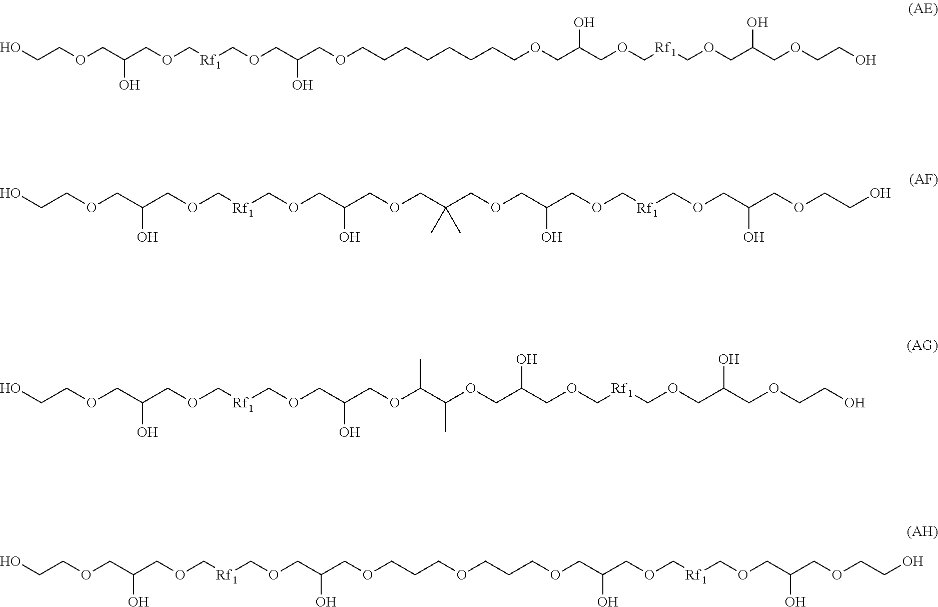

(in the two Rf₁'s in Formula (AE), h and i each indicate an average degree of polymerization, h represents 1 to 20, and i represents 0 to 20; and h and i in the two Rf₁'s may be the same as or different from each other)
(in the two Rf₁'s in Formula (AF), h and i each indicate an average degree of polymerization, h represents 1 to 20, and i represents 0 to 20; and h and i in the two Rf₁'s may be the same as or different from each other)
(in the two Rf₁'s in Formula (AG), h and i each indicate an average degree of polymerization, h represents 1 to 20, and i represents 0 to 20; and h and i in the two Rf₁'s may be the same as or different from each other)
(in the two Rf₁'s in Formula (AH), h and i each indicate an average degree of polymerization, h represents 1 to 20, and i represents 0 to 20; and h and i in the two Rf₁'s may be the same as or different from each other)

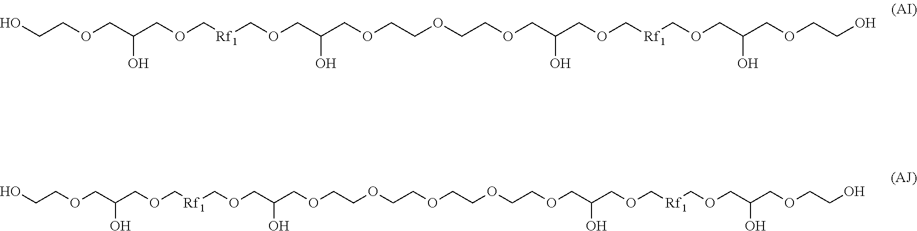

(in the two Rf₁'s in Formula (AI), h and i each indicate an average degree of polymerization, h represents 1 to 20, and i represents 0 to 20; and h and i in the two Rf₁'s may be the same as or different from each other)
(in the two Rf₁'s in Formula (AJ), h and i each indicate an average degree of polymerization, h represents 1 to 20, and i represents 0 to 20; and h and i in the two Rf₁'s may be the same as or different from each other)

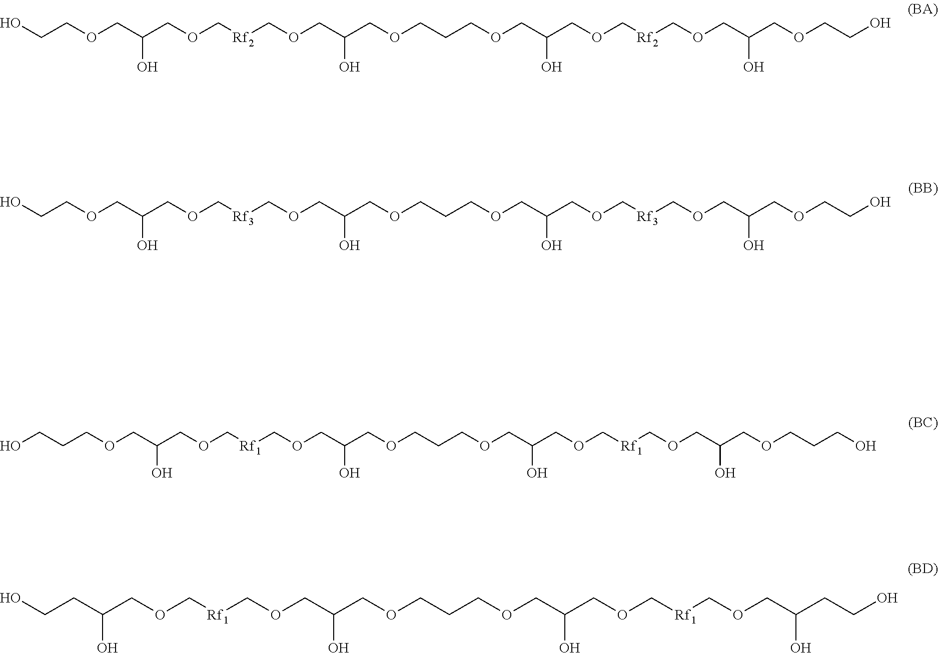

(in the two Rf₂'s in Formula (BA), j indicates an average degree of polymerization and represents 1 to 15; and j's of the two Rf₂'s may be the same as or different from each other)
(in the two Rf₃'s in Formula (BB), k indicates an average degree of polymerization and represents 1 to 10; and k's in the two Rf₃'s may be the same as or different from each other)
(n the two Rf₁'s in Formula (BC), h and i each indicate an average degree of polymerization, h represents 1 to 20, and i represents 0 to 20, where h and i in the two Rf₁'s may be the same as or different from each other)
(in the two Rf₁'s in Formula (BD), h and i each indicate an average degree of polymerization, h represents 1 to 20, and i represents 0 to 20; and h and i in the two Rf₁'s may be the same as or different from each other)

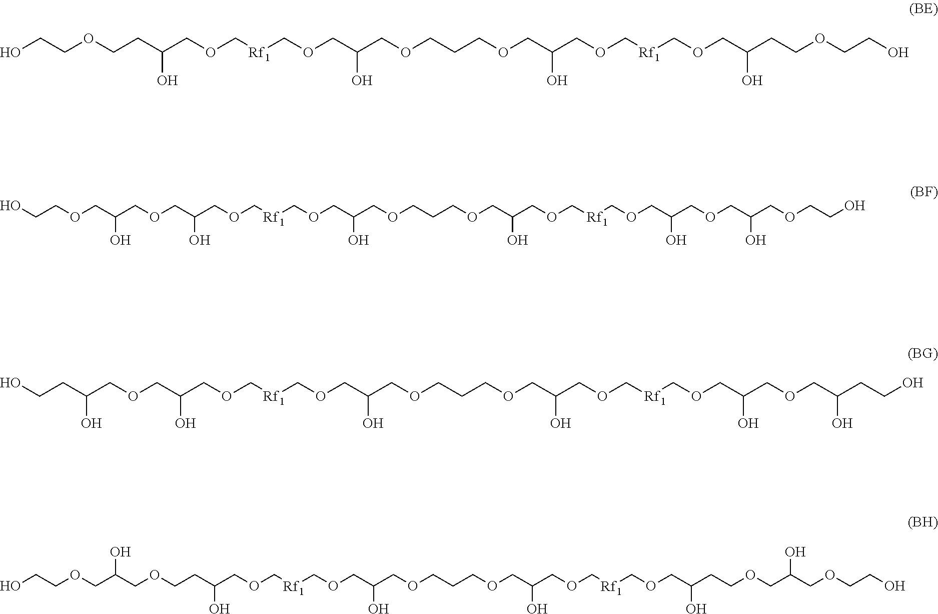

(n the two Rf₁'s in Formula (BE), h and i each indicate an average degree of polymerization, h represents 1 to 20, and i represents 0 to 20, where h and i in the two Rf₁'s may be the same as or different from each other)
in the two Rf₁'s in Formula (BF), h and i each indicate an average degree of polymerization, h represents 1 to 20, and i represents 0 to 20, and h and i in the two Rf₁'s may be the same as or different from each other)
(n the two Rf₁'s in Formula (BG), h and i each indicate an average degree of polymerization, h represents 1 to 20, and i represents 0 to 20, where h and i in the two Rf₁'s may be the same as or different from each other)
(in the two Rf₁'s in Formula (BH), h and i each indicate an average degree of polymerization, h represents 1 to 20, and i represents 0 to 20; and h and i in the two Rf₁'s may be the same as or different from each other)

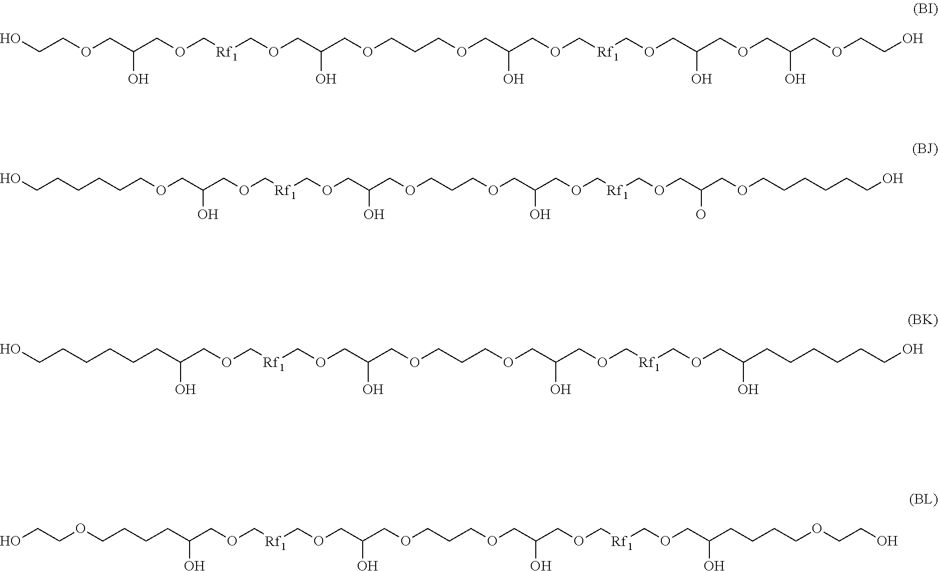

(in the two Rf₁'s in Formula (BI), h and i each indicate an average degree of polymerization, h represents 1 to 20, and i represents 0 to 20; and h and i in the two Rf₁'s may be the same as or different from each other)
(in the two Rf₁'s in Formula (BJ), h and i each indicate an average degree of polymerization, h represents 1 to 20, and i represents 0 to 20; and h and i in the two Rf₁'s may be the same as or different from each other)
(in the two Rf₁'s in Formula (BK), h and i each indicate an average degree of polymerization, h represents 1 to 20, and i represents 0 to 20; and h and i in the two Rf₁'s may be the same as or different from each other)
(in the two Rf₁'s in Formula (BL), h and i each indicate an average degree of polymerization, h represents 1 to 20, and i represents 0 to 20; and h and i in the two Rf₁'s may be the same as or different from each other)

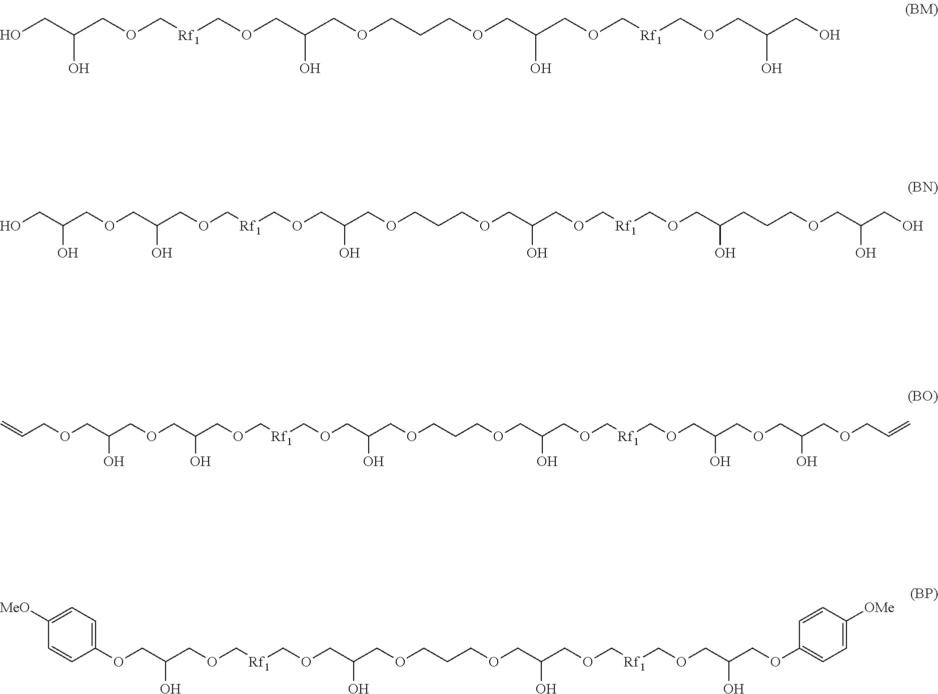

(in the two Rf₁'s in Formula (BM), h and i each indicate an average degree of polymerization, h represents 1 to 20, and i represents 0 to 20; and h and i in the two Rf₁'s may be the same as or different from each other)
(in the two Rf₁'s in Formula (BN), h and i each indicate an average degree of polymerization, h represents 1 to 20, and i represents 0 to 20; and h and i in the two Rf₁'s may be the same as or different from each other)
(in the two Rf₁'s in Formula (BO), h and i each indicate an average degree of polymerization, h represents 1 to 20, and i represents 0 to 20; and h and i in the two Rf₁'s may be the same as or different from each other)
(in the two Rf₁'s in Formula (BP), h and i each indicate an average degree of polymerization, h represents 1 to 20, and i represents 0 to 20; and h and i in the two Rf₁'s may be the same as or different from each other; and Me represents a methyl group)

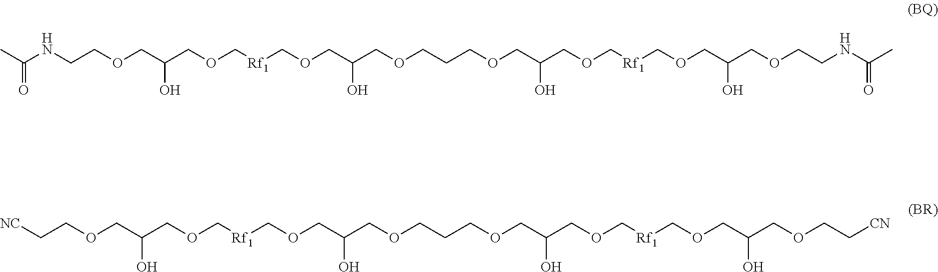

(in the two Rf₁'s in Formula (BQ), h and i each indicate an average degree of polymerization, h represents 1 to 20, and i represents 0 to 20; and h and i in the two Rf₁'s may be the same as or different from each other)
(in the two Rf₁'s in Formula (BR), h and i each indicate an average degree of polymerization, h represents 1 to 20, and i represents 0 to 20; and h and i in the two Rf₁'s may be the same as or different from each other)

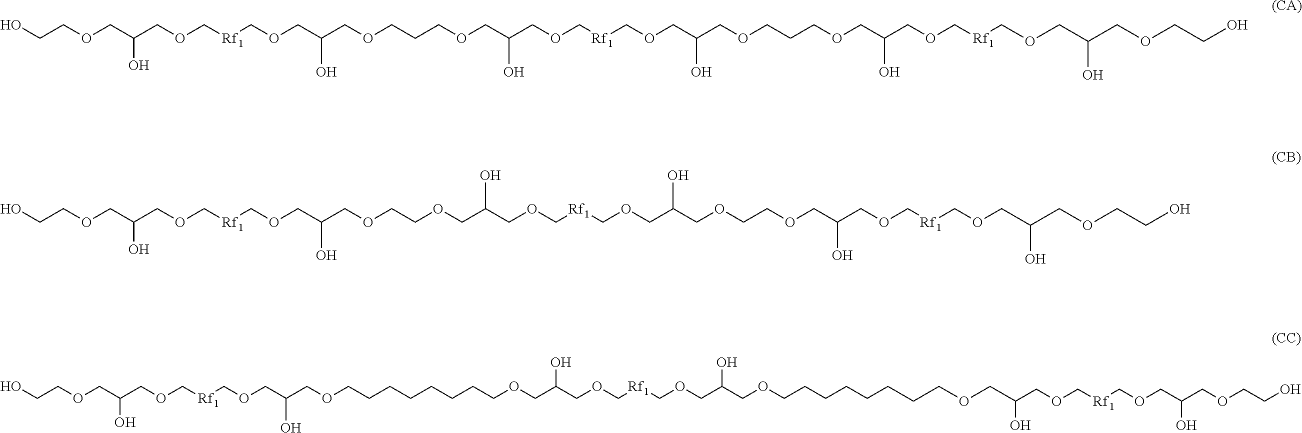

(in the three Rf₁'s in Formula (CA), ha and i each indicate an average degree of polymerization, h represents 1 to 20, and i represents 0 to 20; and h and i in the three Rf₁'s may be partially or entirely the same as or different from each other)
(in the three Rf₁'s in Formula (CB), h and i each indicate an average degree of polymerization, h represents 1 to 20, and i represents 0 to 20; and h and i in the three Rf₁'s may be partially or entirely the same as or different from each other)
(in the three Rf₁'s in Formula (CC), h and i each indicate an average degree of polymerization, h represents 1 to 20, and i represents 0 to 20; and h and i in the three Rfₗ's may be partially or entirely the same as or different from each other)

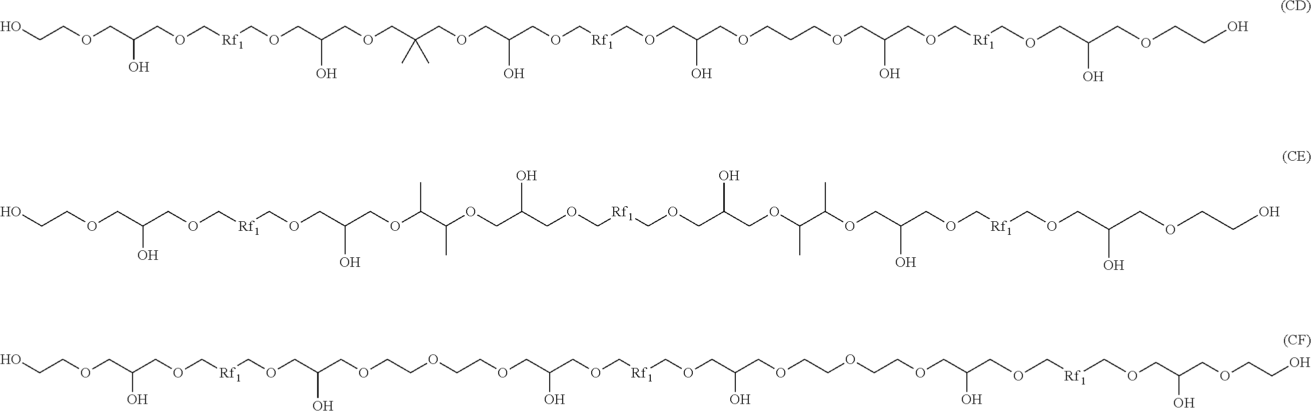

(in the three Rf₁'s in Formula (CD), h and i each indicate an average degree of polymerization. h represents 1 to 20. and i represents 0 to 20; and h and i in the three Rf₁'s may be partially or entirely the same as or different from each other)
(in the three Rf₁'s in Formula (CE), h and i each indicate an average degree of polymerization, h represents 1 to 20, and i represents 0 to 20; and h and i in the three Rf₁'s may be partially or entirely the same as or different from each other)
(in the three Rf₁'s in Formula (CF), h and i each indicate an average degree of polymerization, h represents 1 to 20, and i represents 0 to 20; and h and i in the three Rf₁'s may be partially or entirely the same as or different from each other)

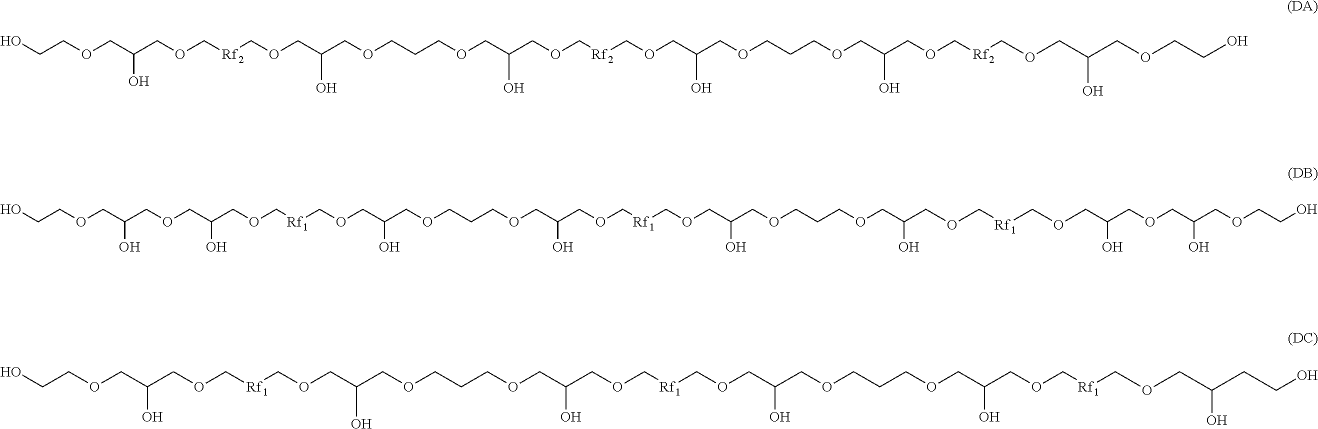

(in the three Rf₂'s in Formula (DA), j indicates an average degree of polymerization and represents 1 to 15; and j's in the three Rf₂'s may be partially or entirely the same as or different from each other)
(in the three Rf₁'s in Formula (DB), h and i each indicate an average degree of polymerization, h represents 1 to 20, and i represents 0 to 20; and h and i in the three Rf₁'s may be partially or entirely the same as or different from each other)
(in the three Rf₁'s in Formula (DC), h and i each indicate an average degree of polymerization, h represents 1 to 20, and i represents 0 to 20; and h and i n the three Rf₁'s may be partially or entirely the same as or different from each other)

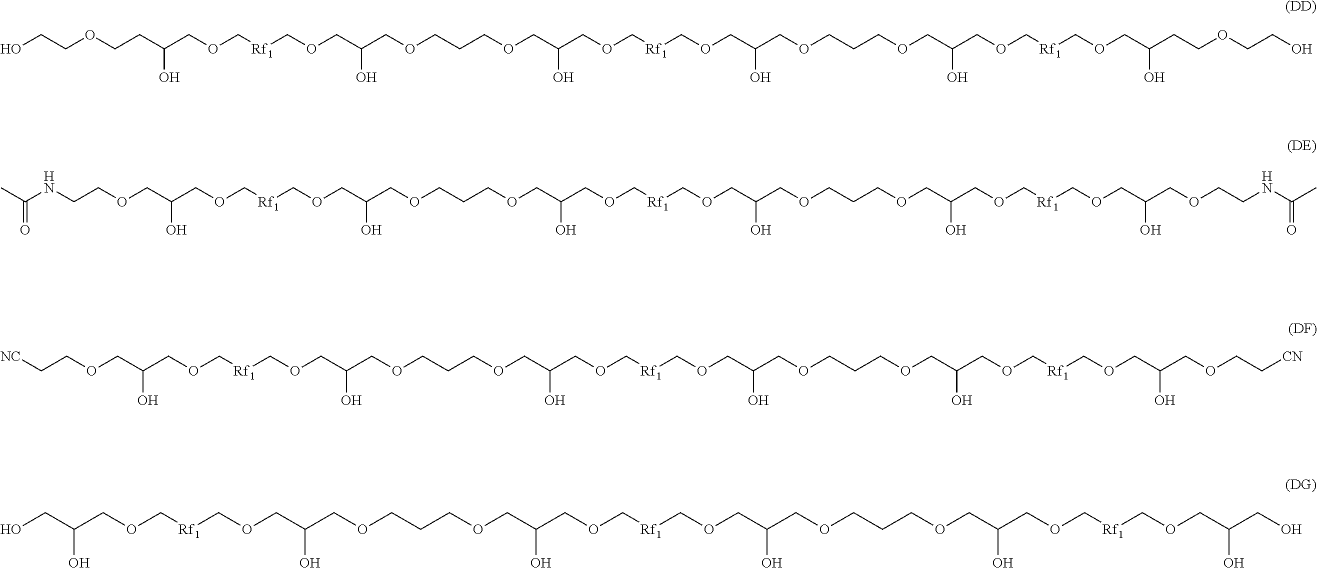

(In the three Rf₁'s in Formula (DD), h and i each indicate an average degree of polymerization, h represents 1 to 20, and i represents 0 to 20; and h and i in the three Rf₁'s may be partially or entirely the same as or different from each other.
(in the three Rf₁'s in Formula (DE), h and i each indicate an average degree of polymerization, h represents 1 to 20, and i represents 0 to 20; and h and i in the three Rf₁'s may be partially or entirely the same as or different from each other)
(in the three Rf₁'s in Formula (DF), h and i each indicate an average degree of polymerization, h represents 1 to 20, and i represents 0 to 20; and h and i in the three Rf₁'s may be partially or entirely the same as or different from each other)
(in the three Rf₁'s in Formula (DG), h and i each indicate an average degree of polymerization, h represents 1 to 20, and i represents 0 to 20; and h and i in the three Rf₁'s may be partially or entirely the same as or different from each other)

A number-average molecular weight (Mn) of the fluorine-containing ether compound of the present embodiment is preferably in a range of 500 to 10,000, and particularly preferably in a range of 1,000 to 5,000. In a case where the number-average molecular weight is 500 or more, the lubricating layer consisting of the lubricant including the fluorine-containing ether compound of the present embodiment has excellent heat resistance. The number-average molecular weight of the fluorine-containing ether compound is more preferably 1,000 or more. In addition, in a case where the number-average molecular weight is 10,000 or less, the viscosity of the fluorine-containing ether compound is appropriate, and a lubricating layer with a small film thickness can be easily formed by applying the lubricant including the fluorine-containing ether compound. The number-average molecular weight of the fluorine-containing ether compound is preferably 5,000 or less since a viscosity which is easy to handle is obtained in a case of being applied to the lubricant.

The number-average molecular weight (Mn) of the fluorine-containing ether compound is a value measured by ¹H-NMR measurement and ¹⁹F-NMR using AVANCE III 400 manufactured by Bruker BioSpin GmbH. Specifically, the number of repeating units of the PFPE chain is calculated from an integrated value measured by ¹⁹F-NMR to determine the number-average molecular weight. In the measurement of nuclear magnetic resonance (NMR), a sample is diluted with a solvent of hexafluorobenzene/d-acetone (4/1 v/v) and measured. A reference of the ¹⁹F-NMR chemical shift is a peak of hexafluorobenzene at −164.7 ppm and a reference of the ¹H-NMR chemical shift is a peak of acetone at 2.2 ppm.

The fluorine-containing ether compound of the present embodiment is preferably subjected to molecular weight fractionation by an appropriate method to have a polydispersity (ratio of a weight average molecular weight (Mw)/a number-average molecular weight (Mn)) of 1.3 or less.

In the present embodiment, a method for fractionating the molecular weight is not particularly limited, and for example, a molecular weight fractionation by a silica gel column chromatography method, a gel permeation chromatography (GPC) method, or the like, a molecular weight fractionation by a supercritical extraction method, or the like can be used.

### “Production Method”

A method for producing a fluorine-containing ether compound of the present embodiment is not particularly limited, and the fluorine-containing ether compound can be produced by a known production method in the related art. The fluorine-containing ether compound of the present embodiment can be produced, for example, by a production method shown below.

### [First Production Method (Case Where x Is 1)]

(Case Where R¹ and R⁴ Are Same as Each Other and Two R²'s Are Same as Each Other)

A fluorine-based compound in which a hydroxymethyl group (—CH₂OH) is arranged at each of both terminals of a perfluoropolyether chain corresponding to R² in Formula (1) is prepared.

Next, the hydroxy group of the hydroxymethyl group arranged at one of terminals of the fluorine-based compound and an epoxy compound having a group corresponding to R¹ (=a group corresponding to R⁴) in Formula (1) are reacted with each other (first reaction). Thus, an intermediate compound 1-1 having a group corresponding to R¹ (=a group corresponding to R⁴) at one of terminals of the perfluoropolyether chain corresponding to R² is obtained.

As the epoxy compound having a group corresponding to R¹ (=a group corresponding to R⁴) in Formula (1), for example, compounds represented by Formulae (5-1) to (5-16) can be used. THP in Formulae (5-1) to (5-13) represents a tetrahydropyranyl group. Me in Formula (5-14) represents a methyl group.

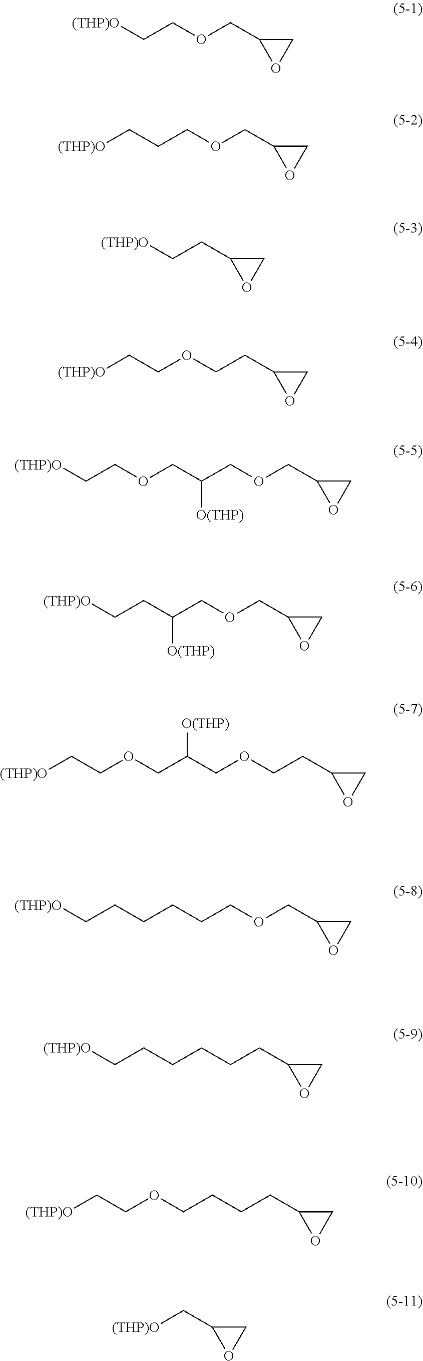

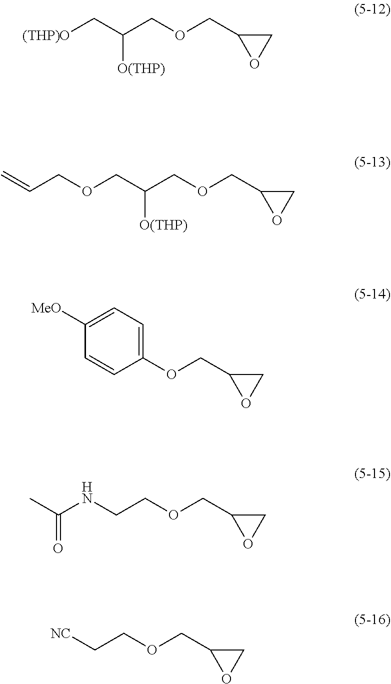

The epoxy compound having a group corresponding to R¹ (=a group corresponding to R⁴) in Formula (1) can be produced by a method shown below. That is, the compound can be produced by preparing a diol having a structure corresponding to a part of the terminal group represented by R¹ or R⁴ in Formula (1), protecting one of hydroxy groups thereof by a known method, and then reacting the diol with a halogen compound such as a bromine compound having an epoxy group and a chlorine compound. For example, in a case of producing a compound represented by Formula (5-2), the compound can be produced using a method in which one of hydroxy groups of glycol having a corresponding structure is protected with dihydropyran (DHP) and epibromohydrin is reacted therewith, as shown in Formula (6-1). THP in Formula (6-1) represents a tetrahydropyranyl group.

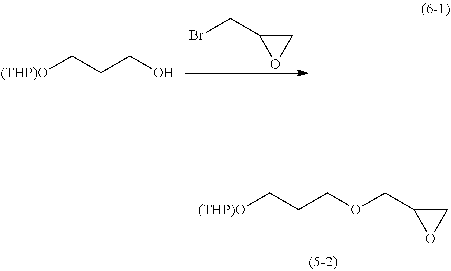

The epoxy compound may be produced by a method shown below. That is, the hydroxy group of 2-(2-bromoethoxy)tetrahydro-2H-pyran and an alcohol having an alkenyl group corresponding to a part of the terminal group represented by R¹ or R⁴ in Formula (1) are reacted with each other. Thereafter, a method in which the obtained compound is oxidized by reacting m-chloroperbenzoic acid (mCPBA) therewith can be used to produce the epoxy compound. For example, a compound represented by Formula (5-4) can be produced by a method in which 2-(2-bromoethoxy)tetrahydro-2H-pyran and 3-buten-1-ol are reacted with each other, and then the product is oxidized by reacting m-chloroperbenzoic acid therewith, as shown in Formula (6-2). THP in Formula (6-2) represents a tetrahydropyranyl group.

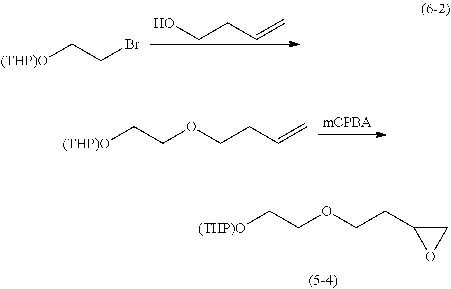

The epoxy compound may be produced by a method shown below. That is, an epoxy compound having a structure corresponding to a part of the terminal group represented by R¹ or R⁴ in Formula (1) and having a hydroxy group protected with a protective group at one end is produced by a known method. The obtained compound is subjected to an addition reaction with an alcohol having an alkenyl group corresponding to a part of the terminal group represented by R¹ or R⁴ in Formula (1). Thereafter, a method in which the obtained compound is oxidized by reacting m-chloroperbenzoic acid (mCPBA) therewith can be used to produce the epoxy compound. Before the compound obtained by the addition reaction is oxidized with m-chloroperbenzoic acid (mCPBA), a hydroxy group generated by the addition reaction may be protected with a known method. For example, a compound represented by Formula (5-5) can be produced by a method in which the epoxy compound represented by Formula (5-1) and an allyl alcohol are subjected to an addition reaction, and then protected using dihydropyran (DHP), and the product is oxidized with m-chloroperbenzoic acid (mCPBA), as shown in Formula (6-3). THP in Formula (6-3) represents a tetrahydropyranyl group.

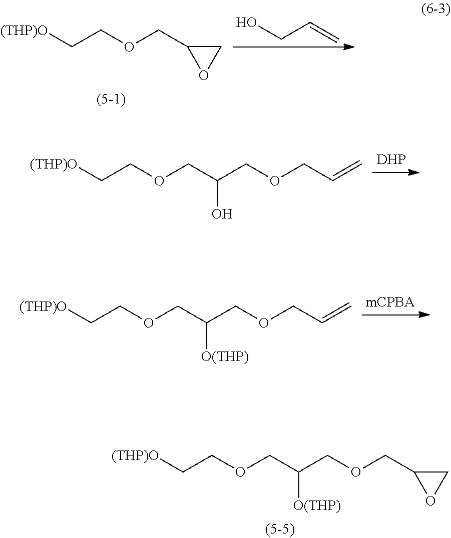

Thereafter, the hydroxy group of the hydroxymethyl group arranged at one of terminals of the intermediate compound 1-1 generated in the above-described first reaction and a compound having two epoxy groups corresponding to R³ in Formula (1) are reacted with each other (second reaction).

As the compound having two epoxy groups corresponding to R³ in Formula (1), for example, compounds represented by Formulae (7-1) to (7-10) can be used.

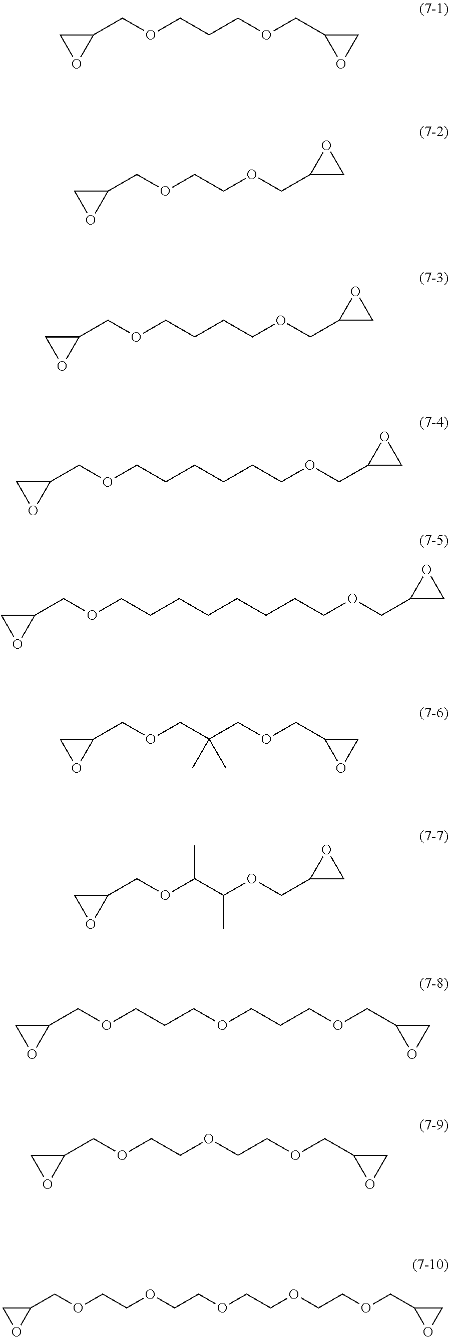

The compound having two epoxy groups corresponding to R³ in Formula (1) can be produced by a method shown below. That is, a diol having a structure corresponding to Y in Formula (2) of the divalent linking group represented by R³ and epibromohydrin in a molar amount twice that of the diol are reacted with each other to produce the compound of Formula (2). For example, the compound represented by Formula (7-1) can be produced by a method in which 1,3-propanediol is reacted with epibromohydrin in a molar amount twice that 1,3-propanediol, as shown in Formula (8-1).

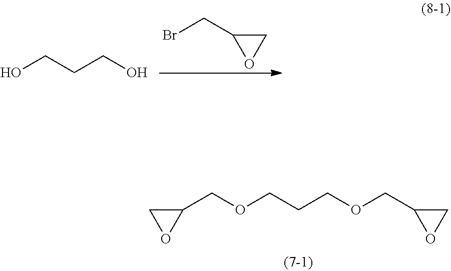 [SMILES: OCCCO ; BrCC1CO1 ; C(COCC1CO1)COCC1CO1]

The compound having two epoxy groups corresponding to R³ in Formula (1) may be produced by a method of oxidizing a compound having two alkenyl groups by reacting m-chloroperbenzoic acid (mCPBA) therewith, and a commercially available product may be used.

After the steps above, a deprotection reaction can be performed by a known method to produce a compound in which x in Formula (1) is 1, R¹ and R⁴ are the same as each other, and two R²'s are the same as each other.

### [Second Production Method (Case Where x Is 1)]

(Case Where R¹ and R⁴ Are Different from Each Other and/or Two R²'s Are Different from Each Other)

First, the hydroxy group at one of terminals of a fluorine-based compound in which hydroxymethyl groups are each arranged at both terminals of a perfluoropolyether chain corresponding to R² on the R¹ side is reacted with an epoxy compound having a group corresponding to R¹ to obtain an intermediate compound 1 a (first reaction).

Next, the hydroxy group at one of terminals of a fluorine-based compound in which hydroxymethyl groups are each arranged at both terminals of a perfluoropolyether chain corresponding to R² on the R⁴ side is reacted with an epoxy compound having a group corresponding to R⁴ to obtain an intermediate compound 1b (second reaction).

Next, the hydroxy group at one of terminals of the intermediate compound 1a and a compound having an epoxy group and an alkenyl group corresponding to R³ in Formula (1) are reacted with each other, and then a double bond contained in the generated compound is oxidized to obtain an intermediate compound 1-2 (third reaction). As the compound having an epoxy group and an alkenyl group corresponding to R³ in Formula (1), for example, compounds represented by Formulae (9-1) to (9-10) which will be described later can be used.

Next, the hydroxy group at one of terminals of the intermediate compound 1b and the epoxy group of the intermediate compound 1-2 obtained in the third reaction are reacted with each other (fourth reaction).

After the step above, a deprotection reaction can be performed using a known method to produce a compound in which x in Formula (1) is 1, R¹ and R⁴ are different from each other, and/or two R²'s are different.

[Third Production Method (Case where x is 2)]
(Case Where R¹ and R⁴ Are Same as Each Other, Two R³'s Are Same as Each Other, and R² on R¹ Side and R² on R⁴ Side Are Same as Each Other)

First, an intermediate compound 1-1 having a group corresponding to R¹ (=a group corresponding to R⁴) at one of terminals of the perfluoropolyether chain corresponding to R² on the R¹ side and the R⁴ side is obtained in the same manner as in the first production method (first reaction).

Next, a fluorine-based compound in which a hydroxymethyl group (—CH₂OH) is arranged at each of both terminals of a perfluoropolyether chain corresponding to R² at the center of the molecule in Formula (1) is prepared. Next, the hydroxy group of the hydroxymethyl group arranged at both terminals of the fluorine-based compound and a compound having an epoxy group and an alkenyl group corresponding to R³ in Formula (1) are reacted with each other to obtain an intermediate compound 2-1 (second reaction).

As the compound having an epoxy group and an alkenyl group corresponding to R³ in Formula (1), for example, the compounds represented by Formulae (9-1) to (9-10) can be used.

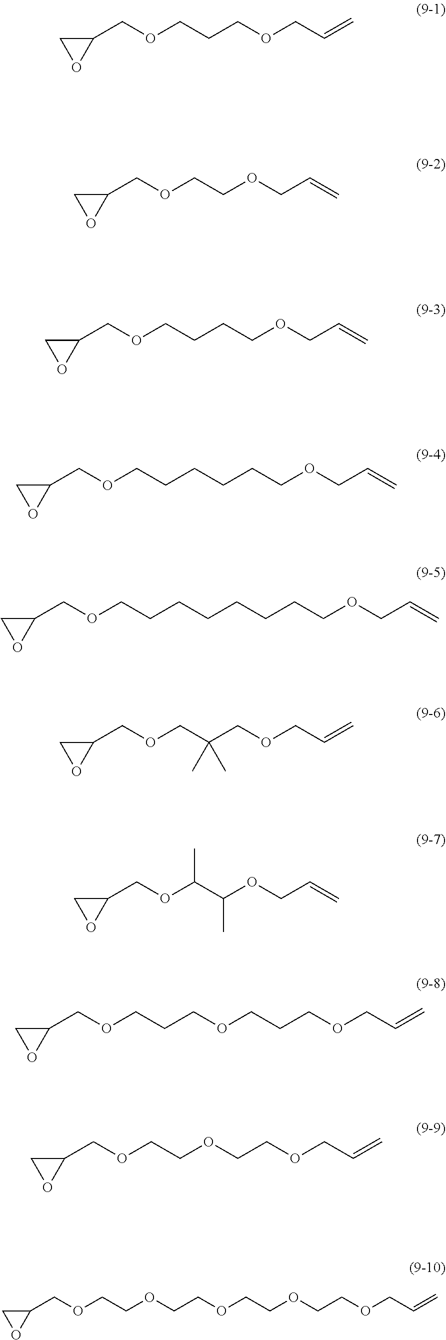

The compound having an epoxy group and an alkenyl group corresponding to R³ in Formula (1) can be produced by a method shown below. That is, a diol having a structure corresponding to Y in Formula (2) of the divalent linking group represented by R³, allyl bromide, and epibromohydrin are reacted in this order to produce the compound of Formula (2). For example, the compound represented by Formula (9-1) can be produced by a method in which 1,3-prop anediol, allyl bromide, and epibromohydrin are reacted in this order, as shown in Formula (10-1).

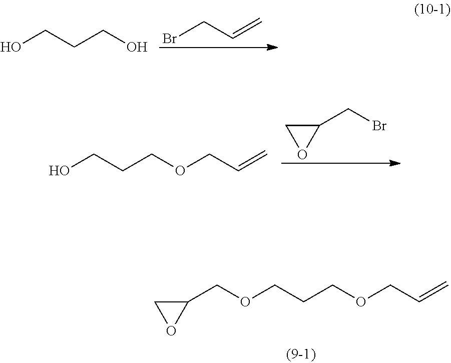

The compound having an epoxy group and an alkenyl group, corresponding to R³ in Formula (1), may be produced by a method in which a compound having two alkenyl groups is reacted with m-chloroperbenzoic acid (mCPBA) to oxidize one alkenyl group, and a commercially available product may be used.

Next, the intermediate compound 2-1 generated in the second reaction is oxidized by reacting m-chloroperbenzoic acid (mCPBA) therewith (third reaction). Thus, an intermediate compound 3-1 having epoxy groups corresponding to two R³'s in Formula (1) at both terminals of the perfluoropolyether chain corresponding to R² at the center of the molecule in Formula (1) is obtained. The third reaction may be carried out after appropriately protecting the hydroxy group contained in the intermediate compound 2-1 by a known method.

Thereafter, the hydroxy group of the hydroxymethyl group arranged at one of terminals of the intermediate compound 1-1 and epoxy groups arranged at both terminals of the intermediate compound 3-1 are reacted with each other (fourth reaction).

By performing a deprotection reaction after the step above, a compound in which x in Formula (1) is 2, R¹ and R⁴ are the same as each other, two R³'s are the same as each other, and R² on the R¹ side and R² on the R⁴ side are the same as each other can be produced.

### [Fourth Production Method (Case Where x Is 2)]

(Case Where R³ on R Side and R³ on R⁴ Side Are Same as Each Other, R¹ and R⁴ Are Different From Each Other, and/or R² on R¹ Side and R² on R⁴ Side Are Different From Each Other)

In the first reaction of the third production method, the intermediate compound 1a and the intermediate compound 1b in the second production method are obtained instead of the intermediate compound 1-1. Next, an intermediate compound 3-1 is obtained in the same manner as in the second reaction and the third reaction of the third production method. Then, the intermediate compound 1a and the intermediate compound 1b are each sequentially reacted with the epoxy groups arranged at both terminals of the intermediate compound 3-1.

By performing a deprotection reaction after the step above, a compound in which x in Formula (1) is 2, R³ on the R¹ side and R³ on the R⁴ side are the same as each other, R¹ and R⁴ are different from each other, and/or R² on the R¹ side and R² on the R⁴ side are different from each other can be produced.

### [Fifth Production Method (Case Where x Is 2)]

(Case Where R³ on R¹ Side and R³ on R⁴ Side Are Different From Each Other, R¹ and R⁴ Are Same as Each Other, and R² on R¹ Side and R² on R⁴ Side Are Same)

An intermediate compound 2-2 is obtained by reacting a compound having an epoxy group and an alkenyl group, corresponding to R³ on the R¹ side in Formula (1), and a compound having an epoxy group and an alkenyl group, corresponding to R³ on the R⁴ side in Formula (1), instead of the compound having an epoxy group and an alkenyl group corresponding to R³ in Formula (1), sequentially with the fluorine-based compound in the second reaction of the third production method. Then, the third reaction and the fourth reaction are carried out in the same manner as in the third production method, except that the intermediate compound 2-2 is used instead of the intermediate compound 2-1.

By performing a deprotection reaction after the step above, a compound in which x in Formula (1) is 2, R³ on the R¹ side and R³ on the R⁴ side are different from each other, R¹ and R⁴ are the same as each other, and R² on the R¹ side and R² on the R⁴ side are the same as each other can be produced.

In the third fifth production methods for producing the compound in which x is 2, the perfluoropolyether chain corresponding to R² at the center of the molecule may be the same as or different from the other R².

### [Lubricant for Magnetic Recording Medium]

The lubricant for a magnetic recording medium of the present embodiment includes the fluorine-containing ether compound represented by Formula (1).

In the lubricant of the present embodiment, known materials used as a material for the lubricant can be used in mixture as necessary as long as the characteristics are not impaired by incorporating the fluorine-containing ether compound represented by Formula (1).

Specific examples of the known materials include FOMBLIN (registered trademark) ZDIAC, FOMBLIN ZDEAL, and FOMBLIN AM-2001 (all of which are manufactured by Solvay Solexis.), and Moresco A20H (manufactured by Moresco).

The known material used in mixture with the lubricant of the present embodiment preferably has a number-average molecular weight of 1,000 to 10,000.

In a case where the lubricant of the present embodiment includes a material other than the fluorine-containing ether compound represented by Formula (1), a content of the fluorine-containing ether compound represented by Formula (1) in the lubricant of the present embodiment is preferably 70% by mass or more, more preferably 90% by mass or more, and still more preferably 95% by mass or more.

Since the lubricant of the present embodiment includes the fluorine-containing ether compound represented by Formula (1), it is possible to form a lubricating layer having excellent chemical substance resistance and a high spin-off suppressing effect.

### [Magnetic Recording Medium]

The magnetic recording medium of the present embodiment is formed by sequentially providing at least a magnetic layer, a protective layer, and a lubricating layer on a substrate.

In the magnetic recording medium of the present embodiment, one or two or more underlayers can be provided between the substrate and the magnetic layer, as necessary. In addition, at least one of an adhesion layer and a soft magnetic layer can be provided between the underlayer and the substrate.

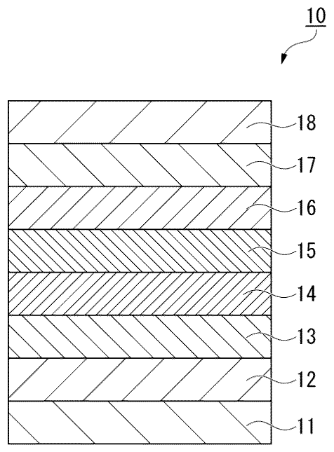

FIG. 1 is a schematic cross-sectional view showing an embodiment of the magnetic recording medium of the present invention.

The magnetic recording medium 10 of the present embodiment has a structure in which an adhesion layer 12, a soft magnetic layer 13, a first underlayer 14, a second underlayer 15, a magnetic layer 16, a protective layer 17, and a lubricating layer 18 are sequentially provided on a substrate 11.

### “Substrate”

As the substrate 11, for example, a non-magnetic substrate on which a film made of NiP or an NiP alloy is formed on a base made of a metal or an alloy material such as Al or an Al alloy can be used.

In addition, as the substrate 11, a non-magnetic substrate made of a non-metal material such as glass, ceramics, silicon, silicon carbide, carbon, or a resin may be used, or a non-magnetic substrate in which a film of NiP or an NiP alloy is formed on a base made of this non-metal material may be used.

“Adhesion Layer” The adhesion layer 12 prevents the progress of corrosion of the substrate 11, which occurs in a case where the substrate 11 and the soft magnetic layer 13 provided on the adhesion layer 12 are arranged in contact with each other.

A material for the adhesion layer 12 can be appropriately selected from, for example, Cr, a Cr alloy, Ti, a Ti alloy, CrTi, NiAl, and an AIRu alloy. The adhesion layer 12 can be formed by, for example, a sputtering method.

### “Soft Magnetic Layer”

The soft magnetic layer 13 preferably has a structure in which a first soft magnetic film, an intermediate layer consisting of an Ru film, and a second soft magnetic film are sequentially laminated. That is, the soft magnetic layer 13 preferably has a structure in which the intermediate layer made of an Ru film is interposed between two soft magnetic film layers to make the soft magnetic films above and below the intermediate layer be antiferromagnetically coupled (AFC).

Examples of materials for the first soft magnetic film and the second soft magnetic film include a CoZrTa alloy and a CoFe alloy.

Any of Zr, Ta, and Nb is preferably added to the CoFe alloy which is used in the first soft magnetic film and the second soft magnetic film. This accelerates the amorphization of the first soft magnetic film and the second soft magnetic film. As a result, it is possible to improve the alignment of the first underlayer (seed layer) and to decrease the floating amount of a magnetic head.

The soft magnetic layer 13 can be formed by, for example, a sputtering method.

### “First Underlayer”

The first underlayer 14 is a layer that controls the alignment and the crystal size of the second underlayer 15 and the magnetic layer 16 provided thereon.

Examples of the first underlayer 14 include a Cr layer, a Ta layer, an Ru layer, a CrMo alloy layer, a CoW alloy layer, a CrW alloy layer, a CrV alloy layer, and a CrTi alloy layer.

The first underlayer 14 can be formed by, for example, a sputtering method.

### “Second Underlayer”

The second underlayer 15 is a layer that controls the alignment of the magnetic layer 16 to be good. The second underlayer 15 is preferably a layer consisting of Ru or an Ru alloy.

The second underlayer 15 may be a single layer or may be composed of a plurality of layers. In a case where the second underlayer 15 consists of a plurality of layers, all the layers may be composed of the same material, or at least one layer may be composed of a different material.

The second underlayer 15 can be formed by, for example, a sputtering method.

### “Magnetic Layer”

The magnetic layer 16 consists of a magnetic film in which a magnetization axis is oriented in a direction perpendicular or horizontal to the substrate surface. The magnetic layer 16 is a layer including Co and Pt. The magnetic layer 16 may be a layer including an oxide, Cr, B, Cu, Ta, Zr, or the like in order to improve the SNR characteristics.

Examples of the oxide contained in the magnetic layer 16 include SiO₂, SiO, Cr₂O₃, CoO, Ta₂O₃, and TiO₂.

The magnetic layer 16 may be composed of one layer, or may be composed of a plurality of magnetic layers made of materials with different compositions. For example, in a case where the magnetic layer 16 consists of three layers of a first magnetic layer, a second magnetic layer, and a third magnetic layer which are laminated in this order from the bottom, the first magnetic layer preferably has a granular structure made of a material containing Co, Cr, and Pt and further containing an oxide. As the oxide contained in the first magnetic layer, for example, an oxide of Cr, Si, Ta, Al, Ti, Mg, Co, or the like is preferably used. Among these, TiO₂, Cr₂O₃, SiO₂, or the like can be particularly suitably used. In addition, it is preferable that the first magnetic layer consists of a composite oxide obtained by adding two or more kinds of oxides. Among these, Cr₂O₃—SiO₂, Cr₂O₃—TiO₂, SiO₂—TiO₂, or the like can be particularly suitably used.

The first magnetic layer can include one or more kinds of elements selected from B, Ta, Mo, Cu, Nd, W, Nb, Sm, Tb, Ru, and Re, in addition to Co, Cr, Pt, and oxides.

The same material as that for the first magnetic layer can be used for the second magnetic layer. The second magnetic layer preferably has a granular structure.

The third magnetic layer preferably has a non-granular structure consisting of a material including Co, Cr, and Pt and not including an oxide. The third magnetic layer can include one or more elements selected from B, Ta, Mo, Cu, Nd, W, Nb, Sm, Tb, Ru, Re, and Mn, in addition to Co, Cr, and Pt.

In a case where the magnetic layer 16 is formed of a plurality of magnetic layers, it is preferable to provide a non-magnetic layer between the adjacent magnetic layers. In a case where the magnetic layer 16 consists of three layers of a first magnetic layer, a second magnetic layer, and a third magnetic layer, it is preferable to provide a non-magnetic layer between the first magnetic layer and the second magnetic layer and between the second magnetic layer and the third magnetic layer.

As the non-magnetic layer provided between the adjacent magnetic layers of the magnetic layer 16, for example, Ru, an Ru alloy, a CoCr alloy, or a CoCrX1 alloy (X1 represents one or two or more kinds of elements selected from Pt, Ta, Zr, Re, Ru, Cu, Nb, Ni, Mn, Ge, Si, O, N, W, Mo, Ti, V, and B) can be suitably used.

It is preferable that an alloy material including an oxide, a metal nitride, or a metal carbide is used in the non-magnetic layer provided between the adjacent magnetic layers of the magnetic layer 16. Specifically, as the oxide, for example, SiO₂, Al₂O₃, Ta₂O₅, Cr₂O₃, MgO, Y₂O₃, or TiO₂, can be used. As the metal nitride, for example, AlN, Si₃N₄, TaN, or CrN can be used. As the metal carbide, for example, TaC, BC, or SiCe can be used.

The non-magnetic layer can be formed by, for example, a sputtering method.

The magnetic layer 16 is preferably a magnetic layer for perpendicular magnetic recording in which easy magnetization axis is directed to a direction perpendicular to the substrate surface in order to realize a higher recording density. The magnetic layer 16 may be a magnetic layer for in-plane magnetic recording.

The magnetic layer 16 may be formed by any of known methods in the related art, such as a deposition method, an ion beam sputtering method, and a magnetron sputtering method. The magnetic layer 16 is usually formed by a sputtering method.

### “Protective Layer”

The protective layer 17 protects the magnetic layer 16. The protective layer 17 may be composed of one layer or may be composed of a plurality of layers. As the protective layer 17, a carbon-based protective layer can be preferably used, and in particular, an amorphous carbon protective layer is preferable. In a case where the protective layer 17 is a carbon-based protective layer, the interaction with the polar group (in particular, the hydroxy group) included in the fluorine-containing ether compound in the lubricating layer 18 is further enhanced, which is thus preferable.

An adhesive force between the carbon-based protective layer and the lubricating layer 18 can be controlled by forming the carbon-based protective layer with hydrogenated carbon and/or nitrogenated carbon and adjusting the hydrogen content and/or the nitrogen content in the carbon-based protective layer. The hydrogen content in the carbon-based protective layer, as measured by hydrogen forward scattering (HFS), is preferably 3 atomic % to 20 atomic %. In addition, the nitrogen content in the carbon-based protective layer, measured by an X-ray photoelectron spectroscopy (XPS), is preferably 4 atomic % to 15 atomic %.

Hydrogen and/or nitrogen included in the carbon-based protective layer does not need to be evenly contained in the entire carbon-based protective layer. For example, the carbon-based protective layer is suitably formed as a composition gradient layer in which nitrogen is contained in the protective layer 17 on the lubricating layer 18 side and hydrogen is contained in the protective layer 17 on the magnetic layer 16 side. In this case, the adhesive force between the magnetic layer 16 and the carbon-based protective layer and between the lubricating layer 18 and the carbon-based protective layer is further improved.

A film thickness of the protective layer 17 is preferably 1 nm to 7 nm. In a case where the film thickness of the protective layer 17 is 1 nm or more, sufficient performance of the protective layer 17 can be obtained. The film thickness of the protective layer 17, which is 7 nm or less, is preferable from the viewpoint that the thickness of the protective layer 17 is reduced.

As a method for forming the protective layer 17, a sputtering method using a carbon-containing target material, a chemical vapor deposition (CVD) method using a hydrocarbon raw material such as ethylene and toluene, an ion beam deposition (IBD) method, or the like can be used.

In a case where the carbon-based protective layer is formed as the protective layer 17, the carbon-based protective layer can be formed, for example, by a DC magnetron sputtering method. In particular, in a case where the carbon-based protective layer is formed as the protective layer 17, it is preferable to form an amorphous carbon protective layer by a plasma CVD method. The amorphous carbon protective layer formed by the plasma CVD method has an even surface and a small roughness.

### “Lubricating Layer”

The lubricating layer 18 prevents contamination of the magnetic recording medium 10. In addition, the lubricating layer 18 reduces a frictional force of a magnetic head of a magnetic recording and reproducing device which slides on the magnetic recording medium 10, thereby improving the durability of the magnetic recording medium 10.

As shown in FIG. 1, the lubricating layer 18 is formed on and in contact with the protective layer 17. The lubricating layer 18 is formed by applying the lubricant for a magnetic recording medium according to the above-described embodiment onto the protective layer 17. Therefore, the lubricating layer 18 includes the above-described fluorine-containing ether compound.

In a case where the protective layer 17 arranged below the lubricating layer 18 is a carbon-based protective layer, the lubricating layer 18 is particularly bonded to the protective layer 17 with a high bonding force. As a result, it is easy to obtain the magnetic recording medium 10 in which the surface of the protective layer 17 is coated at a high coating rate even in a case where the thickness of the lubricating layer 18 is small, and it is possible to effectively prevent the contamination of the surface of the magnetic recording medium 10.

An average film thickness of the lubricating layer 18 is preferably 0.5 nm (5 Å) to 2.0 nm (20 Å), and more preferably 0.5 nm (5 Å) to 1.2 nm (12 Å). In a case where the average film thickness of the lubricating layer 18 is 0.5 nm or more, the lubricating layer 18 is formed with an even film thickness without forming an island shape or a mesh shape. Therefore, the surface of the protective layer 17 can be coated with the lubricating layer 18 at a high coating rate. In addition, in a case where the average film thickness of the lubricating layer 18 is 2.0 nm or less, it is possible to sufficiently reduce the thickness of the lubricating layer 18, and to sufficiently decrease the floating amount of a magnetic head.

### “Method for Forming Lubricating Layer”

Examples of a method for forming the lubricating layer 18 include a method in which a magnetic recording medium during production in which respective layers up to the protective layer 17 are formed on the substrate 11 is prepared, and a lubricating layer forming solution is applied onto the protective layer 17 and dried.

The lubricating layer forming solution can be obtained by dispersing and dissolving the lubricant for a magnetic recording medium of the above-described embodiment in a solvent as necessary, and setting the viscosity and concentration to be suitable for application methods.

Examples of the solvent used for the lubricating layer forming solution include a fluorine-based solvent such as VERTREL (registered trademark) XF (product name, manufactured by Mitsui DuPont Fluorochemicals Co., Ltd.).

A method for applying the lubricating layer forming solution is not particularly limited, and examples thereof include a spin coating method, a spraying method, a paper coating method, and a dipping method.

In a case where the dipping method is used, for example, the following method can be used. First, the substrate 11 on which each of layers up to the protective layer 17 is formed is immersed in the lubricating layer forming solution which has been put into an immersion vessel of a dip coater. Next, the substrate 11 is lifted from the immersion vessel at a predetermined speed. Thus, the lubricating layer forming solution is applied onto the surface of the protective layer 17 of the substrate 11.

By using the dipping method, the lubricating layer forming solution can be evenly applied onto the surface of the protective layer 17 and the lubricating layer 18 with an even film thickness can be formed on the protective layer 17.

In the present embodiment, it is preferable to perform a thermal treatment on the substrate 11 on which the lubricating layer 18 has been formed. By performing the thermal treatment, the adhesion between the lubricating layer 18 and the protective layer 17 is improved, and the adhesive force between the lubricating layer 18 and the protective layer 17 is improved.

The thermal treatment temperature is preferably 100° C. to 180° C., and more preferably 100° C. to 160° C. In a case where the thermal treatment temperature is 100° C. or higher, an effect of improving the adhesion between the lubricating layer 18 and the protective layer 17 can be sufficiently obtained. In addition, by setting the thermal treatment temperature to 180° C. or lower, it is possible to prevent thermal decomposition of the lubricating layer 18 due to the thermal treatment. The thermal treatment time can be appropriately adjusted depending on the thermal treatment temperature, and is preferably 10 minutes to 120 minutes.

In the present embodiment, in order to further improve the adhesion of the lubricating layer 18 to the protective layer 17, a treatment of irradiating the lubricating layer 18 with ultraviolet rays (UV) before or after the thermal treatment may be performed.

The magnetic recording medium 10 of the present embodiment is formed by sequentially providing at least the magnetic layer 16, the protective layer 17, and the lubricating layer 18 on the substrate 11. In the magnetic recording medium 10 of the present embodiment, the lubricating layer 18 including the above-described fluorine-containing ether compound is formed on and in contact with the protective layer 17. The lubricating layer 18 has good chemical substance resistance and a high spin-off suppressing effect even in a case where the film thickness is small. Therefore, the magnetic recording medium 10 of the present embodiment has excellent reliability, in particular, silicon contamination suppression and durability. Thus, the magnetic recording medium 10 of the present embodiment can contribute to a reduction in magnetic spacing, and can be stably operated for a long period of time even in a severe environment due to the diversification of applications. Therefore, the magnetic recording medium 10 of the present embodiment is particularly suitable as a magnetic disk mounted in a magnetic disk device of a load unload (LUL) system.

### EXAMPLES

Hereinafter, the present invention will be described in more detail with reference to Examples and Comparative Examples. The present invention is not limited to only Examples described below.

### Example 1

The compound represented by Formula (AA) was obtained by a method shown below.

### (First Reaction)

20 g of a compound (number-average molecular weight: 1,000, molecular weight distribution: 1.1) represented by HOCH₂CF₂O(CF₂CF₂O)ₕ(CF₂O)ₕCF₂CH₂OH (in the formula, h indicating an average degree of polymerization is 4.5 and i indicating an average degree of polymerization is 4.5), 4.12 g of the compound represented by Formula (5-1), and 20 mL of t-butanol were charged into a 100 mL eggplant flask in a nitrogen gas atmosphere, and stirred at room temperature until a homogenous mixture was obtained. 1.12 g of potassium tert-butoxide was added to the mixture and stirred at 70° C. for 16 hours to undergo a reaction.

The compound represented by Formula (5-1) was synthesized by protecting a hydroxy group of ethylene glycol monoallyl ether using dihydropyran, and then oxidizing the product with m-chloroperbenzoic acid.

The reaction product obtained after the reaction was cooled to 25° C., transferred to a separatory funnel into which 100 mL of water had been put, and extracted three times with 100 mL of ethyl acetate. The organic layer was washed with water and dehydrated over anhydrous sodium sulfate. After filtering the drying agent off, the filtrate was concentrated and the residue was purified by silica gel column chromatography to obtain 9.74 g of a compound represented by Formula (11) as the intermediate compound 1-1.

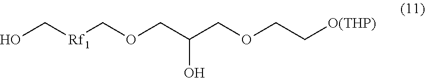

(in Formula (11), Rf₁ is the PFPE chain represented by Formula (4-1); in Rf₁, h indicating an average degree of polymerization represents 4.5 and i indicating an average degree of polymerization represents 4.5; and THP represents a tetrahydropyranyl group)

### (Second Reaction)

Subsequently, 9.61 g of the compound represented by Formula (11), which is the intermediate compound 1-1 obtained above, 0.75 g of the compound represented by Formula (7-1), and 20 mL of t-butanol were charged into a 100 mL eggplant flask in a nitrogen gas atmosphere, and stirred at room temperature until a homogenous mixture was obtained. 0.45 g of potassium tert-butoxide was added to the mixture and stirred at 70° C. for 16 hours to undergo a reaction. The compound represented by Formula (7-1) was synthesized by reacting one molecule of 1,3-propanediol with two molecules of epibromohydrin.

The reaction solution obtained after the reaction was cooled to room temperature, 50 g of a 10% hydrochloric acid/methanol solution (hydrochloric acid-methanol reagent (5% to 10%) manufactured by Tokyo Chemical Industry Co., Ltd.) was added thereto, and the mixture was stirred at room temperature for 4 hours. Thereafter, the reaction solution was gradually transferred to a separatory funnel into which 100 mL of a saturated aqueous sodium bicarbonate solution had been put, and then extracted twice with 200 mL of ethyl acetate. The organic layer was washed with 100 mL of saline, 100 mL of a saturated aqueous sodium bicarbonate solution, and 100 ml, of saline in this order, and then dehydrated over anhydrous sodium sulfate. After filtering the drying agent off, the filtrate was concentrated and the residue was purified by silica gel column chromatography to obtain 3.38 g of a compound (AA) (in Formula (AA), Rf₁ is the PFPE chain represented by Formula (4-1), and in the two Rf₁'s, h indicating an average degree of polymerization represents 4.5 and i indicating an average degree of polymerization represents 4.5).

The obtained compound (AA) was subjected to ¹H-NMR measurement and ¹⁹F-NMR measurement, and a structure thereof was identified based on the following results.

¹H-NMR (acetone-D₆): δ [ppm]=1.65 to 1.85 (2H), 3.40 to 3.85 (38H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 2

The compound represented by Formula (AB) was obtained by a method shown below.

The same operation as in Example 1, except that a compound represented by Formula (7-2) was used instead of the compound represented by Formula (7-1), was performed to obtain 3.43 g of the compound (AB) (in Formula (AB), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (7-2) was synthesized by reacting one molecule of ethylene glycol with two molecules of epibromohydrin.

The obtained compound (AB) was subjected to ¹H-NMR measurement and ¹⁹F-NMR measurement, and a structure thereof was identified based on the following results. ¹H-NMR (acetone-D₆): δ [ppm]=3.40 to 3.85 (38H), 3.85 to 4.10 (8H) ¹⁹F-NMR (acetone-D₆): δ [ppm]=−55.5 to −51.5 (18F), −78.5 (4F), −80.5 (4F), −91.0 to −88.5 (36F)

### Example 3

The compound represented by Formula (AC) was obtained by a method shown below.

The same operation as in Example 1, except that a compound represented by Formula (7-3) was used instead of the compound represented by Formula (7-1), was performed to obtain 3.43 g of the compound (AC) (in Formula (AC), Rf₁ is the PFPE chain represented by Formula (4-1), and in the two Rf₁'s, h indicating an average degree of polymerization represents 4.5 and i indicating an average degree of polymerization represents 4.5).

A commercially available product (manufactured by Tokyo Chemical Industry Co., Ltd.) was used as the compound represented by Formula (7-3).

The obtained compound (AC) was subjected to ¹H-NMR measurement and ¹⁹F-NMR measurement, and the structure was identified based on the following results.

¹H-NMR (acetone-D₆): δ [ppm]=1.65 to 1.85 (4H), 3.40 to 3.85 (38H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 4

The compound represented by Formula (AD) was obtained by a method shown below.

The same operation as in Example 1, except that a compound represented by Formula (7-4) was used instead of the compound represented by Formula (7-1), was performed to obtain 3.61 g of the compound (AD) (in Formula (AD), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (7-4) was synthesized by reacting one molecule of 1,6-hexanediol with two molecules of epibromohydrin.

The obtained compound (AD) was subjected to ¹H-NMR measurement and ¹⁹F-NMR measurement, and a structure thereof was identified based on the following results. ¹H-NMR (acetone-D₆): δ [ppm]=1.20 to 1.85 (8H), 3.40 to 3.85 (38H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 5

The compound represented by Formula (AE) was obtained by a method shown below.

The same operation as in Example 1, except that a compound represented by Formula (7-5) was used instead of the compound represented by Formula (7-1), was performed to obtain 3.68 g of the compound (AE) (in Formula (AE), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (7-5) was synthesized by reacting one molecule of 1,8-octanediol with two molecules of epibromohydrin.

The obtained compound (AE) was subjected to ¹H-NMR measurement and ¹⁹F-NMR measurement, and a structure thereof was identified based on the following results.

¹H-NMR (acetone-D₆): δ [ppm]=1.20 to 1.85 (12H), 3.40 to 3.85 (38H), 3.85 to 4.10 (8H)

¹⁹F-NMR (acetone-D₆): S [ppm]=−55.5 to −51.5 (18F), −78.5 (4F), −80.5 (4F), −91.0 to −88.5 (36F)

### Example 6

The compound represented by Formula (AF) was obtained by a method shown below.

The same operation as in Example 1, except that a compound represented by Formula (7-6) was used instead of the compound represented by Formula (7-1), was performed to obtain 3.52 g of the compound (AF) (in Formula (AF), Rf₁ is the PFPE chain represented by Formula (4-1), and in the two Rf₁'s, h indicating an average degree of polymerization represents 4.5 and i indicating an average degree of polymerization represents 4.5).

A commercially available product (manufactured by Tokyo Chemical Industry Co., Ltd.) was used as the compound represented by Formula (7-6).

The obtained compound (AF) was subjected to ¹H-NMR measurement and ¹⁹F-NMR measurement, and a structure thereof was identified based on the following results.

¹H-NMR (acetone-D₆): δ [ppm]=0.90 (6H), 3.40 to 3.85 (38H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 7

The compound represented by Formula (AG) was obtained by a method shown below.

The same operation as in Example 1, except that a compound represented by Formula (7-7) was used instead of the compound represented by Formula (7-1), was performed to obtain 3.21 g of the compound (AG) (in Formula (AG), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (7-7) was synthesized by reacting one molecule of 2,3-butanediol with two molecules of epibromohydrin.

The obtained compound (AG) was subjected to ¹H-NMR measurement and ¹⁹F-NMR measurement, and a structure thereof was identified based on the following results.

¹H-NMR (acetone-D₆): δ [ppm]=1.15 to 1.20 (6H), 3.40 to 3.85 (36H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 8

The compound represented by Formula (AH) was obtained by a method shown below.

The same operation as in Example 1, except that a compound represented by Formula (7-8) was used instead of the compound represented by Formula (7-1), was performed to obtain 3.41 g of the compound (AH) (in Formula (AH), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (7-8) was synthesized by reacting one molecule of dipropylene glycol with two molecules of epibromohydrin.

The obtained compound (AH) was subjected to ¹H-NMR measurement and ¹⁹F-NMR measurement, and a structure thereof was identified based on the following results.

¹H-NMR (acetone-D₆): δ [ppm]=1.65 to 1.85 (4H), 3.40 to 3.85 (42H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 9

The compound represented by Formula (AT) was obtained by a method shown below.

The same operation as in Example 1, except that a compound represented by Formula (7-9) was used instead of the compound represented by Formula (7-1), was performed to obtain 3.63 g of the compound (AT) (in Formula (AT), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (7-9) was synthesized by reacting one molecule of diethylene glycol with two molecules of epibromohydrin.

The obtained compound (Al) was subjected to ¹H-NMR measurement and ¹⁹F-NMR measurement, and a structure thereof was identified based on the following results.

¹H-NMR (acetone-D₆): δ [ppm]=3.40 to 3.85 (42H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 10

The compound represented by Formula (AJ) was obtained by a method shown below.

The same operation as in Example 1, except that a compound represented by Formula (7-10) was used instead of the compound represented by Formula (7-1) of Example 1, was performed to obtain 3.81 g of the compound (AJ) (in Formula (AJ), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (7-10) was synthesized by reacting one molecule of tetraethylene glycol and two molecules of epibromohydrin.

The obtained compound (AJ) was subjected to ¹H-NMR measurement and ¹⁹F-NMR measurement, and a structure thereof was identified based on the following results.

¹H-NMR (acetone-D₆): δ [ppm]=3.40 to 3.85 (50H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 11

The compound represented by Formula (BA) was obtained by a method shown below.

The same operation as in Example 1, except that a compound (number-average molecular weight: 1,000, molecular weight distribution: 1.1) represented by HOCH₂CF₂CF₂O(CF₂CF₂CF₂O)ⱼCF₂CF₂CH₂OH (in the formula, j indicating an average degree of polymerization is 4.5) was used instead of the compound represented by HOCH₂CF₂O(CF₂CF₂O)ₕ(CF₂O)ₕCF₂CH₂OH, was performed to obtain 3.49 g of the compound (BA) (in Formula (BA), Rf₂ is the PFPE chain represented by Formula (4-2), and in the two Rf₂'s, j indicating an average degree of polymerization represents 4.5).

The obtained compound (BA) was subjected to ¹H-NMR measurement and ¹⁹F-NMR measurement, and a structure thereof was identified based on the following results.

¹H-NMR (acetone-D₆): δ [ppm]=1.65 to 1.85 (2H), 3.40 to 3.85 (38H), 3.85 to 4.10 (8H)

¹⁹F-NMR (acetone-D₆): δ [ppm]=−84.0 to −83.0 (36F), −86.4 (8F), −124.3 (8F), −130.0 to −129.0 (18F)

### Example 12

The compound represented by Formula (BB) was obtained by a method shown below.

The same operation as in Example 1, except that a compound (number-average molecular weight: 1,000, molecular weight distribution: 1.1) represented by HOCH₂CF₂CF₂CF₂O(CF₂CF₂CF₂CF₂O)ₖCF₂CF₂CF₂CH₂OH (in the formula, k indicating an average degree of polymerization is 3.0) was used instead of the compound represented by HOCH₂CF₂O(CF₂CF₂O)ₕ(CF₂O)ₕCF₂CH₂OH, was performed to obtain 3.23 g of the compound (BB) (in Formula (BB), Rf₃ is the PFPE chain represented by Formula (4-3), and in the two Rf₃'s, k indicating an average degree of polymerization represents 3.0).

The obtained compound (BB) was subjected to ¹H-NMR measurement and ¹⁹F-NMR measurement, and a structure thereof was identified based on the following results.

¹H-NMR (acetone-D₆): δ [ppm]=1.65 to 1.85 (2H), 3.40 to 3.85 (38H), 3.85 to 4.10 (8H)

¹⁹F-NMR (acetone-D₆): δ [ppm]=−84.0 to −83.0 (32F), −122.5 (8F), −126.0 (24F), −129.0 to −128.0 (8F)

### Example 13

The compound represented by Formula (BC) was obtained by a method shown below.

The same operation as in Example 1, except that a compound represented by Formula (5-2) was used instead of the compound represented by Formula (5-1), was performed to obtain 3.45 g of the compound (BC) (in Formula (BC), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (5-2) was synthesized by protecting one of hydroxy groups of 1,3-propanediol using dihydropyran, and then reacting the product with epibromohydrin.

The obtained compound (BC) was subjected to ¹H-NMR measurement and ¹⁹F-NMR measurement, and a structure thereof was identified based on the following results.

¹H-NMR (acetone-D₆): δ [ppm]=1.65 to 1.85 (6H), 3.40 to 3.85 (38H), 3.85 to 4.10 (8H)

¹⁹F-NMR (acetone-D₆): S [ppm]=−55.5 to −51.5 (18F), −78.5 (4F), −80.5 (4F), −91.0 to −88.5 (36F)

### Example 14

The compound represented by Formula (BD) was obtained by a method shown below.

The same operation as in Example 1, except that a compound represented by Formula (5-3) was used instead of the compound represented by Formula (5-1), was performed to obtain 3.12 g of the compound (BD) (in Formula (BD), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (5-3) was synthesized by protecting 3-buten-1-ol with dihydropyran, and then oxidizing the product with m-chloroperbenzoic acid.

The obtained compound (BD) was subjected to ¹H-NMR measurement and ¹⁹F-NMR measurement, and a structure thereof was identified based on the following results.

¹H-NMR (acetone-D₆): δ [ppm]=1.65 to 1.85 (6H), 3.40 to 3.85 (30H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 15

The compound represented by Formula (BE) was obtained by a method shown below.

The same operation as in Example 1, except that a compound represented by Formula (5-4) was used instead of the compound represented by Formula (5-1) of Example 1, was performed to obtain 3.34 g of the compound (BE) (in Formula (BE), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (5-4) was synthesized by reacting 3-buten-1-ol with 2-(2-bromoethoxy)tetrahydro-2H-pyran, and then oxidizing the product with m-chloroperbenzoic acid.

The obtained compound (BE) was subjected to ¹H-NMR measurement and ¹⁹F-NMR measurement, and a structure thereof was identified based on the following results.

¹H-NMR (acetone-D₆): δ [ppm]=1.65 to 1.85 (6H), 3.40 to 3.85 (38H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 16

The compound represented by Formula (BF) was obtained by a method shown below.

The same operation as in Example 1, except that a compound represented by Formula (5-5) was used instead of the compound represented by Formula (5-1), was performed to obtain 3.71 g of the compound (BF) (in Formula (BF), Rf₁ is the PFPE chain represented by Formula (4-1), and in the two Rf₁'s, h indicating an average degree of polymerization represents 4.5 and i indicating an average degree of polymerization represents 4.5).

Furthermore, the compound represented by Formula (5-5) was synthesized by subjecting the compound represented by Formula (5-1) and an allyl alcohol to an addition reaction, then protecting the product with dihydropyran, and further oxidizing the product with m-chloroperbenzoic acid.

The obtained compound (BF) was subjected to ¹H-NMR measurement and ¹⁹F-NMR measurement, and a structure thereof was identified based on the following results.

¹H-NMR (acetone-D₆): δ [ppm]=1.65 to 1.85 (2H), 3.40 to 3.85 (50H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 17

The compound represented by Formula (BG) was obtained by a method shown below.

The same operation as in Example 1, except that a compound represented by Formula (5-6) was used instead of the compound represented by Formula (5-1), was performed to obtain 3.39 g of the compound (BG) (in Formula (BG), Rf₁ is the PFPE chain represented by Formula (4-1), and in the two Rf₁'s, h indicating an average degree of polymerization represents 4.5 and i indicating an average degree of polymerization represents 4.5).

Furthermore, the compound represented by Formula (5-6) was synthesized by subjecting the compound represented by Formula (5-3) and an allyl alcohol to an addition reaction, then protecting the product with dihydropyran, and further oxidizing the product with m-chloroperbenzoic acid.

The obtained compound (BG) was subjected to ¹H-NMR measurement and ¹⁹F-NMR measurement, and a structure thereof was identified based on the following results.

¹H-NMR (acetone-D₆): δ [ppm]=1.65 to 1.85 (6H), 3.40 to 3.85 (42H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 18

The compound represented by Formula (BH) was obtained by a method shown below.

The same operation as in Example 1, except that a compound represented by Formula (5-7) was used instead of the compound represented by Formula (5-1), was performed to obtain 3.52 g of the compound (BH) (in Formula (BH), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (5-7) was synthesized by subjecting the compound represented by Formula (5-1) and 3-buten-1-ol to an addition reaction, protecting the product with dihydropyran, and further oxidizing the product with m-chloroperbenzoic acid.

The obtained compound (BH) was subjected to ¹H-NMR measurement and ¹⁹F-NMR measurement, and a structure thereof was identified based on the following results.

¹H-NMR (acetone-D₆): δ [ppm]=1.65 to 1.85 (6H), 3.40 to 3.85 (50H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 19

The compound represented by Formula (BI) was obtained by a method shown below.

### (First Reaction)

The compound represented by Formula (11) was obtained as the intermediate compound 1a in the same manner as in the first reaction of Example 1.

### (Second Reaction)

The same operation as the first reaction of Example 1, except that a compound represented by Formula (5-5) was used instead of the compound represented by Formula (5-1), was performed to obtain a compound represented by Formula (12) as the intermediate compound 1b.

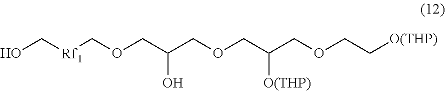

(in Formula (12), Rf₁ is the PFPE chain represented by Formula (4-1); in Rf₁, h indicating an average degree of polymerization represents 4.5, i indicating an average degree of polymerization represents 4.5; and THP represents a tetrahydropyranyl group)

(Third reaction)

Subsequently, 9.66 g of the compound represented by Formula (11), which is the intermediate compound 1a obtained above, 0.69 g of the compound represented by Formula (9-1), and 20 mL of t-butanol were charged into a 100 mL eggplant flask in a nitrogen gas atmosphere, and stirred at room temperature until a homogenous mixture was obtained. 0.45 g of potassium tert-butoxide was added to the mixture and stirred at 70° C. for 16 hours to undergo a reaction. The compound represented by Formula (9-1) was synthesized by reacting allyl bromide and epibromohydrin in this order with 1,3-propanediol.

The reaction solution obtained above was cooled to 25° C., transferred to a separatory funnel into which 100 mL of water had been put, and extracted three times with 100 mL of ethyl acetate. The organic layer was washed with water and dehydrated over anhydrous sodium sulfate. After filtering the drying agent off, the filtrate was concentrated and the residue was oxidized with m-chloroperbenzoic acid and then purified by silica gel column chromatography to obtain 7.58 g of a compound represented by Formula (13) as the intermediate compound 1-2.

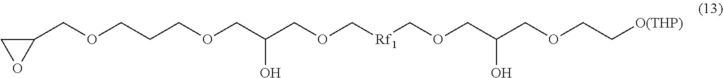

(in Formula (13), Rf₁ is the PFPE chain represented by Formula (4-1); in Rf₁, h indicating an average degree of polymerization represents 4.5 and i indicating an average degree of polymerization represents 4.5; and THP represents a tetrahydropyranyl group)

(Fourth reaction)

Subsequently, 7.35 g of the compound represented by Formula (12), which is the intermediate compound 1b obtained above, 7.58 g of the compound represented by Formula (13), which is the intermediate compound 1-2 obtained above, and 20 mL of t-butanol were charged into a 100 mL eggplant flask in a nitrogen gas atmosphere, and stirred at room temperature until a homogenous mixture was obtained. 0.45 g of potassium tert-butoxide was added to the mixture and stirred at 70° C. for 16 hours to undergo a reaction.

The reaction solution obtained after the reaction was cooled to room temperature, 50 g of a 10% hydrochloric acid/methanol solution (hydrochloric acid-methanol reagent (5% to 10%) manufactured by Tokyo Chemical Industry Co., Ltd.) was added thereto, and the mixture was stirred at room temperature for 4 hours. Thereafter, the reaction solution was gradually transferred to a separatory funnel into which 100 mL of a saturated aqueous sodium bicarbonate solution had been put, and then extracted twice with 200 mL of ethyl acetate. The organic layer was washed with 100 mL of saline, 100 mL of a saturated aqueous sodium bicarbonate solution, and 100 mL of saline in this order, and then dehydrated over anhydrous sodium sulfate. After filtering the drying agent off, the filtrate was concentrated and the residue was purified by silica gel column chromatography to obtain 4.54 g of a compound (BI) (in Formula (BI), Rf₁ is the PFPE chain represented by Formula (4-1), and in the two Rf₁'s, h indicating an average degree of polymerization represents 4.5 and i indicating an average degree of polymerization represents 4.5).

The obtained compound (BI) was subjected to ¹H-NMR measurement and ¹⁹F-NMR measurement, and a structure thereof was identified based on the following results.

¹H-NMR (acetone-D₆): δ [ppm]=1.65 to 1.85 (2H), 3.40 to 3.85 (44H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 20

The compound represented by Formula (BJ) was obtained by a method shown below.

The same operation as in Example 1, except that a compound represented by Formula (5-8) was used instead of the compound represented by Formula (5-1), was performed to obtain 3.67 g of the compound (BJ) (in Formula (BJ), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (5-8) was synthesized by protecting one of hydroxy groups of 1,6-hexanediol using dihydropyran, and then reacting the product with epibromohydrin.

The obtained compound (BJ) was subjected to ¹H-NMR measurement and ¹⁹F-NMR measurement, and a structure thereof was identified based on the following results.

¹H-NMR (acetone-D₆): δ [ppm]=1.20 to 1.85 (18H), 3.40 to 3.85 (38H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 21

The compound represented by Formula (BK) was obtained by a method shown below.

The same operation as in Example 1, except that a compound represented by Formula (5-9) was used instead of the compound represented by Formula (5-1), was performed to obtain 3.30 g of the compound (BK) (in Formula (BK), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (5-9) was synthesized by protecting 7-octen-1-ol with dihydropyran, and then oxidizing the product with m-chloroperbenzoic acid.

The obtained compound (BK) was subjected to ¹H-NMR measurement and ¹⁹F-NMR measurement, and a structure thereof was identified based on the following results.

¹H-NMR (acetone-D₆): δ [ppm]=1.20 to 1.85 (18H), 3.40 to 3.85 (30H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 22

The compound represented by Formula (BL) was obtained by a method shown below.

The same operation as in Example 1, except that a compound represented by Formula (5-10) was used instead of the compound represented by Formula (5-1), was performed to obtain 2.95 g of the compound (BL) (in Formula (BL), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (5-10) was synthesized by protecting 2-bromoethanol with dihydropyran, further reacting 5-hexen-1-ol therewith, and then oxidizing the product with m-chloroperbenzoic acid.

The obtained compound (BL) was subjected to ¹H-NMR measurement and ¹⁹F-NMR measurement, and a structure thereof was identified based on the following results.

¹H-NMR (acetone-D₆): δ [ppm]=1.20 to 1.85 (14H), 3.40 to 3.85 (38H), 3.85 to 4.10 (8H)

¹⁹F-NMR (acetone-D₆): S [ppm]=−55.5 to −51.5 (18F), −78.5 (4F), −80.5 (4F), −91.0 to −88.5 (36F)

### Example 23

The compound represented by Formula (BM) was obtained by a method shown below.

The same operation as in Example 1, except that a compound represented by Formula (5-11) was used instead of the compound represented by Formula (5-1), was performed to obtain 3.01 g of the compound (BM) (in Formula (BM), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (5-11) was synthesized by protecting allyl alcohol with dihydropyran, and then oxidizing the product with m-chloroperbenzoic acid.

The obtained compound (BM) was subjected to ¹H-NMR measurement and ¹⁹F-NMR measurement, and a structure thereof was identified based on the following results.

¹H-NMR (acetone-D₆): δ [ppm]=1.65 to 1.85 (2H), 3.40 to 3.85 (30H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 24

The compound represented by Formula (BN) was obtained by a method shown below.

The same operation as in Example 1, except that a compound represented by Formula (5-12) was used instead of the compound represented by Formula (5-1), was performed to obtain 3.08 g of the compound (BN) (in Formula (BN), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (5-12) was synthesized by protecting two hydroxy groups of 1-allyloxy-2,3-propanediol with dihydropyran, and then oxidizing the product with m-chloroperbenzoic acid.

The obtained compound (BN) was subjected to ¹H-NMR measurement and ¹⁹F-NMR measurement, and a structure thereof was identified based on the following results.

¹H-NMR (acetone-D₆): δ [ppm]=1.65 to 1.85 (2H), 3.40 to 3.85 (42H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 25

The compound represented by Formula (BO) was obtained by a method shown below.

The same operation as in Example 1, except that a compound represented by Formula (5-13) was used instead of the compound represented by Formula (5-1), was performed to obtain 3.31 g of the compound (BO) (in Formula (BO), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (5-13) was synthesized by adding allyl alcohol to allyl glycidyl ether, protecting the generated hydroxy group using dihydropyran, and then oxidizing one of allyl groups with m-chloroperbenzoic acid.

The obtained compound (BO) was subjected to ¹H-NMR measurement and ¹⁹F-NMR measurement, and a structure thereof was identified based on the following results.

¹H-NMR (acetone-D₆): δ [ppm]=1.65 to 1.85 (2H), 3.40 to 3.85 (44H), 3.85 to 4.10 (8H), 5.40 to 6.10 (6H)

¹⁹F-NMR (acetone-D₆): δ [ppm]=−55.5 to −51.5 (18F), −78.5 (4F), −80.5 (4F), −91.0 to −88.5 (36F)

### Example 26

The compound represented by Formula (BP) was obtained by a method shown below.

The same operation as in Example 1, except that a compound represented by Formula (5-14) was used instead of the compound represented by Formula (5-1), was performed to obtain 3.28 g of the compound (BP) (in Formula (BP), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (5-14) was synthesized by reacting p-methoxyphenol with epibromohydrin.

The obtained compound (BP) was subjected to ¹H-NMR measurement and ¹⁹F-NMR measurement, and a structure thereof was identified based on the following results.

¹H-NMR (acetone-D₆): δ [ppm]=1.65 to 1.85 (2H), 3.40 to 3.85 (34H), 3.85 to 4.10 (8H), 6.30 to 7.10 (8H)

¹⁹F-NMR (acetone-D₆): δ [ppm]=−55.5 to −51.5 (18F), −78.5 (4F), −80.5 (4F), −91.0 to −88.5 (36F)

### Example 27

The compound represented by Formula (BQ) was obtained by a method shown below.

The same operation as in Example 1, except that a compound represented by Formula (5-15) was used instead of the compound represented by Formula (5-1) of Example 1, was performed to obtain 3.36 g of the compound (BQ) (in Formula (BQ), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (5-15) was synthesized by reacting N-(2-hydroxyethyl)acetamide with epibromohydrin.

The obtained compound (BQ) was subjected to ¹H-NMR measurement and ¹⁹F-NMR measurement, and a structure thereof was identified based on the following results.

¹H-NMR (acetone-D₆): δ [ppm]=1.65 to 1.85 (2H), 1.90 (6H), 3.40 to 3.85 (36H), 3.85 to 4.10 (8H), 6.30 to 6.50 (2H)

¹⁹F-NMR (acetone-D₆): δ [ppm]=−55.5 to −51.5 (18F), −78.5 (4F), −80.5 (4F), −91.0 to −88.5 (36F)

### Example 28

The compound represented by Formula (BR) was obtained by a method shown below.

The same operation as in Example 1, except that a compound represented by Formula (5-16) was used instead of the compound represented by Formula (5-1) of Example 1, was performed to obtain 3.57 g of the compound (BR) (in Formula (BR), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (5-16) was synthesized by reacting 2-cyanoethanol with epibromohydrin.

The obtained compound (BR) was subjected to ¹H-NMR measurement and ¹⁹F-NMR measurement, and a structure thereof was identified based on the following results.

¹H-NMR (acetone-D₆): δ [ppm]=1.65 to 1.85 (2H), 2.00 to 2.10 (4H), 3.40 to 3.85 (32H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 29

The compound represented by Formula (CA) was obtained by a method shown below.

The same operation as in Example 1, except that a compound represented by Formula (14) was used instead of the compound represented by Formula (7-1), was performed to obtain 3.81 g of the compound (CA) (in Formula (CA), Rf₁ is the PFPE chain represented by Formula (4-1), and in the three Rf₁'s, h indicating an average degree of polymerization represents 4.5 and i indicating an average degree of polymerization represents 4.5).

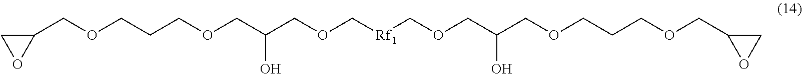

(in Formula (14), Rf₁ is the PFPE chain represented by Formula (4-1); and in Rf₁, h indicating an average degree of polymerization represents 4.5 and i indicating an average degree of polymerization represents 4.5)

The compound represented by Formula (14) was synthesized by reacting a compound (number-average molecular weight: 1,000, molecular weight distribution: 1.1) represented by HOCH₂CF₂O(CF₂CF₂O)ᵢ, (CF₂O);CF₂CH₂OH (in the formula, h indicating an average degree of polymerization is 4.5 and i indicating an average degree of polymerization is 4.5) with the compound represented by Formula (9-1), and then oxidizing the product with m-chloroperbenzoic acid.

The obtained compound (CA) was subjected to ¹H-NMR measurement and ¹⁹F-NMR measurement, and a structure thereof was identified based on the following results.

¹H-NMR (acetone-D₆): δ [ppm]=1.65 to 1.85 (4H), 3.40 to 3.85 (54H), 3.85 to 4.10 (12H)

¹⁹F-NMR (acetone-D₆): δ [ppm]=−55.5 to −51.5 (27F), −78.5 (6F), −80.5 (6F), −91.0 to −88.5 (54F)

### Example 30

The compound represented by Formula (CB) was obtained by a method shown below.

The same operation as in Example 29, except that a compound represented by Formula (9-2) was used instead of the compound represented by Formula (9-1), was performed to obtain 4.11 g of the compound (CB) (in Formula (CB), Rf₁ is the PFPE chain represented by Formula (4-1), and in the three Rf₁'s, h indicating an average degree of polymerization represents 4.5 and i indicating an average degree of polymerization represents 4.5).

The compound represented by Formula (9-2) was synthesized by reacting allyl bromide and epibromohydrin in this order with ethylene glycol.

The obtained compound (CB) was subjected to ¹H-NMR measurement and ¹⁹F-NMR measurement, and a structure thereof was identified based on the following results.

¹H-NMR (acetone-D₆): δ [ppm]=3.40 to 3.85 (54H), 3.85 to 4.10 (12H)

¹⁹F-NMR (acetone-D₆): δ [ppm]=−55.5 to −51.5 (27F), −78.5 (6F), −80.5 (6F), −91.0 to −88.5 (54F)

### Example 31

The compound represented by Formula (CC) was obtained by a method shown below.

The same operation as in Example 29, except that a compound represented by Formula (9-5) was used instead of the compound represented by Formula (9-1), was performed to obtain 4.32 g of the compound (CC) (in Formula (CC), Rf₁ is the PFPE chain represented by Formula (4-1), and in the three Rf₁'s, h indicating an average degree of polymerization represents 4.5 and i indicating an average degree of polymerization represents 4.5).

The compound represented by Formula (9-5) was synthesized by reacting allyl bromide and epibromohydrin with 1,8-octanediol in this order.

The obtained compound (CC) was subjected to ¹H-NMR measurement and ¹⁹F-NMR measurement, and a structure thereof was identified based on the following results.

¹H-NMR (acetone-D₆): δ [ppm]=1.20 to 1.85 (24H), 3.40 to 3.85 (54H), 3.85 to 4.10 (12H)

¹⁹F-NMR (acetone-D₆): δ [ppm]=−55.5 to −51.5 (27F), −78.5 (6F), −80.5 (6F), −91.0 to −88.5 (54F)

### Example 32

The compound represented by Formula (CD) was obtained by a method shown below.

The same operation as in Example 29, except that a compound represented by Formula (9-6) was used instead of the compound represented by Formula (9-1), was performed to obtain 4.21 g of a compound (CD) (in Formula (CD), Rf₁ is the PFPE chain represented by Formula (4-1), and in the three Rf₁'s, h indicating an average degree of polymerization represents 4.5 and i indicating an average degree of polymerization represents 4.5).

The compound represented by Formula (9-6) was synthesized by reacting 2,2-dimethyl-1,3-propanediol with allyl bromide and epibromohydrin in this order.

The obtained compound (CD) was subjected to ¹H-NMR measurement and ¹⁹F-NMR measurement, and a structure thereof was identified based on the following results.

¹H-NMR (acetone-D₆): δ [ppm]=0.90 (12H), 3.40 to 3.85 (54H), 3.85 to 4.10 (12H)

¹⁹F-NMR (acetone-D₆): δ [ppm]=−55.5 to −51.5 (27F), −78.5 (6F), −80.5 (6F), −91.0 to −88.5 (54F)

### Example 33

The compound represented by Formula (CE) was obtained by a method shown below.

The same operation as in Example 29, except that a compound represented by Formula (9-7) was used instead of the compound represented by Formula (9-1), was performed to obtain 3.96 g of the compound (CE) (in Formula (CE), Rf₁ is the PFPE chain represented by Formula (4-1), and in the three Rf₁'s, h indicating an average degree of polymerization represents 4.5 and i indicating an average degree of polymerization represents 4.5).

The compound represented by Formula (9-7) was synthesized by reacting allyl bromide and epibromohydrin in this order with 2,3-butanediol.

The obtained compound (CE) was subjected to ¹H-NMR measurement and ¹⁹F-NMR measurement, and a structure thereof was identified based on the following results.

¹H-NMR (acetone-D₆): δ [ppm]=1.15 to 1.20 (12H), 3.40 to 3.85 (50H), 3.85 to 4.10 (12H)

¹⁹F-NMR (acetone-D₆): δ [ppm]=−55.5 to −51.5 (27F), −78.5 (6F), −80.5 (6F), −91.0 to −88.5 (54F)

### Example 34

The compound represented by Formula (CF) was obtained by a method shown below.

The same operation as in Example 29, except that a compound represented by Formula (9-9) was used instead of the compound represented by Formula (9-1), was performed to obtain 4.35 g of a compound (CF) (in Formula (CF), Rf₁ is the PFPE chain represented by Formula (4-1), and in the three Rf₁'s, h indicating an average degree of polymerization represents 4.5 and i indicating an average degree of polymerization represents 4.5).

The compound represented by Formula (9-9) was synthesized by reacting allyl bromide and epibromohydrin in this order with diethylene glycol.

The obtained compound (CF) was subjected to ¹H-NMR measurement and ¹⁹F-NMR measurement, and a structure thereof was identified based on the following results.

¹H-NMR (acetone-D₆): δ [ppm]=3.40 to 3.85 (62H), 3.85 to 4.10 (12H)

¹⁹F-NMR (acetone-D₆): δ [ppm]=−55.5 to −51.5 (27F), −78.5 (6F), −80.5 (6F), −91.0 to −88.5 (54F)

### Example 35

The compound represented by Formula (DA) was obtained by a method shown below.

The same operation as in Example 29, except that in the synthesis of the compound represented by Formula (11) and the compound represented by Formula (14), a compound (number-average molecular weight: 1,000, molecular weight distribution: 1.1) represented by HOCH₂CF₂CF₂O(CF₂CF₂CF₂O)ⱼCF₂CF₂CH₂OH (in the formula, j indicating an average degree of polymerization is 4.5) was used instead of the compound represented by HOCH₂CF₂O(CF₂CF₂O)ₕ(CF₂O)ₕCF₂CH₂OH, was performed to obtain 4.25 g of the compound (DA) (in Formula (DA), Rf₂ is the PFPE chain represented by Formula (4-2), and in the three Rf₂'s, j indicating an average degree of polymerization represents 4.5).

The obtained compound (DA) was subjected to ¹H-NMR measurement and ¹⁹F-NMR measurement, and a structure thereof was identified based on the following results.

¹H-NMR (acetone-D₆): δ [ppm]=1.65 to 1.85 (4H), 3.40 to 3.85 (54H), 3.85 to 4.10 (12H)

¹⁹F-NMR (acetone-D₆): δ [ppm]=−84.0 to −83.0 (54F), −86.4 (12F), −124.3 (12F), −130.0 to −129.0 (27F)

### Example 36

The compound represented by Formula (DB) was obtained by a method shown below.

The same operation as in Example 29, except that a compound represented by Formula (5-5) was used instead of the compound represented by Formula (5-1), was performed to obtain 4.41 g of a compound (DB) (in Formula (DB), Rf₁ is the PFPE chain represented by Formula (4-1), and in the three Rf₁'s, h indicating an average degree of polymerization represents 4.5 and i indicating an average degree of polymerization represents 4.5).

The obtained compound (DB) was subjected to ¹H-NMR measurement and ¹⁹F-NMR measurement, and a structure thereof was identified based on the following results.

¹H-NMR (acetone-D₆): δ [ppm]=1.65 to 1.85 (4H), 3.40 to 3.85 (66H), 3.85 to 4.10(12H)

¹⁹F-NMR (acetone-D₆): δ [ppm]=−55.5 to −51.5 (27F), −78.5 (6F), −80.5 (6F), −91.0 to −88.5 (54F)

### Example 37

The compound represented by Formula (DC) was obtained by a method shown below.

The same operation as in Example 29, except that a compound represented by Formula (5-3) was used instead of the compound represented by Formula (5-1), was performed to obtain 3.81 g of a compound (DC) (in Formula (DC), Rf₁ is the PFPE chain represented by Formula (4-1), and in the three Rf₁'s, h indicating an average degree of polymerization represents 4.5 and i indicating an average degree of polymerization represents 4.5).

The obtained compound (DC) was subjected to ¹H-NMR measurement and ¹⁹F-NMR measurement, and a structure thereof was identified based on the following results.

¹H-NMR (acetone-D₆): δ [ppm]=1.65 to 1.85 (8H), 3.40 to 3.85 (46H), 3.85 to 4.10 (12H)

¹⁹F-NMR (acetone-D₆): S [ppm]=−55.5 to −51.5 (27F), −78.5 (6F), −80.5 (6F), −91.0 to −88.5 (54F)

### Example 38

The compound represented by Formula (DD) was obtained by a method shown below.

The same operation as in Example 29, except that a compound represented by Formula (5-4) was used instead of the compound represented by Formula (5-1), was performed to obtain 4.07 g of the compound (DD) (in Formula (DD), Rf₁ is the PFPE chain represented by Formula (4-1), and in the three Rf₁'s, h indicating an average degree of polymerization represents 4.5 and i indicating an average degree of polymerization represents 4.5).

The obtained compound (DD) was subjected to ¹H-NMR measurement and ¹⁹F-NMR measurement, and a structure thereof was identified based on the following results.

¹H-NMR (acetone-D₆): δ [ppm]=1.65 to 1.85 (8H), 3.40 to 3.85 (54H), 3.85 to 4.10 (12H)

¹⁹F-NMR (acetone-D₆): δ [ppm]=−55.5 to −51.5 (27F), −78.5 (6F), −80.5 (6F), −91.0 to −88.5 (54F)

### Example 39

The compound represented by Formula (DE) was obtained by a method shown below.

The same operation as in Example 29, except that a compound represented by Formula (5-15) was used instead of the compound represented by Formula (5-1), was performed to obtain 4.18 g of the compound (DE) (in Formula (DE), Rf₁ is the PFPE chain represented by Formula (4-1), and in the three Rf₁'s, h indicating an average degree of polymerization represents 4.5 and i indicating an average degree of polymerization represents 4.5).

The obtained compound (DE) was subjected to ¹H-NMR measurement and ¹⁹F-NMR measurement, and a structure thereof was identified based on the following results.

¹H-NMR (acetone-D₆): δ [ppm]=1.65 to 1.85 (4H), 1.90 (6H), 3.40 to 3.85 (52H), 3.85 to 4.10 (12H), 6.30 to 6.50 (2H)

¹⁹F-NMR (acetone-D₆): δ [ppm]=−55.5 to −51.5 (27F), −78.5 (6F), −80.5 (6F), −91.0 to −88.5 (54F)

### Example 40

The compound represented by Formula (DF) was obtained by a method shown below.

The same operation as in Example 29, except that a compound represented by Formula (5-16) was used instead of the compound represented by Formula (5-1), was performed to obtain 4.32 g of the compound (DF) (in Formula (DF), Rf₁ is the PFPE chain represented by Formula (4-1), and in the three Rf₁'s, h indicating an average degree of polymerization represents 4.5 and i indicating an average degree of polymerization represents 4.5).

The obtained compound (DF) was subjected to ¹H-NMR measurement and ¹⁹F-NMR measurement, and a structure thereof was identified based on the following results.

¹H-NMR (acetone-D₆): δ [ppm]=1.65 to 1.85 (4H), 2.00 to 2.10 (4H), 3.40 to 3.85 (48H), 3.85 to 4.10 (12H)

¹⁹F-NMR (acetone-D₆): δ [ppm]=−55.5 to −51.5 (27F), −78.5 (6F), −80.5 (6F), −91.0 to −88.5 (54F)

### Example 41

The compound represented by Formula (DG) was obtained by a method shown below.

The same operation as in Example 29, except that a compound represented by Formula (5-11) was used instead of the compound represented by Formula (5-1), was performed to obtain 3.86 g of the compound (DG) (in Formula (DG), Rf₁ is the PFPE chain represented by Formula (4-1), and in the three Rf₁'s, h indicating an average degree of polymerization represents 4.5 and i indicating an average degree of polymerization represents 4.5).

The obtained compound (DG) was subjected to ¹H-NMR measurement and ¹⁹F-NMR measurement, and a structure thereof was identified based on the following results.

¹H-NMR (acetone-D₆): δ [ppm]=1.65 to 1.85 (4H), 3.40 to 3.85 (46H), 3.85 to 4.10 (12H)

¹⁹F-NMR (acetone-D₆): δ [ppm]=−55.5 to −51.5 (27F), −78.5 (6F), −80.5 (6F), −91.0 to −88.5 (54F)

The values of x and the structures of R¹, R², R³, and R⁴ in a case where the compounds (AA) to (AJ), (BA) to (BR), (CA) to (CF), and (DA) to (DG) of Examples 1 to 41 obtained as described above were each applied to Formula (1) are shown in Table 1. Furthermore, the structures of R¹ and R⁴ are the same in all compounds other than the compound (BJ).

TABLE 1

| Compound | x | R¹ and R⁴ | | R² | R³ | |
|---|---|---|---|---|---|---|
| (AA) | 1 | (3-1) | p = 0/q = 0/r = 1/B = hydroxy group | (4-1) | (2-1) | a = 3 |
| (AB) | 1 | (3-1) | p = 0/q = 0/r = 1/B = hydroxy group | (4-1) | (2-1) | a = 2 |
| (AC) | 1 | (3-1) | p = 0/q = 0/r = 1/B = hydroxy group | (4-1) | (2-1) | a = 4 |
| (AD) | 1 | (3-1) | p = 0/q = 0/r = 1/B = hydroxy group | (4-1) | (2-1) | a = 6 |
| (AE) | 1 | (3-1) | p = 0/q = 0/r = 1/B = hydroxy group | (4-1) | (2-1) | a = 8 |
| (AF) | 1 | (3-1) | p = 0/q = 0/r = 1/B = hydroxy group | (4-1) | (2-1) | a = 3 |
| (AG) | 1 | (3-1) | p = 0/q = 0/r = 1/B = hydroxy group | (4-1) | (2-1) | a = 2 |
| (AH) | 1 | (3-1) | p = 0/q = 0/r = 1/B = hydroxy group | (4-1) | (2-2) | b = 2 |
| (AI) | 1 | (3-1) | p = 0/q = 0/r = 1/B = hydroxy group | (4-1) | (2-2) | b = 2 |
| (AJ) | 1 | (3-1) | p = 0/q = 0/r = 1/B = hydroxy group | (4-1) | (2-2) | b = 4 |
| (BA) | 1 | (3-1) | p = 0/q = 0/r = 1/B = hydroxy group | (4-2) | (2-1) | a = 3 |
| (BB) | 1 | (3-1) | p = 0/q = 0/r = 1/B = hydroxy group | (4-3) | (2-1) | a = 3 |
| (BC) | 1 | (3-1) | p = 0/q = 0/r = 2/B = hydroxy group | (4-1) | (2-1) | a = 3 |
| (BD) | 1 | (3-2) | s = 0/t = 1 | (4-1) | (2-1) | a = 3 |
| (BE) | 1 | (3-1) | p = 1/q = 0/r = 1/B = hydroxy group | (4-1) | (2-1) | a = 3 |
| (BF) | 1 | (3-1) | p = 0/q = 1/r = 1/B = hydroxy group | (4-1) | (2-1) | a = 3 |
| (BG) | 1 | (3-2) | s = 1/t = 1 | (4-1) | (2-1) | a = 3 |
| (BH) | 1 | (3-1) | p = 1/q = 1/r = 1/B = hydroxy group | (4-1) | (2-1) | a = 3 |
| (BI) | 1 | (3-1) | R¹: p = 0/q = 0/r = 1/B = hydroxy group | (4-1) | (2-1) | a = 3 |
| | | | R⁴: p = 0/q = 1/r = 1/B = hydroxy group | | | |
| (BJ) | 1 | (3-1) | p = 0/q = 0/r = 5/B = hydroxy group | (4-1) | (2-1) | a = 3 |
| (BK) | 1 | (3-2) | s = 0/t = 5 | (4-1) | (2-1) | a = 3 |
| (BL) | 1 | (3-1) | p = 3/q = 0/r = 1/B = hydroxy group | (4-1) | (2-1) | a = 3 |
| (BM) | 1 | (3) | l = 1/m = 1/n = 1/A = hydrogen atom | (4-1) | (2-1) | a = 3 |
| (BN) | 1 | (3) | l = 2/m = 1/n = 1/A = hydrogen atom | (4-1) | (2-1) | a = 3 |
| (BO) | 1 | (3) | l = 2/m = 1/n = 1/A = allyl group | (4-1) | (2-1) | a = 3 |
| (BP) | 1 | (3) | l = 1/m = 1/n = 1/A = p-methoxyphenyl group | (4-1) | (2-1) | a = 3 |
| (BQ) | 1 | (3-1) | p = 0/q = 0/r = 1/B = acetamide group | (4-1) | (2-1) | a = 3 |
| (BR) | 1 | (3-1) | p = 0/q = 0/r = 1/B = cyano group | (4-1) | (2-1) | a = 3 |

TABLE 2

| Compound | x | R¹ and R⁴ | | R² | R³ | |
|---|---|---|---|---|---|---|
| (CA) | 2 | (3-1) | p = 0/q = 0/r = 1/B = hydroxy group | (4-1) | (2-1) | a = 3 |
| (CB) | 2 | (3-1) | p = 0/q = 0/r = 1/B = hydroxy group | (4-1) | (2-1) | a = 2 |
| (CC) | 2 | (3-1) | p = 0/q = 0/r = 1/B = hydroxy group | (4-1) | (2-1) | a = 8 |
| (CD) | 2 | (3-1) | p = 0/q = 0/r = 1/B = hydroxy group | (4-1) | (2-1) | a = 3 |
| (CE) | 2 | (3-1) | p = 0/q = 0/r = 1/B = hydroxy group | (4-1) | (2-1) | a = 2 |
| (CF) | 2 | (3-1) | p = 0/q = 0/r = 1/B = hydroxy group | (4-1) | (2-2) | b = 2 |
| (DA) | 2 | (3-1) | p = 0/q = 0/r = 1/B = hydroxy group | (4-2) | (2-1) | a = 3 |
| (DB) | 2 | (3-1) | p = 0/q = 1/r = 1/B = hydroxy group | (4-1) | (2-1) | a = 3 |
| (DC) | 2 | (3-2) | s = 0/t = 1 | (4-1) | (2-1) | a = 3 |
| (DD) | 2 | (3-1) | p = 1/q = 0/r = 1/B = hydroxy group | (4-1) | (2-1) | a = 3 |
| (DE) | 2 | (3-1) | p = 0/q = 0/r = 1/B = acetamide group | (4-1) | (2-1) | a = 3 |
| (DF) | 2 | (3-1) | p = 0/q = 0/r = 1/B = cyano group | (4-1) | (2-1) | a = 3 |
| (DG) | 2 | (3) | l = 1/m = 1/n = 1/A = hydrogen atom | (4-1) | (2-1) | a = 3 |

### Comparative Example 1

A compound represented by Formula (ZA) was synthesized by the method described in Patent Document 1.

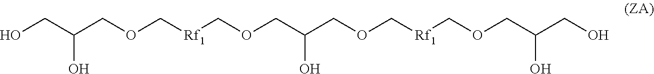

(in Formula (ZA), Rfₜ is the PFPE chain represented by Formula (4-1); and in the two Rf₁'s, h indicating an average degree of polymerization represents 7.0 and i indicating an average degree of polymerization represents 0)

### Comparative Example 2

A compound represented by Formula (ZB) was synthesized by the method described in Patent Document 2.

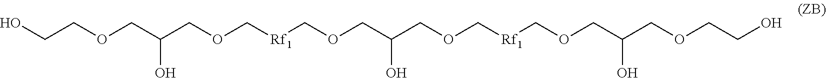

(in Formula (ZB), Rf₁ is the PFPE chain represented by Formula (4-1); and in the two Rf₁'s, h indicating an average degree of polymerization represents 4.5 and i indicating an average degree of polymerization represents 4.5)

### Comparative Example 3

A compound represented by Formula (ZC) was synthesized by the method described in Patent Document 3.

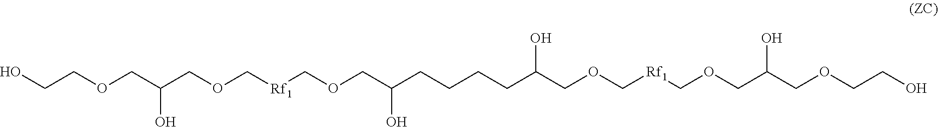

(in Formula (ZC), Rf₁ is the PFPE chain represented by Formula (4-1); and in the two Rf₁'s, h indicating an average degree of polymerization represents 4.5 and i indicating an average degree of polymerization represents 4.5)

### Comparative Example 4

A compound represented by Formula (ZD) was synthesized by the method described in Patent Document 4.

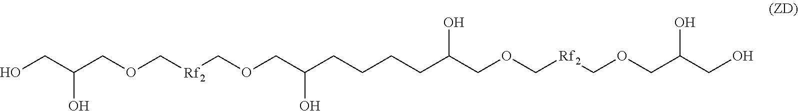

(in Formula (ZD), Rf₂ is the PFPE chain represented by Formula (4-2); and in the two Rf₂'s, j indicating an average degree of polymerization represents 4.5)

### Comparative Example 5

A compound represented by Formula (ZE) was synthesized by the method described in Patent Document 5.

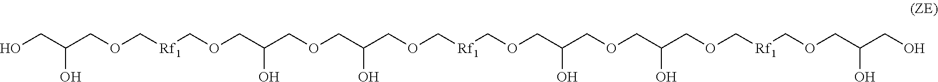

(in Formula (ZE), Rf₁ is the PFPE chain represented by Formula (4-1); and in the three Rf₁'s, h indicating an average degree of polymerization represents 7.0 and i indicating an average degree of polymerization represents 0)

### Comparative Example 6

The compound represented by Formula (ZF) was synthesized by the method described in Patent Document 6.

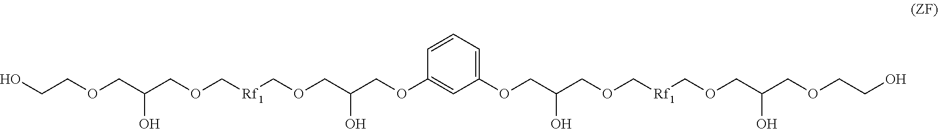

(in Formula (ZF), Rf₁ is the PFPE chain represented by Formula (4-1); and in the two Rf₁'s, h indicating an average degree of polymerization represents 4.5 and i indicating an average degree of polymerization represents 4.5)

### Comparative Example 7

A compound represented by Formula (ZG) was synthesized by the method described in Patent Document 7.

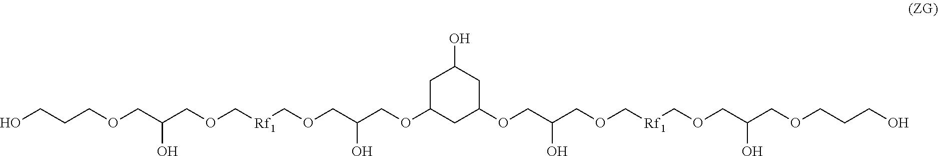

(in Formula (ZG), Rf₁ is the PFPE chain represented by Formula (4-1); and in the two Rf₁'s, h indicating an average degree of polymerization represents 4.5 and i indicating an average degree of polymerization represents 4.5)

The number-average molecular weight (Mn) of the compounds of Examples 1 to 41 and Comparative Examples 1 to 7 obtained as above were measured by the above-described method. The results thereof are shown in Tables 3 and 4.

Next, a lubricating layer forming solution was prepared using the compounds obtained in Examples 1 to 41 and Comparative Examples 1 to 7 by a method shown below. Then, a lubricating layer of a magnetic recording medium was formed by a method shown below using the obtained lubricating layer forming solution, thereby obtaining a magnetic recording medium of each of Examples 1 to 41 and Comparative Examples 1 to 7.

“Lubricating Layer Forming Solution” The compounds obtained in Examples 1 to 41 and Comparative Examples 1 to 7 were each dissolved in a fluorine-based solvent, Vertrel (registered trademark) XF (product name, manufactured by Mitsui DuPont Fluorochemicals Co., Ltd.), and diluted with Vertrel XF such that a film thickness in a case of being applied onto the protective layer was 9.0 Å to 9.5 Å, thereby obtaining a lubricating layer forming solution.

“Magnetic Recording Medium” A magnetic recording medium in which an adhesion layer, a soft magnetic layer, a first underlayer, a second underlayer, a magnetic layer, and a protective layer had been sequentially provided on a substrate having a diameter of 65 mm was prepared. The protective layer was made of carbon.

The lubricating layer forming solutions of Examples 1 to 41 and Comparative Examples 1 to 7 were each applied on the protective layer of the magnetic recording medium, on which each of the layers up to the protective layer had been formed, by a dipping method. Furthermore, the dipping method was performed under the conditions of an immersion speed of 10 mm/sec, an immersion time of 30 see, and a pulling-up speed of 1.2 mm/sec.

Thereafter, the magnetic recording medium on which the lubricating layer forming solution had been applied was placed in a thermostatic chamber and subjected to a thermal treatment at 120° C. for 10 minutes to remove the solvent in the lubricating layer forming solution and to improve the adhesion between the protective layer and the lubricating layer, thereby forming a lubricating layer on the protective layer to obtain a magnetic recording medium.

(Measurement of Film Thickness) The film thickness of the lubricating layer contained in the magnetic recording medium of Examples 1 to 41 and Comparative Examples 1 to 7 obtained as above was measured using a Fourier transform infrared spectrophotometer (FT-IR, product name: Nicolet iS50, manufactured by Thermo Fisher Scientific). The results thereof are shown in Tables 3 and 4.

Next, the magnetic recording media of Examples 1 to 41 and Comparative Examples 1 to 7 were subjected to a chemical substance resistance test and a spin-off characteristic test, each shown below.

### [Chemical Substance Resistance Test]

The degree of contamination of magnetic recording media by environmental substances that generate contaminants in a high-temperature environment was evaluated using a method shown below. An Si ion was used as the environmental substance. The degree of contamination of the magnetic recording medium with the contaminant generated by the environmental substance was evaluated by measuring the amount of Si adsorbed as the amount of the contaminant.

Specifically, the magnetic recording medium to be evaluated was held in a high-temperature environment of a temperature of 85° C. and a humidity of 0% for 240 hours in the presence of a siloxane-based Si rubber. Next, the amount of Si adsorbed to the surface of the magnetic recording medium was analyzed and measured by secondary ion mass spectrometry (SIMS), and the degree of contamination by Si ions was evaluated as the amount of Si adsorbed. The evaluation of the degree of contamination was evaluated based on the following evaluation criteria, using numerical values in a case where the result of the amount of Si adsorbed of Comparative Example 5 was set to 1.00. The results thereof are shown in Tables 3 and 4.

### “Evaluation Criteria of Chemical Substance Resistance”

A+: The amount of Si adsorbed was less than 0.60.
A: The amount of Si adsorbed was 0.60 or more and less than 0.70.
B: The amount of Si adsorbed was 0.70 or more and less than 0.90.
C: The amount of Si adsorbed was 0.90 or more and less than 1.10.
D: The amount of Si adsorbed was 1.10 or more.

### [Spin—Off Characteristic Test]

The magnetic recording medium was mounted on a spin stand and rotated for 72 hours at a rotation speed of 10,000 rpm in an environment of 80° C. Before and after this operation, a film thickness of the lubricating layer at a position of 20 mm from the center of the magnetic recording medium was measured using FT-TR, and a film thickness reduction rate of the lubricating layer before and after the test was calculated. The spin-off characteristics were evaluated in accordance with an evaluation criteria shown below, using the calculated film thickness reduction rate.

### “Evaluation Criteria for Spin—Off Characteristics”

A+: The film thickness reduction rate is less than 2%.
A: The film thickness reduction rate is 2% or more and less than 3%.
B: The film thickness reduction rate is 3% or more and less than 5%.
C: The film thickness reduction rate is 5% or more and less than 10%.
D: The film thickness reduction rate is 10% or more.

### [Comprehensive Evaluations]

From the results of the chemical substance resistance test and the spin-off characteristic test, a comprehensive evaluation was performed based on the following criteria.

### [Comprehensive Evaluations]

A: Both the evaluation of the chemical substance resistance test and the evaluation of the spin-off characteristic test were A+ or A.
B: One of the evaluation of the chemical substance resistance test and the evaluation of the spin-off characteristic test was B, and the other was A+, A, or B.
C: One of the evaluation of the chemical substance resistance test and the evaluation of the spin-off characteristic test was C, and the other was A+, A, B, or C.
D: At least one of the evaluation of the chemical substance resistance test and the evaluation of the spin-off characteristic test was D.

TABLE 3

| | | | Film | Chemical | Spin-off | |
|---|---|---|---|---|---|---|
| | | Molecular | thickness | substance | characteristic | Comprehensive |
| | Compound | weight | (Å) | resistance test | test | evaluation |
| Example 1 | (AA) | 2,416 | 9.2 | A+ | A | A |
| Example 2 | (AB) | 2,388 | 9.4 | A | A | A |
| Example 3 | (AC) | 2,426 | 9.3 | A+ | A | A |
| Example 4 | (AD) | 2,455 | 9.1 | A+ | B | B |
| Example 5 | (AE) | 2,471 | 9.1 | A+ | B | B |
| Example 6 | (AF) | 2,432 | 9.0 | A+ | A | A |
| Example 7 | (AG) | 2,423 | 9.4 | A+ | A | A |
| Example 8 | (AH) | 2,481 | 9.2 | A | A+ | A |
| Example 9 | (AI) | 2,450 | 9.3 | A | A+ | A |
| Example 10 | (AJ) | 2,534 | 9.1 | B | A+ | B |
| Example 11 | (BA) | 2,408 | 9.2 | A+ | A | A |
| Example 12 | (BB) | 2,421 | 9.3 | A+ | A | A |
| Example 13 | (BC) | 2,440 | 9.5 | A+ | A | A |
| Example 14 | (BD) | 2,326 | 9.2 | A+ | A | A |
| Example 15 | (BE) | 2,443 | 9.0 | A+ | A | A |
| Example 16 | (BF) | 2,545 | 9.2 | A | A+ | A |
| Example 17 | (BG) | 2,457 | 9.3 | A | A+ | A |
| Example 18 | (BH) | 2,538 | 9.1 | A | A+ | A |
| Example 19 | (BI) | 2,481 | 9.4 | A | A+ | A |
| Example 20 | (BJ) | 2,436 | 9.2 | A | B | B |
| Example 21 | (BK) | 2,561 | 9.1 | A | B | B |
| Example 22 | (BL) | 2,463 | 9.3 | A | B | B |
| Example 23 | (BM) | 2,334 | 9.1 | A | A | A |
| Example 24 | (BN) | 2,445 | 9.2 | A | A | A |
| Example 25 | (BO) | 2,522 | 9.2 | A | B | B |
| Example 26 | (BP) | 2,558 | 9.1 | A | B | B |
| Example 27 | (BQ) | 2,497 | 9.1 | A+ | A | A |
| Example 28 | (BR) | 2,438 | 9.3 | A+ | A | A |

TABLE 4

| | | | Film | Chemical | | |
|---|---|---|---|---|---|---|
| | | Molecular | thickness | substance | Spin-off | Comprehensive |
| | Compound | weight | (Å) | resistance test | characteristic test | evaluation |
| Example 29 | (CA) | 3,580 | 9.4 | A+ | A | A |
| Example 30 | (CB) | 3,545 | 9.1 | A | A | A |
| Example 31 | (CC) | 3,762 | 9.0 | A+ | B | B |
| Example 32 | (CD) | 3,537 | 9.2 | A+ | A | A |
| Example 33 | (CE) | 3,511 | 9.1 | A+ | A | A |
| Example 34 | (CF) | 3,633 | 9.1 | A | A+ | A |
| Example 35 | (DA) | 3,575 | 9.4 | A+ | A | A |
| Example 36 | (DB) | 3,721 | 9.3 | A | A+ | A |
| Example 37 | (DC) | 3,534 | 9.2 | A+ | A | A |
| Example 38 | (DD) | 3,602 | 9.5 | A+ | A | A |
| Example 39 | (DE) | 3,669 | 9.4 | A+ | A | A |
| Example 40 | (DF) | 3,602 | 9.1 | A+ | A | A |
| Example 41 | (DG) | 3,490 | 9.1 | B | B | B |
| Comparative | (ZA) | 2,206 | 9.2 | D | D | D |
| Example 1 | | | | | | |
| Comparative | (ZB) | 2,278 | 9.4 | D | D | D |
| Example 2 | | | | | | |
| Comparative | (ZC) | 2,322 | 9.1 | C | D | D |
| Example 3 | | | | | | |
| Comparative | (ZD) | 2,292 | 9.4 | C | D | D |
| Example 4 | | | | | | |
| Comparative | (ZE) | 3,413 | 9.0 | C | C | C |
| Example 5 | | | | | | |
| Comparative | (ZF) | 2,450 | 9.2 | D | C | D |
| Example 6 | | | | | | |
| Comparative | (ZG) | 2,474 | 9.1 | C | C | C |
| Example 7 | | | | | | |

As shown in Tables 3 and 4, in the magnetic recording media of Examples 1 to 41, in which R³ is the divalent linking group represented by Formula (2-1) or (2-2) arranged between the perfluoropolyether chains and the fluorine-containing ether compounds (AA) to (AJ), (BA) to (BR), (CA) to (CF), and (DA) to (DG) satisfying Formula (1) were used, the evaluations in all the evaluation items of the chemical substance resistance test and the spin-off characteristic test was any of A+, A, and B. Thus, it was confirmed that the lubricating layers of the magnetic recording media of Examples 1 to 41 had good chemical substance resistance and a high spin-off suppressing effect.

Examples 1 to 10 and 29 to 34 using the compounds (AA) to (AJ) and (CA) to (CF), in which R¹ and R⁴ are each the terminal group represented by Formula (3-1), where p is 0, q is 0, and r is 1, and R² is the perfluoropolyether chain represented by Formula (4-1), were compared. As a result, in Examples 1, 3 to 7, 29, and 31 to 33 using the compound (AA), the compounds (AC) to (AG), the compound (CA), and the compounds (CC) to (CE), in which the number of carbon atoms included in Y in Formula (2) was 3 or more, among the compounds in which R³ is the linking group represented by (2-1), the evaluation of the chemical substance resistance test was A+, indicating good results.

In Examples 8 to 10 and 34 using the compounds (AH) to (AJ) and the compound (CF), in which R³ was the linking group represented by Formula (2-2), the evaluation of the spin-off characteristic test was A+, indicating good results.

On the other hand, in Comparative Examples 1 to 7 using the compounds (ZA) to (ZG), the evaluation in all the evaluation items of the chemical substance resistance test and the spin-off characteristic test was C or D, and the comprehensive evaluation was all C or D.

More specifically, in the compound (ZA) used in Comparative Example 1 and the compound (ZB) used in Comparative Example 2, only one hydroxy group was included in the linking group portion between the perfluoropolyether chains. Therefore, it is considered that since a sufficient interaction between the linking group portion and the protective layer was not obtained, the central portion of the fluorine-containing ether compound was floated up, and thus, contaminants were likely to be entrapped, resulting in D in the chemical substance resistance test. In addition, in the compounds (ZA) and (ZB), bulky perfluoropolyether chains are arranged on both sides of the glycerin structure. Therefore, it is considered that since free motion of the hydroxy group of the glycerin structure is hindered and the hydroxy group is not likely to be involved in the interaction with the active points on the protective layer or the polar group in the other fluorine-containing ether compound present in the lubricating layer, the evaluation in the spin-off characteristic test was D.

In addition, the compound (ZF) used in Comparative Example 6 and the compound (ZG) used in Comparative Example 7 have two or three hydroxy groups in the linking group between the perfluoropolyether chains. However, in the compounds (ZF) and (ZG), since two glycerin structures are bonded to each other through a unit including a rigid ring structure, the motion of the hydroxy group is restricted by the rigid ring structure. Therefore, it is considered that since a sufficient interaction between the linking group portion and the protective layer was not obtained, the central portion of the fluorine-containing ether compound was floated up, and thus, contaminants were likely to be entrapped, the result of the chemical substance resistance test was C or D.

In the compound (ZC) used in Comparative Example 3 and the compound (ZD) used in Comparative Example 4, two hydroxy groups included in the linking group are bonded only through a rigid alkylene chain. Therefore, it is considered that free motion of the hydroxy group included in the linking group is hindered and the hydroxy group is not likely to be involved in the interaction with the polar group in the other fluorine-containing ether compound present in active points on the protective layer or the lubricating layer, and the evaluation in the spin-off characteristic test was D. In addition, in the compound (ZE) used in Comparative Example 5, the perfluoropolyether chains are bonded to each other by a linking group having two hydroxy groups. However, since the distance between the two hydroxy groups contained in the linking group is short, the two hydroxy groups contained in the linking group easily interact with each other in the molecule. Therefore, it is considered that since the two hydroxy groups contained in the linking group are not likely to be involved in the interaction with the polar group included in the other fluorine-containing ether compound molecules present in the lubricating layer, the evaluations in the chemical substance resistance test and the spin-off characteristic test were C.

### INDUSTRIAL APPLICABILITY

By using the lubricant for a magnetic recording medium including the fluorine-containing ether compound of the present invention, a lubricating layer having a good chemical substance resistance and a high spin-off suppressing effect can be formed even in a case where the thickness is small.

### REFERENCE SIGNS LIST

10: magnetic recording medium
11: substrate
12: adhesion layer
13: soft magnetic layer
14: first underlayer
15: second underlayer
16: magnetic layer
17: protective layer
18: lubricating layer

1. A fluorine-containing ether compound represented by Formula (1),
R¹—CH₂—R²[—CH₂—R³—CH₂—R²]ₓ—CH₂—R⁴ (1)
(in Formula (1), x represents an integer of 1 or 2; R² is a perfluoropolyether chain; (x+1) pieces of R²'s may be partially or entirely the same as or different from each other; R³ is a divalent linking group represented by Formula (2); in a case where x is 2, two R³'s may be the same as or different from each other; R¹ and R⁴ are each a terminal group having 1 to 50 carbon atoms, which has 1 to 4 polar groups; and R¹ and R⁴ may be the same as or different from each other) 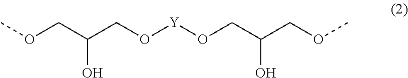
(in Formula (2), Y represents an acyclic divalent saturated hydrocarbon group having 2 to 8 carbon atoms, which may have an ether oxygen atom between carbon atoms).

2. The fluorine-containing ether compound according to claim 1,
wherein Formula (2) is a linking group represented by Formula (2-1) or (2-2), 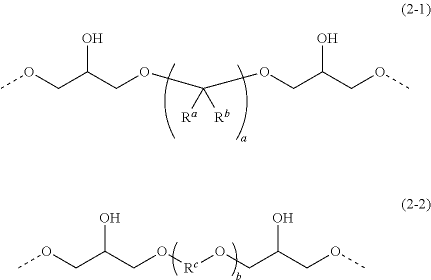
(in Formula (2-1), a represents an integer of 2 to 8; a pieces of R^{a}'s and R^{b}'s each independently represent a hydrogen atom or a methyl group; a total number of carbon atoms included in a pieces of (—CR^{a}R^{b}—)'s is 2 to 8; an oxygen atom at a left terminal of Formula (2-1) is bonded to a methylene group on an R¹ side in Formula (1), and an oxygen atom at a right terminal is bonded to a methylene group on an R⁴ side in Formula (1))
(in Formula (2-2), b represents an integer of 2 to 4; b pieces of R's each independently represent —CH₂CH₂—, —CH₂CH₂CH₂—, —CH(CH₃)CH₂—, or —CH₂CH(CH₃)—; a total number of carbon atoms included in b pieces of R's is 4 to 8; an oxygen atom at a left terminal of Formula (2-2) is bonded to the methylene group on the R¹ side in Formula (1), and an oxygen atom at a right terminal is bonded to the methylene group on the R⁴ side in Formula (1)).

3. The fluorine-containing ether compound according to claim 1,
wherein R¹ and R⁴ in Formula (1) are each independently a terminal group represented by Formula (3), 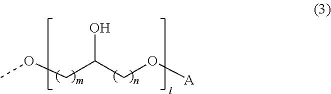
(in Formula (3), 1 represents an integer of 1 to 3; 1 pieces of m's each independently represent an integer of 1 to 6; 1 pieces of n's each independently represent an integer of 1 to 6; at least one of m and n is 1 in one repeating unit; and A represents an alkyl group which may have a polar group, an organic group including a carbon-carbon unsaturated bond, which may have a polar group, or a hydrogen atom).

4. The fluorine-containing ether compound according to claim 1,
wherein R¹ and R⁴ in Formula (1) are each independently a terminal group represented by Formula (3-1) or (3-2), 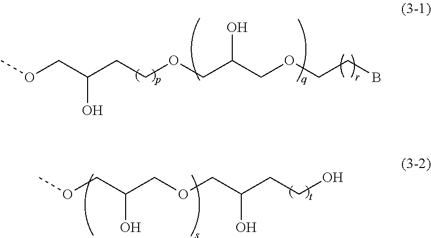
(in Formula (3-1), p represents an integer of 0 to 3, q represents an integer of 0 to 2, and r represents an integer of 1 to 5; a total value of p and r is 1 to 5; and B represents a polar group)
(in Formula (3-2), s represents an integer of 0 to 2, and t represents an integer of 1 to 5).

5. The fluorine-containing ether compound according to claim 1,
wherein R¹ and R⁴ in Formula (1) are the same as each other.

6. The fluorine-containing ether compound according to claim 1,
wherein a total number of the polar groups contained in R¹ and the polar groups contained in R⁴ in Formula (1) is 2 to 6.

7. The fluorine-containing ether compound according to claim 12,
wherein all of the polar groups contained in Rand the polar groups contained in R⁴ in Formula (1) are hydroxy groups.

8. The fluorine-containing ether compound according to claim 1,
wherein (x+1) pieces of R²'s in Formula (1) are each independently a perfluoropolyether chain represented by Formula (4),
—(CF₂)_{w1}—O—(CF₂O)_{w2}—(CF₂CF₂O)_{w3}—(CF₂CF₂CF₂O)_{w4}—(CF₂CF₂CF₂CF₂O)_{w5}—(CF₂)_{w6}— (4)
(in Formula (4), w2, w3, w4, and w5 each indicate an average degree of polymerization and each independently represent 0 to 20, provided that all of w2, w3, w4, and w5 are not 0 at the same time; w1 and w6 each indicate an average value of the number of CF₂'s and each independently represent 1 to 3; and (CF₂O), (CF₂CF₂O), (CF₂CF₂CF₂O), and (CF₂CF₂CF₂CF₂O), which are repeating units in Formula (4) are not particularly limited in the sequence order).

9. The fluorine-containing ether compound according to claim 1,
wherein (x+1) pieces of R²'s in Formula (1) are each independently any one selected from perfluoropolyether chains represented by Formulae (4-1) to (4-4),
—CF₂—(OCF₂CF₂)ₕ—(OCF₂)ᵢ—OCF₂—(4-1)
(in Formula (4-1), h and i each indicate an average degree of polymerization, h represents 1 to 20, and i represents 0 to 20)
—CF₂CF₂—(OCF₂CF₂CF₂)ⱼ—OCF₂CF₂— (4-2)
(in Formula (4-2), j indicates an average degree of polymerization and represents 1 to 15)
—CF₂CF₂CF₂—(OCF₂CF₂CF₂CF₂)ₖ—OCF₂CF₂CF₂— (4-3)
(in Formula (4-3), k indicates an average degree of polymerization and represents 1 to 10)
—(CF₂)_{w7}—O—(CF₂CF₂CF₂O)_{w8}—(CF₂CF₂O)_{w9}—(CF₂)_{w10}— (4-4)
(in Formula (4-4), w8 and w9 each indicate an average degree of polymerization and each independently represent 1 to 20; and w7 and w10 are each an average value indicating the number of CF₂'s and each independently represent 1 or 2).

10. The fluorine-containing ether compound according to claim 1,
wherein the fluorine-containing ether compound has a number-average molecular weight in a range of 500 to 10,000.

11. A lubricant for a magnetic recording medium, comprising:
the fluorine-containing ether compound according to claim 1.

12. A magnetic recording medium comprising, in the following order, on a substrate, at least:
a magnetic layer;
a protective layer; and
a lubricating layer,
wherein the lubricating layer includes the fluorine-containing ether compound according to claim 1.

13. The magnetic recording medium according to claim 12,
wherein an average film thickness of the lubricating layer is 0.5 nm to 2.0 nm.
